SEQUENCE LISTING (28 sequences)

SEQ ID NO: 1 - DNA, 1218 bp, Artificial Sequence
  Arrestin 3A mutant
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctgaggacccagctaaggaaagttatcaggatgcaaatgcagctgctgaggagtttgctcgccataatctgaaagatgcaggagaagctgaggaggggaagagagacaagaatgacgttgatgagtaa

SEQ ID NO: 2 - DNA, 1218 bp, Artificial Sequence
  Arrestin KEQ3A mutant
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtccagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttacaagatcaaggtgaagctcacagtgtcaggctttctgggagagctcacctccagtcacgtcgccactgaggtcccattccgcctcatgcaccctcagcctgaggacccagctaaggaaagttatcaggatgcaaatgcagctgctgaggagtttgctcgccataatctgaaagatgcaggagaagctgaggaggggaagagagacaagaatgacgttgatgagtaa

SEQ ID NO: 3 - DNA, 1692 bp, Homo sapiens
atggatttcgggtctttggagaccgtggtggccaactctgccttcatcgccgcccgaggcagctttgacggcagcagctcccaaccctcccgggacaagaagtacctggccaagctcaagctgcccccgctgtccaagtgtgagtccctccgcgacagcctcagcctggagtttgagagtgtgtgcttggagcagcccatcggcaagaagctctttcagcagttcctacaatcggcagagaagcacctgccggccctggagctctggaaagacatcgaggactatgacacggcagacaatgacctccagccacagaaggcccagaccatcctggcccagtacctggacccccaggccaaactcttctgcagcttcctggatgaggggatagtggcgaagtttaaggaggggcctgtggagatccaggacgggctcttccagcccctgctgcaggccaccctggcacacctgggccaagcccccttccaggagtacctgggcagcctgtacttcctgaggttcctgcagtggaagtggctggaagcccagcccatgggggaggactggttcctggacttcagggtcctggggaaagggggcttcggggaggtgtcggcctgccagatgaaggcgaccggcaagctgtatgcctgcaagaagctgaacaagaagcggctgaagaagaggaagggctaccagggtgctatggtggagaagaagattctgatgaaagtacacagcaggttcatcgtgtctctggcctatgcgtttgaaaccaaagccgacctctgtctggtgatgaccatcatgaacggaggtgacatcaggtaccacatctacaacgtgaatgaggagaaccctggcttcccggagccgcgcgccctcttctacacggcgcagatcatctgcggcctggagcacctgcaccagaggcggatcgtctaccgcgacctcaagcccgagaacgtgctgctggacaatgacggcaatgtccggatctctgaccttgggctggccgtggagctgctggacggacagagcaagaccaagggctacgcagggaccccaggtttcatggcccccgagctcctgcagggcgaggagtacgacttctccgtggactactttgccctgggggtcaccctgtatgagatgattgcggccagaggacccttccgagcccgtggagagaaggtggagaacaaggagctgaagcaccggatcatctcagagcccgtgaagtaccctgataagttcagccaggccagcaaggacttctgcgaggcgctgctggagaaggacccggagaagcgcctggggttcagagatgagacctgcgacaagctccgtgcccaccccctcttcaaggaccttaactggaggcagctggaggctgggatgctgatgccccctttcatcccagactccaaaactgtctacgcaaaggatattcaggacgtgggtgccttttccaccgtcaaaggtgtggcctttgacaaaacagacacagaattctttcaggaatttgccactggcaactgccccatcccctggcaggaggagatgatcgagacgggcatctttggcgagctgaacgtgtggcgctcggacggtcagatgccggacgacatgaagggcatctccgggggctccagctcctcgtccaagtcagggatgtgtctggtttcctaa

SEQ ID NO: 4 - DNA, 1047 bp, Homo sapiens
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtcctaggcttccccataaatttcctcacgctctacgtcaccgtccagcacaagaagctgcgcacgcctctcaactacatcctgctcaacctagccgtggcagatctcttcatggttctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacgtgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctcatatgctgggtgccctacgccagcgtggcattctacatcttcacccaccagggctccaacttcggtcccatcttcatgaccatcccagcgttctttgccaagagcgctgccatctacaaccctgtcatctatatcatgatgaacaagcagttccggaactgtatgctgaccaccatctgctgcggcaagaaccctctgggagatgatgaagccgctgccgccgtggctaaagctgaagctgctcaagtggcccctgcctaa

SEQ ID NO: 5 - DNA, 1047 bp, Homo sapiens
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtcctaggcttccccataaatttcctcacgctctacgtcaccgtccagcacaagaagctgcgcacgcctctcaactacatcctgctcaacctagccgtggcagatctcttcatggttctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacgtgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctcatatgctgggtgccctacgccagcgtggcattctacatcttcacccaccagggctccaacttcggtcccatcttcatgaccatcccagcgttctttgccaagagcgctgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 6 - DNA, 1218 bp, Homo sapiens
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctgaggacccagctaaggaaagttatcaggatgcaaatttagtttttgaggagtttgctcgccataatctgaaagatgcaggagaagctgaggaggggaagagagacaagaatgacgttgatgagtaa

SEQ ID NO: 7 - DNA, 42 bp, Artificial Sequence
  Forward primer of Arr1 3A
gttatcaggatgcaaatgcagctgctgaggagtttgctcgcc

SEQ ID NO: 8 - DNA, 42 bp, Artificial Sequence
  Reverse primer of Arr1 3A
ggcgagcaaactcctcagcagctgcatttgcatcctgataac

SEQ ID NO: 9 - DNA, 35 bp, Artificial Sequence
  Forward primer of K261Q
cgagtgattattacgtccagcccgtggctatggag

SEQ ID NO: 10 - DNA, 35 bp, Artificial Sequence
  Reverse primer of K261Q
ctccatagccacgggctggacgtaataatcactcg

SEQ ID NO: 11 - DNA, 37 bp, Artificial Sequence
  Forward primer of Q332K
gaatcctggtgtcttacaagatcaaggtgaagctcac

SEQ ID NO: 12 - DNA, 38 bp, Artificial Sequence
  Reverse primer of Q332K
gtgagcttcaccttgatcttgtaagacaccaggattcc

SEQ ID NO: 13 - DNA, 36 bp, Artificial Sequence
  Forward primer of E350H
gagagctcacctccagtcacgtcgccactgaggtcc

SEQ ID NO: 14 - DNA, 36 bp, Artificial Sequence
  Reverse primer of E350H
ggacctcagtggcgacgtgactggaggtgagctctc

SEQ ID NO: 15 - DNA, 43 bp, Artificial Sequence
  Forward primer of Arr
ccaggtggccccggctacgcgtgcagccagcgggaagaccagc

SEQ ID NO: 16 - DNA, 53 bp, Artificial Sequence
  Reverse primer of Arr
caggaattcgatatcaagcaccggtttactcatcaacgtcattcttgtctctc

SEQ ID NO: 17 - protein, 5 aa, Artificial Sequence
  F1
GGGGS

SEQ ID NO: 18 - protein, 10 aa, Artificial Sequence
  F2
GGGGSGGGGS

SEQ ID NO: 19 - protein, 15 aa, Artificial Sequence
  F3
GGGGSGGGGSGGGGS

SEQ ID NO: 20 - protein, 7 aa, Artificial Sequence
  R1
AEAAAKA

SEQ ID NO: 21 - protein, 12 aa, Artificial Sequence
  R2
AEAAAKEAAAKA

SEQ ID NO: 22 - protein, 17 aa, Artificial Sequence
  R3
AEAAAKEAAAKEAAAKA

SEQ ID NO: 23 - protein, 17 aa, Artificial Sequence
  SF-1
GGGGSAEAAAKAGGGGS

SEQ ID NO: 24 - protein, 22 aa, Artificial Sequence
  SF-2
GGGGSAEAAAKEAAAKAGGGGS

SEQ ID NO: 25 - protein, 27 aa, Artificial Sequence
  SF-3
GGGGSAEAAAKEAAAKEAAAKAGGGGS

SEQ ID NO: 26 - protein, 32 aa, Artificial Sequence
  SF-4
GGGGSAEAAAKEAAAKEAAAKEAAAKAGGGGS

SEQ ID NO: 27 - protein, 37 aa, Artificial Sequence
  SF-5
GGGGSAEAAAKEAAAKEAAAKEAAAKEAAAKAGGGGS

SEQ ID NO: 28 - protein, 12 aa, Artificial Sequence
  (GSG)4
GSGGSGGSGGSG

(19) United States
(12) Patent Application Publication
(10) Pub. No.: US 2023/0165938 A1
(43) Pub. Date: Jun. 1, 2023

(54) MODULATING OPSIN SIGNALING LIFETIME FOR OPTOGENETIC APPLICATIONS

(71) Applicant: The University of Manchester, Manchester (GB)

(72) Inventors: Robert Lucas, Manchester, Greater Manchester (GB); Jessica Rodgers, Manchester, Greater Manchester (GB); Edward Raymond Ballister, Manchester, Greater Manchester (GB)

(21) Appl. No.: 17/754,721

(22) PCT Filed: Oct. 13, 2020
(86) PCT No.: PCT/GB2020/052551

§ 371 Date: Apr. 8, 2022

(30) Foreign Application Priority Data
Oct. 14, 2019 (GB) 1914826.1

(51) Int. Cl. A61K 38/17; A61K 48/00; A61P 27/02

(52) U.S. Cl. CPC A61K 38/1774; A61K 38/177; A61K 48/0008; A61K 48/0075; A61P 27/02

(57) ABSTRACT

The present disclosure provides compositions comprising an opsin polypeptide and an arrestin polypeptide and their use thereof. Exemplary embodiments provide a composition including an opsin polypeptide, or an opsin polypeptide and an arrestin polypeptide, wherein at least one of the opsin or arrestin polypeptide comprises at least one mutation that increases a temporal resolution of the opsin polypeptide’s response to light. The opsin polypeptide and the arrestin polypeptide can be operably linked or separate. Additionally, use of said compositions for restoring retinal photosensitivity or treating a retinal degenerative condition is also provided.

### INTRODUCTION TO THE INVENTION

The present invention relates to a composition comprising one or more vectors comprising one or more nucleic acid sequences encoding opsin and mutant arrestin; mutant opsin and arrestin; a fusion protein of opsin and arrestin or mutants of one or both of opsin and arrestin; or a meta II decay mutant of opsin. The invention also relates to a composition comprising opsin and mutant arrestin; mutant opsin and arrestin; a fusion protein of opsin and arrestin or mutants of one or both of opsin and arrestin; or a meta II decay mutant of opsin. Also provided is a method of increasing deactivation of an opsin, increasing temporal resolution of an opsin, providing photoreceptor function to a cell, and/or making a cell photoactivatable, comprising expressing in a cell a vector as described herein. Also provided are recombinant cells comprising one or more vectors as disclosed herein, and kits for performing the invention.

### BACKGROUND

Optogenetics is the process of controlling the activity of cells using light by ectopic expression of light sensitive proteins. One class of photopigment for optogenetic control is the retinaldehyde-binding opsins. There are two main classes of opsins: type I microbial opsins that comprise light sensitive ion channels and type II animal opsins that comprise G protein coupled receptors (GPCR). Microbial opsins have short-lived light responses allowing optogenetic control with high temporal resolution. By contrast, animal opsins drive long-lasting light responses when expressed outside of their natural environment, placing a limit on spatiotemporal resolution of optogenetic control. Photoactivation of rhodopsin converts it to the physiologically active Meta II (R*) state, which triggers the rod light response. Meta II is rapidly inactivated by the phosphorylation of C-terminal residues by G-protein receptor kinase (Grk1) and subsequent binding of arrestin. These pigments provide high acuity vision in species across the animal kingdom, but there is a need for improving the temporal resolution of optogenetic control achieved using animal opsins.

### SUMMARY OF THE INVENTION

### Mutant Form of Arrestin in Combination With Opsin

Disclosed herein is a composition comprising a first vector having a first nucleic acid encoding an opsin polypeptide and a second vector having a second nucleic acid encoding a mutant arrestin polypeptide. Disclosed herein is also a composition comprising a vector comprising a first nucleic acid encoding an opsin polypeptide and a second nucleic acid encoding a mutant arrestin polypeptide.

A nucleic acid encoding an opsin polypeptide can be a nucleic acid encoding a wild type opsin polypeptide, its equivalent or a homologous or mutant form thereof from any species. A nucleic acid encoding a mutant arrestin polypeptide can be a nucleic acid encoding a mutant arrestin polypeptide, from any species. In some embodiments, a nucleic acid encoding an opsin polypeptide encodes a human opsin polypeptide, and/or a nucleic acid encoding an arrestin mutant is a nucleic acid encoding a human arrestin mutant.

In some embodiments, the composition is suitable for ocular or subretinal administration.

In some embodiments, the vector is a viral vector. In some embodiments, the viral vector is an adenoassociated viral vector or a modified AAV. In an embodiment, a suitable AAV vector is AAV2. Where a first and second vector are provided, they may be the same type of vector or may be different. For example, a first vector may be a viral vector and a second a non-viral vector, or vice versa. A first vector may be AAV and a second vector may be a non-viral vector, or a viral vector other than AAV. The first and second vectors may both be AAV, of the same or different serotypes. In an embodiment, the vectors are AAV2.

In suitable embodiment, a vector may further comprise a nucleic acid sequence encoding a linker, for operably linking the opsin and arrestin polypeptides expressed by the nucleic acid sequences(s). A nucleic acid sequence encoding a linker may be operably linked to a nucleic acid sequence encoding the opsin and/or arrestin. The nucleic acid sequence may encode a linker selected from the group consisting of a flexible linker, a rigid linker, a semi-flexible linker, an ER/K linker, or a combination thereof. In some embodiments, the linker is a flexible glycine-serine linker, a rigid alpha-helix forming linker, a semi-flexible linker having a rigid linker with flexible ends, and an ER/K linker, or a combination thereof. A linker may be naturally occurring or non-naturally occurring. In some embodiments, the nucleic acid sequences encode opsin polypeptide and the arrestin polypeptides which are separate. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

In some embodiments, the nucleic acid encoding an opsin polypeptide is a nucleic acid encoding an opsin polypeptide which is modified to reduce or minimize phosphorylation by G-protein coupled receptor kinase. In some embodiments, the nucleic acid encoding an opsin polypeptide is a nucleic acid encoding an opsin polypeptide having a mutation associated with a C-terminal phosphorylation site. In an embodiment, the nucleic acid encoding an opsin polypeptide is a nucleic acid encoding an opsin polypeptide having a mutation which increases the rate of meta II decay. In some embodiments, the nucleic acid encoding an opsin polypeptide is a nucleic acid encoding an opsin polypeptide having a mutation associated with a C-terminal phosphorylation site and a mutation which increases the rate of meta II decay.

A mutation associated with a C terminal phosphorylation site may be S333A, T336A, S338A, T340A, T342A, or S343A, or any combination thereof. In some embodiments, the nucleic acid encodes an opsin polypeptide comprising a mutation at S333A, T336A, S338A, T340A, T342A and S343A. In some embodiments, the nucleic acid encodes a human opsin polypeptide comprising a mutation at S333A, T336A, S338A, T340A, T342A and S343A (referred to herein as rod opsin 6A). In some embodiments, the mutation increases the rate of meta II decay. In some embodiments, the nucleic acid encodes an opsin polypeptide comprising a mutation which increases the rate of meta II decay selected from L59Q, Y74F, E122Q, A132L, A132S, Y136F, I189P, Y227F, Y306F, or a combination thereof. In some embodiments, the nucleic acid encodes an opsin polypeptide comprising a mutation selected from L59Q, Y74F, E122Q, A132S, Y136F, I189P, Y306F, or a combination thereof. In a suitable embodiment, the nucleic acid encodes an opsin polypeptide comprising a mutation which is E122Q. In some embodiments, the nucleic acid encodes an opsin polypeptide comprising a combination of i) a mutation associated with a C terminal phosphorylation site and ii) a mutation which increases the rate of meta II decay. Suitably, the mutations or combinations of mutations are as described herein.

In some embodiments, the nucleic acid encoding an arrestin polypeptide is a nucleic acid encoding an arrestin polypeptide having a mutation associated with affinity for unphosphorylated opsin. In some embodiments, nucleic acid encoding an arrestin polypeptide is a nucleic acid encoding an arrestin polypeptide having a mutation selected from L337A, V378A, F379A, K261Q, E350H, or Q332K, or a combination thereof. In some embodiments, the nucleic acid encoding an arrestin polypeptide is a nucleic acid encoding an arrestin polypeptide having a mutation selected from L337A, V378A, F379A, and any combination thereof. In some embodiments, nucleic acid encoding an arrestin polypeptide is a nucleic acid encoding an arrestin polypeptide having a mutation selected from L337A, V378A, F379A, K261Q, E350H, Q332K, and any combination thereof. In some embodiments, the nucleic acid encoding an arrestin polypeptide is a nucleic acid encoding an arrestin polypeptide having mutations L337A, V378A, and F379A (referred to as “Arrestin 3A” or “3A”). In some embodiments, nucleic acid encoding an arrestin polypeptide is a nucleic acid encoding an arrestin polypeptide comprising mutations L337A, V378A, F379A, K261Q, E350H, Q332K (referred to as “Arrestin KEQ3A” or “KEQ3A”).

In some embodiments, the nucleic acid encoding an opsin is a nucleic acid encoding an opsin polypeptide having a mutation associated with a C-terminal phosphorylation site and/or a mutation which increases the rate of meta II decay, and the nucleic acid encoding the arrestin polypeptide is a nucleic acid encoding an arrestin polypeptide having a mutation associated with affinity for unphosphorylated opsin. In some embodiments, the nucleic acid encoding the opsin polypeptide is a nucleic acid encoding an opsin polypeptide comprising a mutation selected from a mutation associated with a C terminal phosphorylation site, for example S333A, T336A, S338A, T340A, T342A, S343A, or any combination thereof and/or a mutation which increases the rate of meta II decay, for example selected from L59Q, Y74F, E122Q, A132L, A132S, Y136F, I189P, Y227F, Y306F, or a combination thereof; and the nucleic acid encoding the arrestin polypeptide is a nucleic acid encoding an arrestin polypeptide comprising a mutation selected from L337A, V378A, F379A, K261Q, E350H, Q332K, or a combination thereof. In some embodiments, the combination of mutations encoded by the nucleic acid(s) may comprise a mutation selected from a) and/or b), and/or c), wherein a) comprises S333A, T336A, S338A, T340A, T342A, S343A; b) comprises L59Q, Y74F, E122Q, A132L, A132S, Y136F, I189P, Y227F, Y306F or a combination thereof and c) comprises L337A, V378A, F379A, K261Q, E350H, Q332K or a combination thereof. In a suitable embodiment, the nucleic acid encoding an opsin polypeptide may be a nucleic acid encoding an opsin polypeptide having an E122Q mutation and the nucleic acid encoding the arrestin polypeptide is a nucleic acid encoding wild type arrestin. In a suitable embodiment, the nucleic acid encoding an opsin polypeptide may be a nucleic acid encoding an opsin polypeptide having an E122Q mutation and the nucleic acid encoding the arrestin polypeptide is a nucleic acid encoding an arrestin polypeptide having a mutation at L337A, V378A and F379A (referred herein as 3A). In a suitable embodiment, the nucleic acid encoding an opsin polypeptide may be a nucleic acid encoding an opsin polypeptide having a mutation at E122Q, S333A, T336A, S338A, T340A, T342A, and S343A and the nucleic acid encoding the arrestin polypeptide is a nucleic acid encoding an arrestin polypeptide having a mutation at L337A, V378A, and F379A (3A). In a suitable embodiment the nucleic acid encoding an opsin polypeptide may be a nucleic acid encoding a human opsin polypeptide having a mutation at E122Q, S333A, T336A, S338A, T340A, T342A, and S343A (E122Q rod 6A) and the nucleic acid encoding the arrestin polypeptide is a nucleic acid encoding an arrestin polypeptide having a mutation at L337A, V378A, and F379A (3A).

In suitable embodiments, the nucleic acid encoding an opsin polypeptide may encode a wild type opsin polypeptide or a variant thereof which does not comprise a mutation associated with a C terminal phosphorylation site or a mutation which increases the rate of meta II decay. Such a mutation is described herein.

Disclosed herein is a recombinant cell comprising a first vector having a first nucleic acid encoding an opsin polypeptide and a second vector having a second nucleic acid encoding a mutant arrestin polypeptide. Disclosed herein is also a recombinant cell comprising a vector comprising a first nucleic acid encoding an opsin polypeptide and a second nucleic acid encoding a mutant arrestin polypeptide. Suitably, the vector(s) are as described herein.

Also disclosed herein is a recombinant cell comprising a first nucleic acid encoding an opsin polypeptide and a second nucleic acid encoding a mutant arrestin polypeptide. Suitably, the nucleic acid sequences are as described herein. In a suitable embodiment, the first and/or second nucleic acid may be integrated into the genome of the cell.

Also disclosed herein is a recombinant cell as defined herein, comprising an expressed nucleic acid sequence as defined herein.

In some embodiments, the cell is a neuronal cell, suitably a neuronal stem cell. Suitably, the cell is a retinal cell. In suitable embodiments, the cell is an inner retinal. In suitable embodiments, the cell is an ON-bipolar cell, an OFF-bipolar cell, a horizontal cell, a ganglion cell and/or an amacrine cell. Suitably, the cell is a human cell.

Disclosed herein is a composition comprising an opsin polypeptide and a mutant arrestin polypeptide. The opsin polypeptide can be a wild type opsin polypeptide, its equivalent or a homologous or mutant form thereof from any species. The mutant arrestin polypeptide can be a mutant arrestin polypeptide, from any species. In some embodiments, the opsin polypeptide is a human opsin, and/or the arrestin is a human arrestin.

In some embodiments, the arrestin polypeptide has one or more mutations that help increase its binding affinity to unphosphorylated opsin. In some embodiments, the opsin polypeptide has one or more mutations associated with a C terminal phosphorylation site. In some embodiments, the opsin polypeptide has one or more mutations which increases the rate of meta II decay.

The opsin polypeptide can be operably linked to the arrestin polypeptide through a linker, or the opsin polypeptide can be separate from the arrestin polypeptide. In some embodiments, the linker is a flexible linker, a rigid linker, a semi-flexible linker, an ER/K linker, or a combination thereof. In some embodiments, the linker is a flexible glycine-serine linker, a rigid alpha-helix forming linker, a semi-flexible linker having a rigid linker with flexible ends, and an ER/K linker, or a combination thereof. A linker may be an ER/K semi flexible linker. A linker may be naturally occurring or non-naturally occurring. In some embodiments, the opsin polypeptide and the arrestin polypeptide are separate. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

In some embodiments, the composition is suitable for ocular or subretinal administration.

In some embodiments, the opsin has one or more mutations associated with a C terminal phosphorylation site and/or the opsin polypeptide has one or more mutations which increases the rate of meta II decay, and the arrestin polypeptide has one or more mutations that help increase its binding affinity to unphosphorylated opsin. In some embodiments, the arrestin polypeptide has one or more mutations that help increase its binding affinity to unphosphorylated opsin, and the opsin polypeptide is wild type opsin or a variant thereof which does not comprise a mutation associated with a C terminal phosphorylation site or a mutation which increases the rate of meta II decay.

In some embodiments, the opsin is modified to reduce or minimize phosphorylation by G-protein coupled receptor kinase. In some embodiments, the opsin comprises a mutation associated with a C-terminal phosphorylation site. A mutation associated with a C terminal phosphorylation site may be S333A, T336A, S338A, T340A, T342A, S343A, or any combination thereof. In some embodiments, the opsin comprises a mutation at S333A, T336A, S338A, T340A, T342A and S343A. In some embodiments, the opsin is human rod opsin and comprises a mutation at S333A, T336A, S338A, T340A, T342A and S343A (referred to herein as rod opsin 6A). In some embodiments, the opsin comprises a mutation which increases the rate of meta II decay. A mutation which increases the rate of meta II decay may be selected from L59Q, Y74F, E122Q, A132L, A132S, Y136F, I189P, Y227F, Y306F, or a combination thereof. In some embodiments, the mutation is selected from L59Q, Y74F, E122Q, A132S, Y136F, I189P, Y306F, or a combination thereof. In a suitable embodiment, an opsin mutation is E122Q. In some embodiments, the opsin comprises a combination of i) a mutation associated with a C terminal phosphorylation site and ii) a mutation which increases the rate of meta II decay.

In some embodiments, the arrestin comprises a mutation associated with affinity for unphosphorylated opsin. In some embodiments, the arrestin polypeptide comprises a mutation selected from L337A, V378A, F379A, K261Q, E350H, Q332K, or a combination thereof. In some embodiments, the arrestin polypeptide comprises a mutation selected from L337A, V378A, F379A, and any combination thereof. In some embodiments, the arrestin polypeptide comprises a mutation selected from L337A, V378A, F379A, K261Q, E350H, Q332K, and any combination thereof. In some embodiments, the arrestin polypeptide comprises mutations L337A, V378A, and F379A (referred to as 3A). In some embodiments, the arrestin polypeptide comprises the mutations L337A, V378A, F379A, K261Q, E350H, Q332K (referred to as KEQ3A).

In some embodiments, the opsin comprises a mutation associated with a C-terminal phosphorylation site and/or a mutation which increases the rate of meta II decay, and the arrestin comprises a mutation associated with affinity for unphosphorylated opsin. In some embodiments, the opsin comprises a mutation selected from mutant associated with a C terminal phosphorylation site, for example S333A, T336A, S338A, T340A, T342A, S343A, or any combination thereof and/or a mutation which increases the rate of meta II decay, for example selected from L59Q, Y74F, E122Q, A132L, A132S, Y136F, I189P, Y227F, Y306F, or a combination thereof; and the arrestin comprises a mutation selected from L337A, V378A, F379A, K261Q, E350H, Q332K, or a combination thereof. In some embodiments, the combination of mutations may comprise a mutation selected from a) and/or b), and/or c), wherein a) comprises S333A, T336A, S338A, T340A, T342A, S343A; b) comprises L59Q, Y74F, E122Q, A132L, A132S, Y136F, I189P, Y227F, Y306F or a combination thereof and c) comprises L337A, V378A, F379A, K261Q, E350H, Q332K or a combination thereof. In a suitable embodiment, an opsin mutation is E122Q, in combination with wild type arrestin. In a suitable embodiment, an opsin mutation is E122Q, in combination with an arrestin mutation at L337A, V378A and F379A (referred herein as 3A). In a suitable embodiment, the opsin mutation is E122Q, S333A, T336A, S338A, T340A, T342A, and S343A in combination with an arrestin mutation at L337A, V378A, and F379A (3A). In a suitable embodiment the opsin is human rod opsin and the mutation is is E122Q, S333A, T336A, S338A, T340A, T342A, and S343A (E122Q rod 6A) in combination with an arrestin mutation at L337A, V378A, and F379A (3A).

In suitable embodiments, the opsin polypeptide may be a wild type opsin polypeptide or a variant thereof which does not comprise a mutation associated with a C terminal phosphorylation site or a mutation which increases the rate of meta II decay. Such a mutation is described herein.

Disclosed herein, is a kit comprising i) a nucleic acid sequence encoding an opsin polypeptide, ii) a nucleic acid sequence encoding a phosphorylation independent arrestin mutant, and optionally ii) an extracellular matrix degradation enzyme. Suitably, the opsin polypeptide and the arrestin mutant are as described above. In some embodiments, the kit further comprises instructions for use, a dosage regimen, one or more fine needles, one or more syringes, and solvent.

### Mutant Forms of Opsin in Combination With Arrestin

Disclosed herein is a composition comprising a first vector having a first nucleic acid encoding a mutant opsin polypeptide and a second vector having a second nucleic acid encoding an arrestin polypeptide. Disclosed herein is also a composition comprising a vector comprising a first nucleic acid encoding a mutant opsin polypeptide and a second nucleic acid encoding an arrestin polypeptide.

The nucleic acid encoding the opsin polypeptide can be a nucleic acid encoding a mutant opsin polypeptide from any species. The nucleic acid encoding the arrestin polypeptide can be a nucleic acid encoding a wild type arrestin polypeptide, homologue or equivalent from any species. In some embodiments, the nucleic acid encoding the opsin polypeptide encodes a human mutant opsin polypeptide, and/or the nucleic acid encoding the arrestin is a nucleic acid encoding a human arrestin. In a suitable embodiment the nucleic acid encodes a human wild type arrestin polypeptide.

In some embodiments, the vector is a viral vector. In some embodiments, the viral vector is an adenoassociated viral vector or a modified AAV. In an embodiment, a suitable AAV vector is AAV2. Where a first and second vector are provided, they may be the same type of vector or may be different. For example, a first vector may be a viral vector and a second a non-viral vector, or vice versa. A first vector may be AAV and a second vector may be a non-viral vector, or a viral vector other than AAV. The first and second vectors may both be AAV, of the same or different serotypes. In an embodiment, the vectors are AAV2.

In suitable embodiment, a vector may further comprise a nucleic acid sequence encoding a linker, for operably linking the opsin and arrestin polypeptides. A nucleic acid sequence encoding a linker may be operably linked to a nucleic acid sequence encoding the opsin and/or arrestin. The nucleic acid sequence may encode a linker selected from the group consisting of a flexible linker, a rigid linker, a semi-flexible linker ER/K linker, or a combination thereof. In some embodiments, the linker is a flexible glycine-serine linker, a rigid alpha-helix forming linker, a semi-flexible linker having a rigid linker with flexible ends, and a ER/K linker, or a combination thereof. A linker may be an ER/K semi flexible linker. A linker may be naturally occurring or non-naturally occurring. In some embodiments, the opsin polypeptide and the arrestin polypeptide are separate. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

In some embodiments, the composition is suitable for ocular or subretinal administration.

In some embodiments, the nucleic acid encoding an opsin polypeptide is a nucleic acid encoding an opsin polypeptide which is modified to reduce or minimize phosphorylation by G-protein coupled receptor kinase. In some embodiments, the nucleic acid encoding an opsin polypeptide is a nucleic acid encoding an opsin polypeptide having a mutation associated with a C-terminal phosphorylation site. In an embodiment, the nucleic acid encoding an opsin polypeptide is a nucleic acid encoding an opsin polypeptide having a mutation which increases the rate of meta II decay. In some embodiments, the nucleic acid encoding an opsin polypeptide is a nucleic acid encoding an opsin polypeptide having a mutation associated with a C-terminal phosphorylation site and a mutation which increases the rate of meta II decay.

A mutation associated with a C terminal phosphorylation site may be S333A, T336A, S338A, T340A, T342A, or S343A, or any combination thereof. In some embodiments, the nucleic acid encodes an opsin polypeptide comprising a mutation at S333A, T336A, S338A, T340A, T342A and S343A. In some embodiments, the nucleic acid encodes a human opsin polypeptide comprising a mutation at S333A, T336A, S338A, T340A, T342A and S343A (referred to herein as rod opsin 6A). In some embodiments, the mutation increases the rate of meta II decay. In some embodiments, the nucleic acid encodes an opsin polypeptide comprising a mutation which increases the rate of meta II decay selected from L59Q, Y74F, E122Q, A132L, A132S, Y136F, I189P, Y227F, Y306F, or a combination thereof. In some embodiments, the nucleic acid encodes an opsin polypeptide comprising a mutation selected from L59Q, Y74F, E122Q, A132S, Y136F, I189P, Y306F, or a combination thereof. In a suitable embodiment, the nucleic acid encodes an opsin polypeptide comprising a mutation which is E122Q. In some embodiments, the nucleic acid encodes an opsin polypeptide comprising a combination of i) a mutation associated with a C terminal phosphorylation site and ii) a mutation which increases the rate of meta II decay. Suitably, the mutations or combinations of mutations are as described herein.

In suitable embodiments, the nucleic acid encoding an arrestin polypeptide may encode a wild type arrestin polypeptide or a variant thereof which does not comprise a mutation associated with affinity for unphosphorylated opsin. Such a mutation is described herein.

Disclosed herein is a recombinant cell comprising a first vector having a first nucleic acid encoding a mutant opsin polypeptide and a second vector having a second nucleic acid encoding an arrestin polypeptide. Disclosed herein is also a composition comprising a vector comprising a first nucleic acid encoding a mutant opsin polypeptide and a second nucleic acid encoding an arrestin polypeptide. Suitably, the mutant opsin and wild type arrestin are as described herein.

Also disclosed herein is a recombinant cell comprising a first nucleic acid encoding a mutant opsin polypeptide and a second nucleic acid encoding an arrestin polypeptide. Suitably, the nucleic acid encoding the mutant opsin and wild type arrestin are as described herein. In a suitable embodiment, the first and/or second nucleic acid may be integrated into the genome of the cell.

Also disclosed herein is a recombinant cell as defined herein, comprising an expressed nucleic acid sequence as defined herein.

In some embodiments, the cell is a neuronal cell, suitably a neuronal stem cell. Suitably, the cell is a retinal cell. In suitable embodiments, the cell is an inner retinal. In suitable embodiments, the cell is an ON-bipolar cell, an OFF-bipolar cell, a horizontal cell, a ganglion cell and/or an amacrine cell. Suitably, the cell is a human cell.

Disclosed herein is a composition comprising a mutant opsin polypeptide and an arrestin polypeptide. The mutant opsin polypeptide can be from any species. The arrestin polypeptide can be a wild type arrestin polypeptide, from any species. In some embodiments, the opsin polypeptide is a human opsin, and/or the arrestin is a human arrestin, suitably a human wild type arrestin.

In some embodiments, the opsin polypeptide has one or more mutations associated with a C terminal phosphorylation site. In some embodiments, the opsin polypeptide has one or more mutations which increases the rate of meta II decay. Suitable mutants and suitable combinations of mutations are as described herein.

The opsin polypeptide can be operably linked to the arrestin polypeptide through a linker, or the opsin polypeptide can be separate from the arrestin polypeptide. In some embodiments, the linker is a flexible linker, a rigid linker, a semi-flexible linker ER/K linker, or a combination thereof. In some embodiments, the linker is a flexible glycine-serine linker, a rigid alpha-helix forming linker, a semi-flexible linker having a rigid linker with flexible ends, and a ER/K linker, or a combination thereof. A linker may be an ER/K semi flexible linker. A linker may be naturally occurring or non-naturally occurring. In some embodiments, the opsin polypeptide and the arrestin polypeptide are separate. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

In some embodiments, the composition is suitable for ocular or subretinal administration.

Disclosed herein, is a kit comprising i) a first nucleic acid encoding a mutant opsin polypeptide and a second vector having a second nucleic acid encoding an arrestin polypeptide; or a vector comprising a first nucleic acid encoding a mutant opsin polypeptide and a second nucleic acid encoding an arrestin polypeptide; and optionally ii) an extracellular matrix degradation enzyme. Suitably, the nucleic acids are as described above. In some embodiments, the kit further comprises instructions for use, a dosage regimen, one or more fine needles, one or more syringes, and solvent.

### A Fusion Protein Comprising Opsin and Arrestin

Disclosed herein, in some embodiments, is a photoactivatable chimeric polypeptide comprising: (a) an opsin segment comprising an opsin polypeptide or fragment or mutant thereof, and (b) a modulatory segment capable of altering the photoactivity of the opsin segment.

In some embodiments, the opsin segment comprises a full-length opsin polypeptide. The opsin polypeptide may be the wild type polypeptide or a fragment thereof, or may be a mutant thereof In some embodiments, the opsin segment comprises a rod opsin polypeptide or fragment or mutant thereof. In some embodiments, the opsin segment comprises a rod opsin 6A polypeptide or fragment or modification thereof, wherein the rod opsin 6A comprises a mutant of rod opsin designed to minimize phosphorylation by G protein coupled receptor kinase. In some embodiments, the opsin segment comprises at least one mutation modulating the photoactivity of the photoactivatable chimeric polypeptide. In some embodiments, the opsin segment comprises at least one mutation increasing a rate of Schiff base hydrolysis or a rate of meta-II decay. In some embodiments, the opsin segment comprises a mutation at S333A, T336A, S338A, T340A, T342A and S343A (referred to herein as rod opsin 6A) and a mutation which increases the rate of meta II decay, suitably E122Q. Suitably, the mutants of the opsin segments are as described herein.

In some embodiments, the modulatory segment comprises an arrestin polypeptide or fragment or modification thereof. The arrestin polypeptide may be the wild type polypeptide or a fragment thereof, or may be a mutant thereof. In some embodiments, the modulatory segment comprises an arrestin polypeptide further comprising at least one mutation enabling phosphorylation-independent binding of the opsin segment, suitably as described herein. In some embodiments, the modulatory segment comprises a human rod arrestin polypeptide or fragment or modification thereof. In some embodiments, the opsin segment is fused to the modulatory segment by a polypeptide linker. In a suitable embodiment, the photoactivatable chimeric polypeptide comprises a rod opsin polypeptide or fragment or modification thereof and a human rod arrestin polypeptide or fragment or modification thereof, suitably wherein the opsin segment is fused to the modulatory segment by a polypeptide linker.

In some embodiments, the linker is a flexible linker, a rigid linker, a semi-flexible linker, ER/K linker, or a combination thereof. In some embodiments, the linker is a flexible glycine-serine linker, a rigid alpha-helix forming linkers, a semi-flexible linker having a rigid linker with flexible ends, and a ER/K linker, or a combination thereof. A linker may be an ER/K semi flexible linker. A linker may be naturally occurring or non-naturally occurring. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

In some embodiments, the composition is suitable for ocular or subretinal administration.

Disclosed herein is a composition comprising a vector comprising a nucleic acid encoding an opsin polypeptide and an arrestin polypeptide, wherein the opsin polypeptide and the arrestin polypeptide are operably linked by a peptide linker. Suitably, the nucleic acids encoding the opsin polypeptide and arrestin polypeptide are as described herein. The nucleic acid may encode an opsin polypeptide-linker-arrestin polypeptide or arrestin polypeptide-linker-opsin polypeptide, suitably as a single polypeptide sequence. The linker may be a nucleic acid encoding a suitable linker as described herein. Suitably, the vector comprises a nucleic acid sequence encoding a photoactivatable chimeric polypeptide as described above.

Suitably, the opsin polypeptide and/or the arrestin polypeptide may be wild type polypeptides. Suitably, the opsin polypeptide and/or the arrestin polypeptide may be encoded by wild type mucleic acid sequences. Suitably, the nucleic acid encoding the opsin polypeptide may encode a wild type polypeptide and the nucleic acid sequence encoding the arrestin polypeptide may encode a mutant arrestin polypeptide. Suitably, the nucleic acid encoding the arrestin polypeptide may encode a wild type arrestin polypeptide and the nucleic acid sequence encoding the opsin polypeptide may encode a mutant opsin polypeptide.

In some embodiments, the vector is a viral vector. In some embodiments, the viral vector is an adenoassociated viral vector or a modified AAV. In an embodiment, a suitable AAV vector is AAV2. Where a first and second vector are provided, they may be the same type of vector or may be different. For example, a first vector may be a viral vector and a second a non-viral vector, or vice versa. A first vector may be AAV and a second vector may be a non-viral vector, or a viral vector other than AAV. The first and second vectors may both be AAV, of the same or different serotypes. In an embodiment, the vectors are AAV2.

Also provided is a polynucleotide comprising a nucleic acid sequence encoding the photoactivatable chimeric polypeptide. In some embodiments, the polynucleotide is a polynucleotide expression vector comprising a nucleic acid sequence encoding the photoactivatable chimeric polypeptide.

Disclosed herein, in some embodiments, is a composition comprising: (a) a polynucleotide comprising a nucleic acid sequence encoding the photoactivatable chimeric polypeptide. The composition may further comprise a gene delivery vector. In some embodiments, the gene delivery vector is a viral vector, a physical delivery vector, or a chemical delivery vector. In some embodiments the composition further comprises: (c) a polynucleotide comprising a nucleic acid sequence encoding a GRK1 G-protein coupled receptor kinase.

In some embodiments, the composition is suitable for ocular or subretinal administration.

Disclosed herein is a recombinant cell comprising a vector comprising a nucleic acid encoding an opsin polypeptide and an arrestin polypeptide, wherein the opsin polypeptide and the arrestin polypeptide are operably linked by a peptide linker. Suitably, the opsin and arrestin are as described herein.

In a suitable embodiment, the nucleic acid may be integrated into the genome of the cell. Also disclosed herein is a recombinant cell expressing the nucleic acid encoding an opsin polypeptide and an arrestin polypeptide.

Disclosed herein, in some cases, is a cell comprising the photoactivatable chimeric polypeptide. In some cases, the cell further comprises a GRK1 G-protein coupled receptor kinase.

In some embodiments, the cell is a neuronal cell, suitably a neuronal stem cell. Suitably, the cell is a retinal cell. In suitable embodiments, the cell is an inner retinal. In suitable embodiments, the cell is an ON-bipolar cell, an OFF-bipolar cell, a horizontal cell, a ganglion cell and/or an amacrine cell. Suitably, the cell is a human cell.

In some embodiments, the polynucleotide encoding an opsin polypeptide is operably linked to the nucleic acid encoding the mutant arrestin polypeptide.

Disclosed herein, is a kit comprising i) a nucleic acid encoding an opsin polypeptide and an arrestin polypeptide, wherein the opsin polypeptide and the arrestin polypeptide are operably linked by a peptide linker; and optionally ii) an extracellular matrix degradation enzyme. Suitably, the nucleic acids are as described above. Suitably, the nucleic acid encodes the photoactivatable chimeric polypeptide as described above. In some embodiments, the kit further comprises instructions for use, a dosage regimen, one or more fine needles, one or more syringes, and solvent.

Disclosed herein, is a kit comprising a composition comprising (a) a polynucleotide comprising a nucleic acid sequence encoding a photoactivatable chimeric polypeptide as defined herein, and (b) an acceptable gene delivery vector. In some embodiments, the acceptable gene delivery vector is a viral vector, a physical delivery vector, or a chemical delivery vector. In some embodiments the composition further comprises: (c) a polynucleotide comprising a nucleic acid sequence encoding a GRK1 G-protein coupled receptor kinase. In some embodiments, the kit further comprises instructions for use, a dosage regimen, one or more fine needles, one or more syringes, and solvent.

### A Meta II Decay Mutant of Opsin

Disclosed herein is a recombinant cell comprising a vector comprising a nucleic acid encoding an opsin polypeptide comprising a mutation which increases the rate of meta II decay. Suitably, the nucleic acid encoding the opsin polypeptide is as described herein. Suitably, the mutations which increases the rate of meta II decay are as described herein.

The nucleic acid sequence may be provided in a vector, suitably as described herein. In a suitable embodiment, the nucleic acid may be integrated into the genome of the cell.

In some embodiments, the cell is modified to express the nucleic acid. In some embodiments, the cell is a retinal cell. In suitable embodiments, the cell is an inner retinal. In suitable embodiments, the cell is an ON-bipolar cell, an OFF-bipolar cell, a horizontal cell, a ganglion cell and/or an amacrine cell.

Disclosed herein, is a kit comprising i) a vector comprising a nucleic acid encoding an opsin polypeptide comprising a mutation which increases the rate of meta II decay; and optionally ii) an extracellular matrix degradation enzyme. Suitably, the nucleic acids are as described above. Suitably, the nucleic acid encoding the opsin polypeptide is as described herein. Suitably, the mutations which increases the rate of meta II decay are as described herein. In some embodiments, the kit further comprises instructions for use, a dosage regimen, one or more fine needles, one or more syringes, and solvent.

### A Composition Comprising GRK1

Disclosed herein, in some embodiments, is a composition comprising an opsin polypeptide and a G-protein coupled receptor kinase GRK1 polypeptide. In some embodiments, the opsin polypeptide is operably linked to the G-protein coupled receptor kinase GRK1 polypeptide through a linker.

In suitable embodiments, the opsin polypeptide is as described herein. In a suitable embodiment the composition may further comprise an arrestin polypeptide. The arrestin polypeptide may be operably linked to the opsin or GRK1 polypeptide. The arrestin polypeptide may be as described herein.

In some embodiments, the linker is a flexible linker, a rigid linker, a semi-flexible linker, ER/K linker, or a combination thereof. In some embodiments, the linker is a flexible glycine-serine linker, a rigid alpha-helix forming linkers, a semi-flexible linker having a rigid linker with flexible ends, and ER/K linker, or a combination thereof. A linker may be an ER/K semi flexible linker. In some embodiments, the opsin polypeptide and G-protein coupled receptor kinase GRK1 polypeptide are separate. A linker may be naturally occurring or non-naturally occurring. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

Disclosed herein, in some embodiments, is a composition comprising a first vector having a nucleic acid encoding an opsin polypeptide, a second vector having a nucleic acid encoding a G-protein coupled receptor kinase GRK1 polypeptide, and a third vector comprising a nucleic acid encoding an arrestin polypeptide. Disclosed herein, in some embodiments, is a composition comprising a first polynucleotide encoding an opsin polypeptide, a second polynucleotide encoding a G-protein coupled receptor kinase GRK1 polypeptide, and a third nucleic acid encoding an arrestin polypeptide, wherein the two or more of the first, second, and third polynucleotides can be on the same vector or different vectors. Suitably, the polynucleotides encoding the opsin polypeptide, arrestin polypeptide and GRK1 polypeptide may be as described herein.

In some embodiments, the composition is suitable for ocular or subretinal administration.

Disclosed herein is a kit comprising i) a nucleic acid sequence encoding a opsin polypeptide, ii) a nucleic acid sequence encoding a G-protein coupled receptor kinase GRK1, iii) a nucleic acid encoding an arrestin polypeptide, and optionally iv) an extracellular matrix degradation enzyme.

### A Method of Deactivation

Disclosed herein, in some embodiments, is a method of increasing deactivation of an opsin in a cell, comprising contacting the opsin to a phosphorylation independent arrestin mutant.

Also disclosed in some embodiments is a method of increasing deactivation of an opsin, comprising administering to the cell an effective amount of a composition comprising a first vector having a first nucleic acid encoding an opsin polypeptide and a second vector having a second nucleic acid encoding a mutant arrestin polypeptide. Also disclosed in some embodiments is a method of increasing deactivation of an opsin in a cell, comprising administering to the cell an effective amount of a composition comprising a vector comprising a first nucleic acid encoding an opsin polypeptide and a second nucleic acid encoding an arrestin polypeptide.

Also disclosed herein is a method of increasing deactivation of an opsin, comprising administering to a cell a nucleic acid sequence encoding an opsin comprising a mutation which increases the rate of meta II decay. The nucleic acid sequence may encode an opsin having a mutation selected from L59Q, Y74F, E122Q, A132L, A132S, Y136F, I189P, Y227F, Y306F, or a combination thereof. In some embodiments, the nucleic acid sequence may encode an opsin having a mutation selected from L59Q, Y74F, E122Q, A132S, Y136F, I189P, Y306F, or a combination thereof. In a suitable embodiment, the nucleic acid sequence may encode an opsin having an E122Q mutation. Suitably, the nucleic acid sequence may encode an opsin meta II decay mutant opsin meta further comprising a mutation at S333A, T336A, S338A, T340A, T342A and S343A (referred to herein as rod opsin 6A). The nucleic acid sequence may be provided in a vector, suitably as described herein.

Disclosed herein is a method of increasing deactivation of an opsin, comprising administering to a cell an opsin comprising a mutation which increases the rate of meta II decay. Suitable mutations may be selected from L59Q, Y74F, E122Q, A132L, A132S, Y136F, I189P, Y227F, Y306F, or a combination thereof. In some embodiments, the mutation may be selected from L59Q, Y74F, E122Q, A132S, Y136F, I189P, Y306F, or a combination thereof. In a suitable embodiment, the mutation may be E122Q.

Suitably, the method comprises contacting a cell comprising the opsin with a suitable composition of the present invention as described herein.

Suitably, the method of increasing deactivation of an opsin, comprising administering to a cell a nucleic acid sequence encoding an opsin comprising a mutation which increases the rate of meta II decay as described above may further comprise administering to the cell a nucleic acid sequence encoding arrestin. Suitably, the nucleic acid encodes wild type arrestin.

Suitably, there is also provided a method of increasing deactivation of an opsin, comprising administering to a cell an opsin comprising a mutation which increases the rate of meta II decay as described above, and further comprising administering to the cell wild type arrestin.

Suitably, the method comprises contacting a cell comprising the opsin with a suitable composition of the present invention as described herein.

Also disclosed herein is a method of increasing deactivation of an opsin, comprising administering to a cell a nucleic acid sequence encoding a polynucleotide comprising a nucleic acid sequence encoding the photoactivatable chimeric polypeptide as described herein.

Disclosed herein is a method of increasing deactivation of an opsin, comprising contacting the opsin to a G-protein coupled receptor kinase GRK1, or to GRK1 and an arrestin.

Suitably, a cell is as described herein.

### A Method of Increasing Temporal Resolution

Also disclosed in some embodiments is a method of increasing a temporal resolution of an opsin light response, comprising administering to the cell an effective amount of a composition comprising a first vector having a first nucleic acid encoding an opsin polypeptide and a second vector having a second nucleic acid encoding a mutant arrestin polypeptide.

Also disclosed in some embodiments is a method of increasing a temporal resolution of an opsin light response in a cell, comprising administering to the cell an effective amount of a composition comprising a vector comprising a first nucleic acid encoding an opsin polypeptide and a second nucleic acid encoding an arrestin polypeptide.

Suitably, the method comprises contacting a cell comprising the opsin with a suitable composition of the present invention as described herein.

Disclosed herein is a method of increasing a temporal resolution of an opsin light response, comprising administering to a cell an opsin comprising a mutation which increases the rate of meta II decay. Suitable mutations may be selected from L59Q, Y74F, E122Q, A132L, A132S, Y136F, I189P, Y227F, Y306F, or a combination thereof. In some embodiments, the mutation may be selected from L59Q, Y74F, E122Q, A132S, Y136F, I189P, Y306F, or a combination thereof. In a suitable embodiment, the mutation may be E122Q.

Also disclosed herein is a method of increasing a temporal resolution of an opsin light response, comprising administering to a cell a nucleic acid sequence encoding an opsin comprising a mutation which increases the rate of meta II decay. The nucleic acid sequence may encode an opsin having a mutation selected from L59Q, Y74F, E122Q, A132L, A132S, Y136F, I189P, Y227F, Y306F, or a combination thereof. In some embodiments, the nucleic acid sequence may encode an opsin having a mutation selected from L59Q, Y74F, E122Q, A132S, Y136F, I189P, Y306F, or a combination thereof. In a suitable embodiment, the nucleic acid sequence may encode an opsin having an E122Q mutation. The nucleic acid sequence may be provided in a vector, suitably as described herein.

Suitably, the cell is a neuronal cell, suitably a neuronal stem cell. Suitably, the cell is a retinal cell. Suitably, the cell is an inner retinal cell, for example an ON-bipolar cell, an OFF-bipolar cell, a horizontal cell, a ganglion cell and/or an amacrine cell. Suitably, the cell is a human cell.

Suitably, the opsin is native to the cell. Alternatively, the opsin may be heterologous to the cell, meaning the that opsin is not naturally expressed in the cell but has been introduced into the cell for example by recombinant gene technology.

Disclosed herein, in some embodiments, is a method of increasing a temporal resolution of an opsin light response in a cell comprising: (a) delivering to the cell a polynucleotide comprising a nucleic acid sequence encoding the photoactivatable chimeric polypeptide, and (b) expressing in the cell the photoactivatable chimeric polypeptide. In some embodiments, the polynucleotide of (a) comprises delivering a polynucleotide expression vector comprising the nucleic acid sequence encoding the photoactivatable chimeric polypeptide. In some embodiments, the method (a) further comprises delivering a polynucleotide comprising a nucleic acid sequence encoding a GRK1 G-protein coupled receptor kinase and (b) further comprises expressing a GRK1 G-protein coupled receptor kinase. In some embodiments, (a) comprises incorporating the polynucleotide into a genome of the cell. In some embodiments, (b) comprises constitutively expressing the polynucleotide. In some embodiments, (b) comprises transiently expressing the polynucleotide. In some embodiments, the cell is a neuronal cell. In some embodiments, the cell is a neuronal stem cell. In some embodiments, the cell is an inner retinal cell. In some embodiments, the inner retinal cell is an ON-bipolar cell, an OFF-bipolar cell, a horizontal cell, a ganglion cell and/or an amacrine cell.

In some embodiments, the cell comprises a cell comprising a retinal degenerative condition. In some embodiments, the retinal degenerative condition is a retinal dystrophy, a rod dystrophy, a rod-cone dystrophy, a cone-rod dystrophy, a cone dystrophy, a macular dystrophy, another form of retinal or macular degeneration, an ischaemic condition, an uveitis or a condition resulting from a loss of photoreceptor function.

Suitably, the method of increasing temporal resolution of an opsin light response, comprising administering to a cell a nucleic acid sequence encoding an opsin comprising a mutation which increases the rate of meta II decay as described above may further comprise administering to the cell a nucleic acid sequence encoding arrestin. Suitably, the nucleic acid encodes wild type arrestin.

Suitably, there is also provided a method of increasing temporal resolution of an opsin light response, comprising administering to a cell an opsin comprising a mutation which increases the rate of meta II decay as described above, and further comprising administering to the cell wild type arrestin.

Suitably, the method comprises contacting a cell comprising the opsin with a suitable composition of the present invention as described herein.

Also disclosed herein is a method of increasing temporal resolution of an opsin light response, comprising administering to a cell a nucleic acid sequence encoding a polynucleotide comprising a nucleic acid sequence encoding the photoactivatable chimeric polypeptide as described herein.

Disclosed herein is a method of increasing a temporal resolution of an opsin light response, comprising contacting the opsin to a G-protein coupled receptor kinase GRK1, or to GRK1 and an arrestin.

### A Method of Providing Photoreceptor Function to a Cell

Also disclosed is a method of providing photoreceptor function to an inner retinal cell, comprising administering an effective amount of a composition comprising a first vector having a first nucleic acid encoding an opsin polypeptide and a second vector having a second nucleic acid encoding a mutant arrestin polypeptide. Also disclosed in some embodiments is a method of providing photoreceptor function to an inner retinal cell, comprising administering an effective amount of a composition comprising a vector comprising a first nucleic acid encoding an opsin polypeptide and a second nucleic acid encoding an arrestin polypeptide. The composition comprising vector(s) may be as described herein.

Disclosed, in some embodiments, is a method of providing photoreceptor function to an inner retinal cell, comprising administering an effective amount of a composition comprising an opsin polypeptide and a phosphorylation independent mutant arrestin polypeptide.

Suitably, the first nucleic acid encoding an opsin polypeptide encodes a meta II decay mutant opsin polypeptide, suitably as described herein.

Suitably, the opsin may be heterologous to the cell, meaning the that opsin is not naturally expressed in the cell but has been introduced into the cell for example by recombinant gene technology.

Disclosed herein is a method of providing photoreceptor function to an inner retinal cell, comprising administering to the cell an opsin comprising a mutation which increases the rate of meta II decay. Suitable mutations may be selected from L59Q, Y74F, E122Q, A132L, A132S, Y136F, I189P, Y227F, Y306F, or a combination thereof. In some embodiments, the mutation may be selected from L59Q, Y74F, E122Q, A132S, Y136F, I189P, Y306F, or a combination thereof. In a suitable embodiment, the mutation may be E122Q.

Also disclosed herein is a method of providing photoreceptor function to an inner retinal cell, comprising administering to a cell a nucleic acid sequence encoding an opsin comprising a mutation which increases the rate of meta II decay. The nucleic acid sequence may encode an opsin having a mutation selected from L59Q, Y74F, E122Q, A132L, A132S, Y136F, I189P, Y227F, Y306F, or a combination thereof. In some embodiments, the nucleic acid sequence may encode an opsin having a mutation selected from L59Q, Y74F, E122Q, A132S, Y136F, I189P, Y306F, or a combination thereof. In a suitable embodiment, the nucleic acid sequence may encode an opsin having an E122Q mutation. The nucleic acid sequence may be provided in a vector, suitably as described herein.

In some embodiments, the cell is a neuronal cell. In some embodiments, the cell is a neuronal stem cell. In some embodiments, the cell is an inner retinal cell. In some embodiments, the inner retinal cell is an ON-bipolar cell, an OFF-bipolar cell, a horizontal cell, a ganglion cell and/or an amacrine cell. Suitably the cell is a human cell.

Suitably, the method of providing photoreceptor function to a cell, comprising administering to a cell a nucleic acid sequence encoding an opsin comprising a mutation which increases the rate of meta II decay as described above may further comprise administering to the cell a nucleic acid sequence encoding arrestin. Suitably, the nucleic acid encodes wild type arrestin.

Suitably, there is also provided a method of providing photoreceptor function to a cell, comprising administering to a cell an opsin comprising a mutation which increases the rate of meta II decay as described above, and further comprising administering to the cell wild type arrestin.

Suitably, the method comprises contacting a cell comprising the opsin with a suitable composition of the present invention as described herein.

Disclosed herein, in some embodiments, is a method for generating photoactivatable cells comprising: (a) delivering a polynucleotide comprising a nucleic acid sequence encoding the photoactivatable chimeric polypeptide to the cell, (b) expressing the photoactivatable chimeric polypeptide. In some embodiments, the polynucleotide of (a) comprises a polynucleotide expression vector comprising the nucleic acid sequence encoding the photoactivatable chimeric polypeptide. In some embodiments, the method (a) further comprises delivering a polynucleotide comprising a nucleic acid sequence encoding a GRK1 G-protein kinase and (b) further comprises expressing the GRK1 G-protein coupled receptor kinase. In some embodiments, (a) comprises incorporating the polynucleotide into a genome of the cell. In some embodiments, (b) comprises constitutively expressing the polynucleotide. In some embodiments, (b) comprises transiently expressing the polynucleotide.

In some embodiments, the cell is a neuronal cell. In some embodiments, the cell is a neuronal stem cell. In some embodiments, the cell is an inner retinal cell. In some embodiments, the inner retinal cell is an ON-bipolar cell, an OFF-bipolar cell, a horizontal cell, a ganglion cell and/or an amacrine cell.

In some embodiments, the cell comprises a cell comprising a retinal degenerative condition. In some embodiments, the retinal degenerative condition is a retinal dystrophy, a rod dystrophy, a rod-cone dystrophy, a cone-rod dystrophy, a cone dystrophy, a macular dystrophy, another form of retinal or macular degeneration, an ischaemic condition, an uveitis or a condition resulting from a loss of photoreceptor function.

### A Method of Treatment

Also disclosed is a method of treating a retinal degenerative condition in a subject in need thereof, comprising administering an effective amount of a composition comprising a first vector having a first nucleic acid encoding an opsin polypeptide and a second vector having a second nucleic acid encoding a mutant arrestin polypeptide. Also disclosed in some embodiments is a method of treating a retinal degenerative condition in a subject in need thereof, comprising administering an effective amount of a composition comprising a vector comprising a first nucleic acid encoding an opsin polypeptide and a second nucleic acid encoding an arrestin polypeptide.

Disclosed, in some embodiments, is a method of treating a retinal degenerative condition in a subject in need thereof, comprising administering an effective amount of a composition comprising an opsin polypeptide and a phosphorylation independent mutant arrestin polypeptide. Also disclosed in some embodiments is an effective amount of a composition comprising an opsin polypeptide and a phosphorylation independent mutant arrestin polypeptide for use in the treatment of a retinal degenerative condition in a subject.

Also disclosed is_an effective amount of a composition comprising a first vector having a first nucleic acid encoding an opsin polypeptide and a second vector having a second nucleic acid encoding a mutant arrestin polypeptide for use in the treatment of a retinal degenerative condition in a subject. Also disclosed is an effective amount of a composition comprising a vector comprising a first nucleic acid encoding an opsin polypeptide and a second nucleic acid encoding an arrestin polypeptide mutant for use in the treatment of a retinal degenerative condition in a subject. The composition comprising vector(s) may be as described herein.

Suitably, a vector may be suitable for targeting expression to an inner retinal cell, most suitably an ON or OFF bipolar cell.

Disclosed herein is a method of treating retinal degenerative condition in a subject in need thereof, comprising administering to the subject an opsin comprising a mutation which increases the rate of meta II decay. Suitable mutations may be selected from L59Q, Y74F, E122Q, A132L, A132S, Y136F, I189P, Y227F, Y306F, or a combination thereof. In some embodiments, the mutation may be selected from L59Q, Y74F, E122Q, A132S, Y136F, I189P, Y306F, or a combination thereof. In a suitable embodiment, the mutation may be E122Q.

Also disclosed herein is a method of treating retinal degenerative condition in a subject in need thereof, comprising administering to the subject a nucleic acid sequence encoding an opsin comprising a mutation which increases the rate of meta II decay. The nucleic acid sequence may encode an opsin having a mutation selected from L59Q, Y74F, E122Q, A132L, A132S, Y136F, I189P, Y227F, Y306F, or a combination thereof. In some embodiments, the nucleic acid sequence may encode an opsin having a mutation selected from L59Q, Y74F, E122Q, A132S, Y136F, I189P, Y306F, or a combination thereof. In a suitable embodiment, the nucleic acid sequence may encode an opsin having an E122Q mutation. The nucleic acid sequence may be provided in a vector, suitably as described herein.

In some embodiments, the retinal degenerative condition is a retinal dystrophy, a rod dystrophy, a rod-cone dystrophy, a cone-rod dystrophy, a cone dystrophy and a macular dystrophy; other forms of retinal or macular degeneration, an ischaemic condition, uveitis or a condition resulting from loss of photoreceptor ability.

In some embodiments, the cell is a neuronal cell. In some embodiments, the cell is a neuronal stem cell. In some embodiments, the cell is an inner retinal cell. In some embodiments, the inner retinal cell is an ON-bipolar cell, an OFF-bipolar cell, a horizontal cell, a ganglion cell and/or an amacrine cell.

In some embodiments, the composition is an injectable liquid. In some embodiments, the composition is administered by injection, preferably an intra-ocular injection, preferably a sub-retinal or intra-vitreal injection.

Suitably, the method of treating a retinal degenerative condition in a subject in need thereof, comprising administering to the subject a nucleic acid sequence encoding an opsin comprising a mutation which increases the rate of meta II decay as described above may further comprise administering to the subject a nucleic acid sequence encoding arrestin. Suitably, the nucleic acid encodes wild type arrestin.

Suitably, there is also provided a method of treating a retinal degenerative condition in a subject in need thereof, comprising administering to the subject an opsin comprising a mutation which increases the rate of meta II decay as described above, and further comprising administering to the subject wild type arrestin.

Suitably, the method comprises contacting a cell in the subject with a suitable composition of the present invention as described herein.

Disclosed is a method of treating a retinal degenerative condition in a subject in need thereof, comprising administering an effective amount of composition comprising a polynucleotide comprising a nucleic acid sequence encoding the photoactivatable chimeric polypeptide. Suitably the method comprises generating photoactivatable cells and/or increasing a temporal resolution of an opsin light response in a cell as described herein.

Also disclosed is an effective amount of a composition comprising a polynucleotide comprising a nucleic acid sequence encoding the photoactivatable chimeric polypeptide for use in the treatment of a retinal degenerative condition in a subject. The treatment may comprise generating photoactivatable cells and/or increasing a temporal resolution of an opsin light response in a cell as described herein.

In some embodiments, the retinal degenerative condition or retinal degeneration is a retinal dystrophy, a rod dystrophy, a rod-cone dystrophy, a cone-rod dystrophy, a cone dystrophy and a macular dystrophy; other forms of retinal or macular degeneration, an ischaemic condition, uveitis or a condition resulting from loss of photoreceptor ability.

Disclosed, herein, is a method of treating a retinal degenerative condition in a subject in need thereof, comprising administering an effective amount of a composition comprising an opsin polypeptide and a G-protein coupled receptor kinase GRK1 polypeptide. Also disclosed in some embodiments is an effective amount of a composition comprising an opsin polypeptide and a G-protein coupled receptor kinase GRK1 polypeptide for use in the treatment of a retinal degenerative condition in a subject. Disclosed, herein, is a method of treating a retinal degenerative condition in a subject in need thereof, comprising administering an effective amount of a composition comprising a first vector having a nucleic acid encoding an opsin peptide, a second vector having a nucleic acid encoding a G-protein coupled receptor kinase GRK1 polypeptide, and a third vector comprising a nucleic acid encoding an arrestin polypeptide. Also disclosed in some embodiments is an effective amount of a composition comprising a first vector having a nucleic acid encoding an opsin peptide, a second vector having a nucleic acid encoding a G-protein coupled receptor kinase GRK1 polypeptide, and a third vector comprising a nucleic acid encoding an arrestin polypeptide for use in the treatment of a retinal degenerative condition in a subject. In some embodiments, the retinal degenerative condition is a retinal dystrophy, a rod dystrophy, a rod-cone dystrophy, a cone-rod dystrophy, a cone dystrophy and a macular dystrophy; other forms of retinal or macular degeneration, an ischaemic condition, uveitis or a condition resulting from loss of photoreceptor ability.

Disclosed herein, in some cases, is a method for generating photoactivatable cells comprising: (a) delivering a polynucleotide comprising a nucleic acid sequence encoding the photoactivatable chimeric, (b) expressing the photoactivatable chimeric. In some cases, the polynucleotide of (a) comprises a polynucleotide expression vector comprising the nucleic acid sequence encoding the photoactivatable chimeric polypeptide. In some cases, the method (a) further comprises delivering a polynucleotide comprising a nucleic acid sequence encoding a GRK1 G-protein coupled receptor kinase and (b) further comprises expressing a GRK1 G-protein coupled receptor kinase. In some cases, (a) is incorporated into a genome of the cell. In some cases, (b) is constitutively expressed. In some cases, (b) is transiently expressed. In some cases, the cell comprises a neuronal cell. In some cases, the cell comprises a neuronal stem cell. In some cases, the cell comprises an inner retinal cell. In some cases, the inner retinal cell is an ON-bipolar cell, an OFF-bipolar cell, a horizontal cell, a ganglion cell and/or an amacrine cell. In some cases, the cell comprises a cell comprising a retinal degenerative condition. In some cases, the retinal degenerative condition is a retinal dystrophy, a rod dystrophy, a rod-cone dystrophy, a cone-rod dystrophy, a cone dystrophy, a macular dystrophy, another form of retinal or macular degeneration, an ischaemic condition, an uveitis or a condition resulting from a loss of photoreceptor function.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1a-1g show the use of Bioluminescence Resonance Energy Transfer (BRET) assay of G protein activation to assess kinetics of opsin-driven light responses in presence of rod photoreceptor deactivation mechanisms: FIG. 1a shows the ratiometric BRET assay of G protein activation; FIG. 1b shows the temporal resolution of rod-opsin driven light responses with BRET assay of G protein activation (BRET Go - Black) is faster than for secondary messenger Glosensor Gso assay (Glo + Gso-Grey); FIG. 1c shows the responses in presence of either G protein receptor kinase 1 (GRK1) and/or visual arrestin (Arr); FIG. 1d shows phosphonull Rod opsin 6A mutant (Rod 6A - unfilled markers) in presence of either G protein receptor kinase 1 (GRK1) and/or visual arrestin (Arr); FIG. 1e shows that the baseline normalised BRET responses are modelled using a simple dual exponential model (left panel) consisting of a scaling factor (A), a one-phase exponential association curve (Rₒₙ) and one-phase exponential decay curve (R_{off}). FIG. 1f shows the relative response amplitude; and FIG. 1g shows the relative response decay, measured as best fit T_{off} (s) for each condition divided by best fit T_{off} for rod opsin positive control (T_{off_}_{RodWT}).

FIGS. 2a-2d show that the phosphorylation-independent arrestin mutants shorten lifetime of rod opsin activity without suppressive effects of GRK1: FIG. 2a shows the time course of BRET light responses for wildtype Rod opsin (RodWT) and FIG. 2b shows the time course of BRET light responses for phosphonull Rod opsin 6A mutant (Rod6A) when co-transfected with either wildtype visual arrestin (ArrWT) or arrestin mutants Arr3A or ArrKEQ3A for unphosphorylated active rod opsin; FIG. 2c shows the response decay (measured as fold change in best fit T_{off}, s) of both RodWT and Rod6A; FIG. 2d shows that the response amplitude of both RodWT and Rod6A is partially increased in presence of ArrWT and Arr3A, but not ArrKEQ3A.

FIGS. 3a and 3b show that the rod opsin-Arrestin fusions have normal subcellular localisation. FIG. 3a shows the diagram of Rod opsin-Arrestin 3A bicistronic and fusion constructs. FIG. 3b shows the heterologous expression of wildtype Rod opsin (Rod WT) only, phosphonull Rod opsin 6A (Rod 6A) only, Rod opsin6A co-expressed with Arrestin 3A (P2A), or Rod opsin 6A tethered to Arrestin 3A by linker in Hek293T cells labelled with anti-rhodopsin 4D2 antibody.

FIGS. 4a-4d show that the rod opsin 6A - Arrestin3A fusions have improved temporal resolution but reduced response amplitude compared to co-expression: FIG. 4a shows the time course of BRET light responses for co-expression or fusions of phosphonull rod opsin mutant (Rod6A) and intermediate affinity arrestin mutant 3A (Arr3A); FIG. 4b shows the response amplitude; FIG. 4c shows the response decay; and FIG. 4d shows comparing response amplitude and decay of Rod6A-Arr3A fusions.

FIGS. 5a-5d show that the rod opsin mutants with increased meta-II decay have variable response amplitude and G protein deactivation: FIG. 5a shows the time course of BRET light responses for rod opsin wildtype (RodWT) and mutants; FIG. 5b shows the response amplitude; FIG. 5c shows the response decay, and FIG. 5d shows comparing relative response amplitude and decay of Rod opsin mutants shows the two parameters are not strongly correlated.

FIGS. 6a-6e show a comparison of response properties of rod opsin wildtype (Rod WT) and phosphonull rod opsin (Rod6A): FIG. 6a shows the time course of BRET response; FIG. 6b shows the response amplitude; FIG. 6c shows the response decay measured as best fit Toff; FIG. 6d shows sensitivity measured as Log EC50, log photons/cm2/s using Glosensor Gso assay for Rod6A and RodWT; FIG. 6e shows the irradiance response curves (IRC) fit to Rod WT or Rod 6A-driven responses.

FIGS. 7a-7f show the modelling Rod opsin-driven BRET responses to different light intensities: FIG. 7a shows that the response decay rate (measured as T_{off}); FIG. 7b shows that the response decay does not significantly correlate with response amplitude; FIG. 7c shows that the response onset is also consistent across intensities; FIG. 7d shows that the response decay does not significantly correlate with response amplitude; FIG. 7e shows that the response amplitude is irradiance-dependent, consistent with increasing levels of G protein activity at higher intensities; and FIG. 7f shows that two parameters, Tₒₙ and T_{off}, are significantly negatively correlated with longer lifetime responses showing shorter response onset.

FIGS. 8a-8d show that the rod opsin 6A - ArrestinWT fusions have reduced response amplitude but faster temporal resolution compared to co-expression: FIG. 8a shows the time course of BRET light responses for co-expression or fusions of phosphonull rod opsin mutant (Rod6A) and wildtype arrestin (ArrWT); FIG. 8b shows the response amplitude; FIG. 8c shows the response decay rate; FIG. 8d shows comparing response amplitude and decay of Rod6A-ArrWT fusions, Rod6A-Arr3A fusion, and Rod6A-10 nm-Arr3A.

FIG. 9 shows the rod opsin meta-II decay mutants have normal subcellular localization in Hek293T cells labelled using anti-rod opsin 1D4 antibody.

FIGS. 10a-10c show no detectable advantage in combining rod opsin meta-II decay mutants with phosphorylation-independent arrestin mutants. FIG. 10a shows the time course of BRET light responses to 1 s 470 nm light (14.1 log photons); FIG. 10b shows the response amplitude; FIG. 10c shows the response decay rate.

FIG. 11 shows a schematic of optogenetic viral transgenes. AAV2 Quad-YF A) Rod6A-10 nm-Arr3A, B) Rod Opsin E122Q and C) Rod Opsin Wildtype. B) and C) are co-expressed with fluorescent mCherry reporter via T2A peptide.

FIG. 12 shows light responses from retinas transduced with Rod6A-10 nm-Arr3A AAV. A) Average firing rate (spikes/s) across light-responsive units. B) Representative responses from individual units. All representative traces show perievent rasters (first trial at top) and associated perievent firing rate histograms (Bin size = 50 ms). Timing of light stimuli shown by horizontal black bar. Error bars show standard error of mean. Stimulus intensity is given in log rod effective photons/cm²/s.

FIG. 13. Light responses from retinas transduced with Rod opsin E122Q AAV. A) Mean firing (spikes/s) across light-responsive units. B) Representative responses from different individual units. .All representative traces show perievent rasters (first trial at top) and associated perievent firing rate histograms (Bin size = 50 ms). Timing of light stimuli shown by horizontal black bar. Error bars show standard error of mean. Stimulus intensity is given in log rod effective photons/cm²/s.

FIG. 14. Expression of mCherry report in retina transduced with Rod opsin E122Q-P2A-mCherry AAV. Retina is displayed bipolar-cell side up - small bright dots represent individually transduced bipolar cells.

FIG. 15. Rod opsin variants show larger and less sustained responses to light compared to native rod opsin. Changes in firing (expressed as standard deviations from baseline mean - z-score) at electrode sites showing a light response are shown for retinas treated with virus driving Rod opsin E122Q (A, n = 6 units, 1 retina) and Rod6A-10 nm-Arr3 (B, 6103, n = 15 units, 3 retinas), with the response in native rod opsin treated retinas (n = 21, 4 retinas) shown in black on each plot for comparison. Note that in all cases, there is a more abrupt and shorter lasting light response in the variants optimised for temporal resolution than in the wild type control. Timing of light stimuli shown by horizontal black bar, data show mean firing. White light stimulus is 15 log rod effective photons/cm²/s

### DETAILED DESCRIPTION

Animal opsins are light activated G protein coupled receptors, suitable for optogenetic control of G protein signalling with high photosensitivity. The lifetime of the photoactivated receptor places a limit on spatiotemporal resolution, which is a particular concern for animal opsins that typically drive slower responses when expressed outside their native environment. Methods of reducing photoresponse duration for a prototypical metazoan opsin (human rod opsin) under heterologous expression in cells, as a step towards improving this aspect of optogenetic control have been achieved. A BRET-based reporter of G protein activation has been used to show that the rod opsin light response lifetime can be shortened by either accelerating decay of meta-II signalling state or, more effectively, enhancing arrestin binding. Phosphorylation-independent arrestin mutants unexpectedly improve signal termination without attenuated amplitude associated with GRK1 expression. Further decrease in response lifetime is possible using opsin-arrestin fusions. Reduction of response lifetime independent of peak response amplitude can be achieved, an important advance in improving animal opsins for optogenetic applications.

In native photoreceptors, signaling of photoactivated opsin is primarily quenched by interaction with arrestin. As arrestin binding requires receptor phosphorylation, this mode of deactivation requires both arrestin and a suitable G protein receptor kinase. In optogenetic applications, one or both may be absent or insufficiently abundant in the host cell. Some opsins also have an intrinsic partial deactivation mechanism in the form of hydrolysis of the Schiff base linkage that binds the agonist, all-trans form of retinal, to opsin apoprotein. This leads to decay of the signalling active ‘meta II’ state of opsin. Reducing opsin photoresponse duration may be achieved by minimizing the signaling-active lifetime of photoactivated animal opsins under heterologous expression, such as increasing the rate of Schiff-base hydrolysis or allowing enhanced arrestin binding.

The present invention is, in part, based on the discovery that opsin-driven responses can be rendered more time-delimited with manipulations designed to enhance either arrestin-opsin interactions or the decay rate of the signalling-active meta-II opsin state. Applying arrestin mutants with enhanced affinity for unphosphorylated active receptor effectively reduced response lifetime in the absence of GRK1, avoiding the suppressive effects of GRK1 expression on response amplitude. Application of the arrestin mutants brings the additional benefit of simplicity. For optogenetic applications, in which packaging size for vectors is often limiting, introducing two proteins (opsin+arrestin) is much more feasible than introducing three (opsin+arrestin+kinase).

It was surprising that tethering a mutant arrestin (e.g., Arr3A) to an opsin protein allowed faster response decay substantially without affecting, i.e. reducing compared to wild type, amplitude response. This tethering strategy can also mitigate potential problems associated with employing the phosphorylation-independent arrestin mutants. It was surprising that tethering arrestin to opsin did not cause opsin to lose its function. Indeed, tethering arrestin to opsin helps reduce the potential of these introduced arrestins to interfere with native G-protein-coupled receptors and their separate signalling cascades. In addition, tethering the arrestin to an opsin such as rod opsin could limit the off-target effects of arrestin (e.g., arr3A) and ameliorate potential cytotoxicity. Overexpression of Arr3A mutant in mouse rods caused photoreceptor degeneration indicating that it may be cytotoxic; the deleterious effects of this mutant in photoreceptors is believed to be due to reduced self-association of Arr3A, leading to high concentration of monomer units which interact with signalling pathways that can cause apoptosis. The cytotoxicity of these mutants may be unique to rod photoreceptors, which have unique morphology and protein expression (possessing 1000-10000 more signalling proteins). Tethering a mutant arrestin (e.g., Arr3A) to the opsin may help prevent or reduce cytotoxicity.

Altering the opsin protein to increase the rate of meta-II decay can help reduce response lifetime. The experiments described herein showed the surprising results that the increased rate of meta-II decay translates into faster termination of G protein response to light flash. A variety of rod opsin mutants thought to destabilise the Schiff base linking the retinaldehyde chromophore to the opsin protein moiety and increase the rate of Schiff base hydrolysis in the G protein signalling meta-II state can be applied. A range of opsin mutants had previously been characterised using spectroscopic methods and were reported to cause a variety of changes to the rate of Rod WT meta-II decay, from relatively small 1.2-5 fold increases of Y74F, Y136F and Y306F to more dramatic 4-8 fold change of A132L, E122Q and Y223F. When modulating signalling lifetime for animal opsins in live cell environments, both arrestin interaction and meta-II decay are suitable targets for modulation.

Adapting animal opsins for optogenetic applications can be used herein. Rod opsin is the most extensively characterized of all opsins, and its deactivation by both kinase/arrestin and Schiff-base hydrolysis mechanisms very well established. Human rod opsin is also a potentially important optogenetic tool, suitable for an application in which spatiotemporal resolution is particularly important. It is a human protein that expresses well ectopically, is highly sensitive and capable of coupling to native Gi/o/t pathways. Rod opsin can be expressed in the surviving cells of retinal degenerate mice to restore basic image-forming vision at physiological light intensities. This reduced temporal resolution could also affect spatial resolution causing blurring due to head and eye movement, issues detecting motion and problems adapting to changes in light levels.

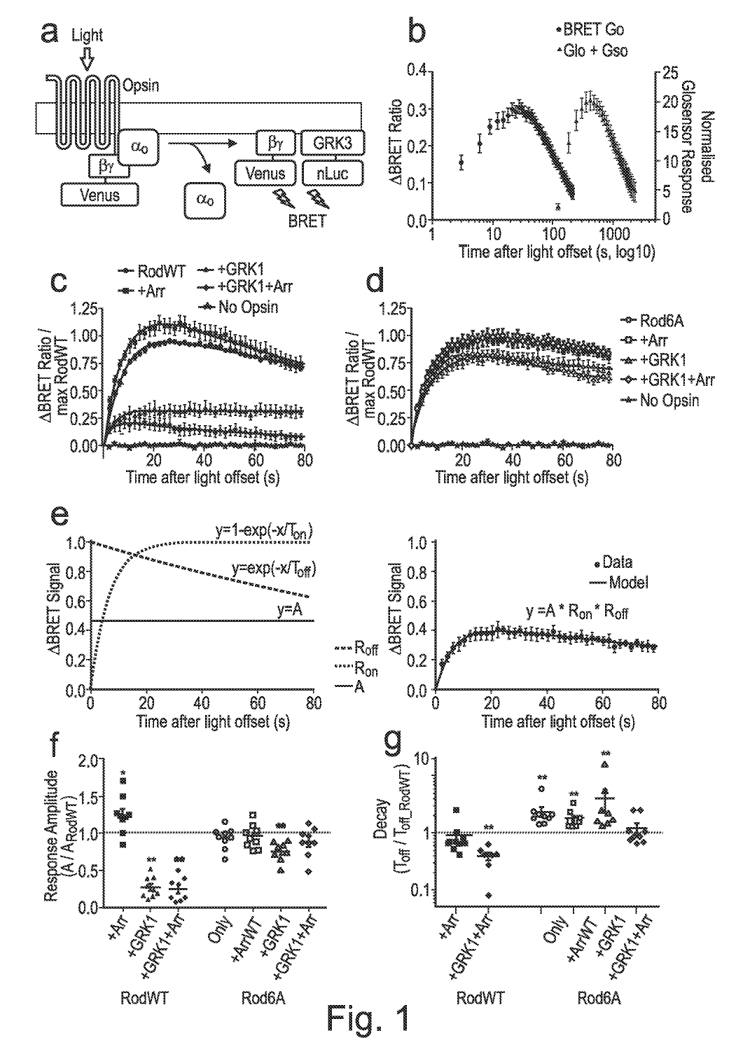

A method of determining the lifetime of photoactivated opsin is also described herein. Although this can be achieved using in vitro preparations of purified protein, here rod opsin was tested in a live cell environment more directly relevant for optogenetic applications. To this end, we used a BRET-based assay to provide near real-time readout of G-protein activation as described in Masuho et al, Science Signaling 01 Dec. 2015: Vol. 8, Issue 405, pp. ra123], which includes a schematic view of the ratiometric BRET assay of G protein activation as shown in FIG. 1a. A wide array of interventions, aiming to either enhance arrestin binding or accelerate Schiff base hydrolysis, can reduce the lifetime of the photoresponse. Such manipulations tend also to reduce peak response amplitude but, as there is no simple relationship between the magnitude of these two effects, these appear to be at least partially separable phenomena. Described herein are strategies for increasing the temporal fidelity of the rod opsin light response under heterologous expression while minimising the impact on response amplitude. Recognized herein is a need for compositions, systems, and methods for engineering human photoactive GPCR molecules so that they can be utilized for the development of improved optogenetic applications. The present disclosure provides engineered photoactive chimeric polypeptides and compositions that enable efficient spatiotemporal activation of photoactive GPCR signaling. The present disclosure provides compositions, systems, and methods that yield efficient spatiotemporal activation by efficiently activating G-protein signaling in a manner that produces a high sensitivity and without loss (attenuation) of response amplitude. The present disclosure also provides compositions, systems, and methods that yield efficient spatiotemporal activation by reducing the G-protein signaling activation lifetime in a manner that results a high spatiotemporal resolution. Furthermore, the present disclosure provides compositions, systems, and methods wherein the spatiotemporal modulation of opsin signaling is achieved in the context of human opsin polypeptide.

The present inventors have shown that the compositions, kits and methods described herein enable the detection of light responses at commonly encountered light levels (13.5-15.5 log photons/cm2/s) in degenerate retina.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Herein the embodiments described in relation to an aspect of the invention may apply mutatis mutandis to any of the other aspects of the invention.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Definitions

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

By “operably linked” means that the nucleic acid sequence or polypeptide is functionally associated with the sequence to which it is operably linked, such that they are linked in a manner such that they affect the expression or function of one another. For example, a polypeptide may alter the activity or signaling cascade of another polypeptide to which it is operably linked. For example, a nucleic acid sequence operably linked to a promoter will have an expression pattern influenced by the promoter. Rod opsin and arrestin polypeptide may be operably linked by way of a linker. A linker may be selected from the group consisting of a flexible linker, a rigid linker, a semi-flexible linker, ER/K linker, or a combination thereof. In some embodiments, the linker is a flexible glycine-serine linker, a rigid alpha-helix forming linker, a semi-flexible linker having a rigid linker with flexible ends, and a ER/K linker, or a combination thereof. A linker may be naturally occurring or non-naturally occurring.

A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm flexible ER/K semi-flexible linker.

A promoter mediates expression of the nucleic acid sequence to which it is linked. A promoter may be constitutive or may be inducible. A promoter may direct ubiquitous expression in the inner retinal cells, or neurone specific expression. In the latter case, a promoter may direct cell type specific expression, for example to ON bipolar or OFF bipolar cells. Suitable promoters will be known to persons skilled in the art. For example, a suitable promoter for use in the present invention may be selected from the group consisting of L7, thy- 1 , recoverin, calbindin, human CMV, GAD-67, chicken beta-actin, hSyn, Grm6, Grm6 enhancer-SV40 fusion protein. Targeting may be achieved using cell specific promoters, for example e.g. Grm6-SV40 for selective targeting of ON-bipolar cells. The Grm6 promoter is a fusion of 200-base pair enhancer sequence of the Grm6 gene encoding for ON-bipolar cell specific metabotropic glutamate receptor, mGluR6, and an SV40 eukaryotic promoter. Preferred sources of the Grm6 gene are mouse and human. Ubiquitous expression may be achieved using a pan-neuronal promoter, examples of which are known and available in the art. One such example is CAG. The CAG promoter is a fusion of CMV early enhancer and chicken β-actin promoter.

“Opsin” and “opsin polypeptide,” as used herein, refer to a naturally occurring opsin polypeptide from any species, biologically active fragment of opsin, as well as any variant, equivalent, or homologous or mutant forms thereof. An opsin includes, for example, a rod opsin (rhodopsin), cone opsins such as blue, green and red pigments, (Nathans, J., Annu. Rev. Neurosci. 10:163-194 (1987)), and their biologically active fragments. In addition, for example, opsins include melanopsin (Provencio et al., J. Neurosci. 20:600-605(2000)), encephalopsin or panopsin (Blackshaw and Snyder J. Neurosci. 19:3681-3690 (1999); Halford et al., Genomics 72:203-208 (2001)) and peropsin (Sun et al., Proc. Natl. Acad. Sci. USA 94:9893-9898 (1997)), neuropsin (Opn5), (Opn3), a parapineal opsin, VAopsin, parapinopsin; parietopsin, pinopsin, TMT opsin, Jelly fish opsin, C-opsin, and any invertebrate retinal opsins and/or opsins normally supporting extra- retinal photosensitivity in animals. A mutant opsin may have any one or more mutations as described herein, A wild type opsin as referred to herein may have the sequence of NCBI Reference Sequence NM_000539 version 3, or may be a variant thereof which does not comprise any one or more of the opsin mutations as described herein. Herein, rod opsin may include rod opsin mutated to remove C-terminal phosphorylation sites. Suitably, the mutations are S333A, T336A, S338A, T340A, T342A, S343A of human rod opsin (referred to as Rod6A). Herein, an opsin segment includes opsin as defined herein, or any biologically active fragments thereof.

“Arrestin” or “arrestin polypeptide,” as used herein, refers to a naturally occurring arrestin polypeptide from any species, biologically active fragments, as well as any variant, equivalent, or homologous or mutant forms thereof. Examples of arrestin include but are not limited to visual arrestin (sometimes referred to as Arrestin 1), βarrestin 1 (sometimes referred to as Arrestin 2), βarrestin 2 (sometimes referred to as Arrestin 3), and cone arrestin. A mutant arrestin may have any one or more mutations as described herein, A wild type arrestin as referred to herein may have the sequence of NCBI Reference sequence NM_000541, or may be a variant thereof which does not comprise any one or more of the arrestin mutations as described herein. A mutant arrestin includes a phosphorylation independent arrestin, which means that it has a mutation associated with increased affinity for unphosphorylated opsin. The residue numbering for the arrestin point mutations defined herein is made with reference to nucleotide 1 being 236 of the NCBI Reference sequence NM_000541.

“G protein coupled receptor kinase” refers to a naturally occurring G protein coupled receptor kinase from any species, biologically active fragments, as well as any variant, equivalent, or homologous forms thereof. A suitable example includes GRK1 (G protein coupled receptor kinase 1, or rhodopsin kinase).

“Polypeptide Composition”: A composition described herein can be useful for modulating the spatiotemporal activation and deactivation of G-protein signaling. A composition described herein can also be useful for modulating the spatiotemporal activation in optogenetic applications, systems, and technologies. The compositions described can achieve spatiotemporal modulation through a mechanism comprising modulating the photoactivity lifetime. Described herein are compositions that can modulate the spatiotemporal activation and deactivation of GPCRs by modulating the intrinsic photoactivity of GPCRs or by modulating photoactivity through GPCR-binding molecules that inhibit and/or quench a photoactive state.

A composition described herein can utilize GPCR-binding polypeptides to modulate the spatiotemporal activation of photoactive GPCRs in response to light. A GPCR-binding polypeptide can quench an activated and subsequently modulate photoactive GPCR signaling by modulating the amplitude of a GPCR signal and altering the longevity of a response. Modulation by GPCR-binding polypeptides typically requires the presence of a G-protein specific protease and site-specific GPCR-phosphorylation. The composition described herein can comprise a photoactive GPCR and a GPCR-binding polypeptide capable of binding the GPCR independent of GPCR phosphorylation and modulating GPCR activity. The composition described herein can also utilize mutation within a photoactive GPCR to modulate the spatiotemporal activation of G-protein signaling in response to light. A GPCR mutation can modulate photoactive GPCR signaling by modulating the amplitude of a GPCR signal and altering the longevity of a response. Mutations within a photoactive GPCR can modulate the amplitude of a GPCR signal and altering the longevity of a response through the stabilization of an inactive state or destabilization of an active state. In some cases, the photoactive GPCR is opsin and the GPCR-binding polypeptide is arrestin.

In some cases, the opsin comprises a mutation associated with a C-terminal phosphorylation site. In some cases, the arrestin comprises a mutation associated with increased affinity for unphosphorylated opsin. In some cases, the mutation is selected from L337A, V378A, F379A, K261Q, E350H, Q332K, or a combination thereof. In some cases, mutation comprises L337A, V378A, and F379A. In some cases, the mutation comprises L337A, V378A, F379A, K261Q, E350H, and Q332K. In some cases, the opsin comprises at least one mutation that modulates spatiotemporal activation of the opsin polypeptide. In some cases, the mutation is selected from L59Q, Y74F, E122Q, A132L, A132S, Y136F, I189P, Y227F, Y306F, or a combination thereof. In some cases, the mutation is selected from L59Q, Y74F, E122Q, A132S, Y136F, I189P, Y306F, or a combination thereof. In some embodiments, the arrestin comprises a mutation associated with affinity for unphosphorylated opsin. In some embodiments, the arrestin polypeptide comprises a mutation selected from L337A, V378A, F379A, K261Q, E350H, Q332K, or a combination thereof. In some embodiments, the arrestin polypeptide comprises a mutation selected from L337A, V378A, F379A, and any combination thereof. In some embodiments, the arrestin polypeptide comprises a mutation selected from L337A, V378A, F379A, K261Q, E350H, Q332K, and any combination thereof. In some embodiments, the opsin comprises a mutation associated with a C-terminal phosphorylation site and the arrestin comprises a mutation associated with affinity for unphosphorylated opsin. In some embodiments, the opsin comprises a mutation selected from L59Q, Y74F, E122Q, A132L, A132S, Y136F, I189P, Y227F, Y306F, or a combination thereof; and the arrestin comprises a mutation selected from L337A, V378A, F379A, K261Q, E350H, Q332K, or a combination thereof. In some embodiments, the combination of mutations may comprise any one or more mutations selected from L59Q, Y74F, E122Q, A132L, A132S, Y136F, I189P, Y227F, Y306F and any one or more mutations selected from L337A, V378A, F379A, K261Q, E350H, Q332K. In some embodiments, the opsin comprises a mutation associated with a C-terminal phosphorylation site and/or a mutation which increases the rate of meta II decay, and the arrestin comprises a mutation associated with affinity for unphosphorylated opsin. In some embodiments, the opsin comprises a mutation selected from mutant associated with a C terminal phosphorylation site, for example S333A, T336A, S338A, T340A, T342A, S343A, or any combination thereof and/or a mutation which increases the rate of meta II decay, for example selected from L59Q, Y74F, E122Q, A132L, A132S, Y136F, I189P, Y227F, Y306F, or a combination thereof; and the arrestin comprises a mutation selected from L337A, V378A, F379A, K261Q, E350H, Q332K, or a combination thereof. In some embodiments, the combination of mutations may comprise a mutation selected from a) and/or b), and/or c), wherein a) comprises S333A, T336A, S338A, T340A, T342A, S343A; b) comprises L59Q, Y74F, E122Q, A132L, A132S, Y136F, I189P, Y227F, Y306F or a combination thereof and c) comprises L337A, V378A, F379A, K261Q, E350H, Q332K or a combination thereof. In a suitable embodiment, an opsin mutation is E122Q, in combination with wild type arrestin. In a suitable embodiment, an opsin mutation is E122Q, in combination with an arrestin mutation at L337A, V378A and F379A (referred herein as 3A). In a suitable embodiment, the opsin mutation is E122Q, S333A, T336A, S338A, T340A, T342A, and S343A in combination with an arrestin mutation at L337A, V378A, and F379A (3A). In a suitable embodiment the opsin is human rod opsin and the mutation is E122Q, S333A, T336A, S338A, T340A, T342A, and S343A (E122Q rod 6A) in combination with an arrestin mutation at L337A, V378A, and F379A (3A).

In some cases, the opsin is an animal opsin. In some cases, the opsin is a human rod opsin. In some cases, a composition is suitable for ocular or subretinal administration.

A composition described herein can utilize G-protein coupled receptor specific kinases to modulate the amplitude and duration of a photoactive response to light. A G-protein coupled receptor kinase can phosphorylate specific residues on a GPCR that can result in reducing the amplitude of a GPCR signal and altering the longevity of a response. The composition can then comprise a photoactivatable GPCR and a G-protein-specific kinase.

Disclosed herein, in some cases, is a composition comprising an opsin polypeptide and a G-protein coupled receptor kinase GRK1 polypeptide. In some cases, the opsin polypeptide is operably linked to the mutant G-protein coupled receptor kinase GRK1 polypeptide through a linker. In some cases, the linker is a flexible linker, a rigid linker, a semi-flexible linker, a ER/K linker, or a combination thereof. In some cases, the linker is a flexible glycine-serine linker, a rigid alpha-helix forming linkers, a semi-flexible linker having a rigid linker with flexible ends, and a ER/K linker, or a combination thereof. A linker may be naturally occurring or non-naturally occurring. In some cases, the opsin polypeptide and the mutant G-protein coupled receptor kinase GRK1 polypeptide are separate. A linker may be 5-250 amino acids in length, more suitably 8 to 150, more suitably 8-100 amino acids, most suitably 10-100. A most suitable linker may be 8-12, suitably 10 amino acids in length. A suitable linker may be a 10 nm ER/K semi-flexible linker.

A composition may comprise a vector or polypeptide as described herein, together with one or more suitable vehicles or excipients, for example for administration to a cell. In some cases, a composition is an injectable liquid. In some cases, the composition is administered by injection, preferably an intra-ocular injection, preferably a sub-retinal or intra-vitreal injection.

By increasing temporal resolution is meant improving the response of the cell to light, compared to a non-transformed cell. For example, a cell with increased temporal resolution may exhibit faster opsin activation and/or faster opsin deactivation compared to a native or non-transformed cell.

By providing photoreceptor function is meant that a cell which previously did not have photoreceptor ability or whose photoreceptor ability has degenerated, wholly or partially, becomes photo-receptive upon expression therein of the foreign nucleic acid sequence encoding a photosensitive protein. Preferably, a transformed retinal cell exhibits some or all of the photoreceptor ability of a native photoreceptive cell. Preferably, a transformed cell exhibits at least the same or substantially the same photoreceptive ability of a native retinal photoreceptor cell. Preferably, a transformed cell exhibits higher photoreceptive ability than a diseased or degenerating native retinal photoreceptor cell. Therefore, a transformed cell will preferably have increased photoreceptor compared to a degenerated or diseased cell from the same source, maintained under the same conditions, without treatment. A transformed cell can be distinguished from a native cell by the presence therein of exogenous nucleic acid. By “restoring photoreceptor function” may, in some embodiments, mean that a light response can be detected at a commonly encountered light levels (for example 13.5-15.5 log photons/cm2/s) in degenerate retina.

By retinal degeneration or a retinal degenerative condition or disease is meant any condition which results in loss of photoreceptor function in the cell of the retina, or loss of cells in the retina. Retinal degeneration may result in partial or complete loss of vision.

A recombinant cell herein is a cell to which a vector or nucleic acid as described herein has been administered and taken up by the cell. Such a cell may be referred to herein as a transformed cell, because it comprises therein non-native nucleic acid. The vector or nucleic acid may be described as being foreign, non-native or heterologous to the cell.

Disclosed herein, in some cases, is a method of increasing deactivation of an opsin, comprising contacting the opsin to a G-protein coupled receptor kinase GRK1, contacting the opsin to the arrestin, or contacting the opsin to a G-protein coupled receptor kinase GRK1 and an arrestin. Disclosed herein, in some cases, is a method of increasing a temporal resolution of an opsin light response, comprising contacting the opsin to a G-protein coupled receptor kinase GRK1, contacting the opsin to the arrestin, or contacting the opsin to a G-protein coupled receptor kinase GRK1 and an arrestin. Disclosed herein, in some cases, is a method of providing photoreceptor function to an inner retinal cell, comprising administering to an inner retinal cell an effective amount of a composition comprising an opsin, a G-protein coupled receptor kinase GRK1, and an arrestin.

When deactivation is increased, the total duration of G protein signaling is shorter and the response decay occurs faster after cessation of light stimulus than in an unmodified cell or system.

Photoactive Polypeptide: The photoactive polypeptide described herein can be useful for modulating the spatiotemporal activation and deactivation of G-protein signaling. The photoactive polypeptide described herein can also be useful for modulating the spatiotemporal activation in optogenetic applications, systems, and technologies. The photoactive polypeptide described can achieve spatiotemporal modulation through a mechanism comprising modulating the photoactivity lifetime. Described herein are photoactive polypeptides that can modulate the spatiotemporal activation and deactivation of GPCRs by modulating the intrinsic photoactivity of GPCRs or by modulating photoactivity through GPCR-binding molecules that inhibit and/or quench a photoactive state. The photoactive polypeptide may be an opsin, suitably an animal opsin, most suitably a human opsin. The opsin may be rod opsin, suitably rod opsin 6A, or may be a meta II decay mutant as described herein. In some embodiments, the photoactive polypeptide can be a chimeric polypeptide, for example as described below.

The photoactive polypeptide described herein can utilize GPCR-binding polypeptides to modulate the spatiotemporal activation of photoactive GPCRs in response to light. A GPCR-binding polypeptide can quench an activated and subsequently modulate photoactive GPCR signaling by modulating the amplitude of a GPCR signal and altering the longevity of a response. Modulation by GPCR-binding polypeptides typically requires the presence of a G-protein specific protease and site-specific GPCR-phosphorylation. The photoactive chimeric polypeptide described herein can comprise a photoactive GPCR and a GPCR-binding polypeptide capable of binding the GPCR independent of GPCR phosphorylation and modulating GPCR activity. The photoactive chimeric polypeptide described herein can also utilize mutation within a photoactive GPCR to modulate the spatiotemporal activation of G-protein signaling in response to light. A GPCR mutation can modulate photoactive GPCR signaling by modulating the amplitude of a GPCR signal and altering the longevity of a response. Mutations within a photoactive GPCR can modulate the amplitude of a GPCR signal and altering the longevity of a response through the stabilization of an inactive state or destabilization of an active state.

By a modulatory segment is meant any polypeptide or fragment, variant or mutant thereof which is capable of modulating the activity of an opsin, more suitably capable of modulating the ability of an opsin to initiate a downstream signaling cascade via transducin. Most suitably a modulatory segment is capable of deactivating or inhibiting the ability of an opsin to activate G protein.

A photosensitive polypeptide is one which reacts to light, by undergoing a chemical or physical change. By photoreceptive, means a cell which is photosensitive or comprises one or more photosensitive proteins. The terms photoreceptive or photoreceptor and photosensitive may be used interchangeably. A nucleic acid sequence for use in the invention may encode any photosensitive polypeptide. Preferably, the nucleic acid sequence of the invention encodes a mammalian or non-mammalian photosensitive protein. It may be mammalian, non-mammalian, plant, bacterial, or archeabacterial in origin. Where mammalian, it is preferred that it encodes a human protein. A nucleic acid sequence for use in the present invention may be selected from the group consisting of rhodopsin, melanopsin, a cone opsin (in particular LWS opsin, MW opsin, and SWS opsin), neuropsin (Opn5), encaphalopsin (Opn3), a parapineal opsin, VAopsin, parapinopsin; parietopsin, pinopsin, TMT opsin, Jelly fish opsin, C-opsin, and any invertebrate retinal opsins and/or opsins normally supporting extra-retinal photosensitivity in animals.

By photoactive is meant activity driven by photosensitive polypeptide in response to light. “Photoactivable” means that the cell is made capable of generating activity in response to light. These terms may be used interchangeably with photosensitive or photoreceptive under the general meaning of responsive to light.

Gene therapy and vector: The terms photoreceptive or photoreceptor and photosensitive may be used interchangeably. A nucleic acid sequence for use in the invention may encode any photosensitive protein. Preferably, the nucleic acid sequence of the invention encodes a mammalian or non-mammalian photosensitive protein. It may be mammalian, non-mammalian, plant, bacterial, or archeabacterial in origin. Where mammalian, it is preferred that it encodes a human protein. A nucleic acid sequence for opsin in the present invention may be selected from the group consisting of rhodopsin, melanopsin, a cone opsin (in particular LWS opsin, MW opsin, and SWS opsin), neuropsin (Opn5), encaphalopsin (Opn3), a parapineal opsin, VAopsin, parapinopsin; parietopsin, pinopsin, TMT opsin, Jelly fish opsin, C-opsin, and any invertebrate retinal opsins and/or opsins normally supporting extra- retinal photosensitivity in animals. A nucleic acid sequence for arrestin in the present invention may be selected from the group consisting of Arrestin-1, Arrestin-2, Arrestin-3, and Arrestin-4.

A nucleic acid sequence for use in the present invention may be selected depending upon the subject to be treated, such that the nucleic acid sequence encodes a photosensitive protein which is native to the retina of the subject to be treated. Thus, for example, where the subject is a human, a nucleic acid sequence will preferably encode a human photosensitive protein, for example rhodopsin. However, it is envisaged that in certain embodiments, a nucleic acid sequence may be provided which encodes a photosensitive protein which is not native to the subject to be treated, but which preferably does not raise an immune response in the subject.

The nucleic acid sequences and amino acid sequences of many photosensitive proteins are known in the art. For example, the nucleic acid sequences of preferred photosensitive proteins are provided as follows:
Melanopsin: Homo sapiens opsin 4 (OPN4), mRNA (cDNA clone MGC: 142118 IMAGE:8322610), GenBank: BC113558, Version BC1 13558.1 ;
Rhodopsin: Homo sapiens rhodopsin (RHO), GenBank: BC1 11451.3, Accession NM_000539, Version NM_000539.3 Gl: 169808383;
Cone homo sapiens opsin 1 : Homo sapiens opsin 1 , long-wave sensitive, OPN1 LW - NCBI Reference Sequence: Accession: NM_020061 , Version NM_020061.5;
Homo sapiens opsin 1 , medium-wave sensitive OPN1 MW - NM_000513, version NM_000513.2;
Homo sapiens opsin 1 short-wave-sensitive (OPN1SW) NM_001708, version NM_001708.2. Parapinopsin (Genbank Accession NM_001200073, Version NM_001200073.1 Gl:318056020);
Parietopsin (Genbank Accession DQ100320, Version DQ100320.1 Gl:73666459); Pinopsin (Genbank Accession AF487546, Version AF487546.1 Gl:20805654); VA opsin (Genbank Accession AF233520, Version AF233520.1 Gl:8272567);
TMT opsin (Genbank Accessions AH011520 AF349943 AF349944 AF349945, version AH011520.2 Gl:33951 1123);
Jelly fish opsin (Genbank Accession AB435549, Version AB435549.1 G 1:210049957); OPN3 (Genbank Accession NM_014322, Version NM_014322.2 Gl:71999130);
OPN5 (Genbank Accession AY377391 , Version AY377391.1 Gl:38482095);
C-opsin (Genbank Accession HF566407, version HF566407.1 Gl: 543581059); and Cryptochrome (Genbank Accession NM_169852, Version NM_169852.1 Gl:24648151).

The photoactive or photosensitive protein referred to herein may be a human protein. A human photoactive or photosensitive protein may be human Rhodopsin (also referred to as Rh1, OPN2, RHO) or a photopsin. A photopsin may be selected from the group consisting of Long Wavelength Sensitive (OPN1 LW) Opsin, Middle Wavelength Sensitive (OPN1 MW) Opsin and Short Wavelength Sensitive (OPN1 SW) Opsin. Long Wavelength Sensitive (OPN1 LW) Opsin has an Amax of 560 nm, in the yellow-green region of the electromagnetic spectrum. It is also referred to as “red opsin”, “L opsin” or “LWS opsin”. Middle Wavelength Sensitive (OPN1 MW) Opsin has a Amax of 530 nm, in the green region of the electromagnetic spectrum. It is also referred to as the “green opsin”, “M opsin” or “MWS opsin”. Short Wavelength Sensitive (OPN1SW) Opsin has a Amax of 430 nm, in the blue region of the electromagnetic spectrum. It is also referred to as the “blue opsin”, “S opsin” or “SWS opsin”.

The nucleic acid sequence encoding a human photoactive or photosensitive protein may be the Homo sapiens rhodopsin (RHO) gene (GenBank: BC1 11451.3, Accession NM_000539, Version NM_000539.3 Gl: 169808383), or a fragment or derivative thereof.

The nucleic acid sequence encoding a human photoactive or photosensitive protein may be the Cone homo sapiens opsin 1, long wave sensitive OPN1 LW (NCBI Reference Sequence: Accession: NM_020061, Version NM_020061.5), or a fragment or derivative thereof. The nucleic acid sequence encoding a human photoactive or photosensitive protein may be the Cone homo sapiens opsin 1: medium-wave sensitive OPN1 MW, (NCBI Reference Sequence: Accession: NM_000513.2; (Science 232 (4747), 193-202 (1986)), or a fragment or derivative thereof. The nucleic acid sequence encoding a human photoactive or photosensitive protein may be the Cone homo sapiens opsin 1: short-wave-sensitive (OPN1 SW) NM_001708, version NM_001708.2, or a fragment or derivative thereof. Reference to a nucleic acid sequence encoding a photosensitive protein includes nucleic acid sequences which are derivatives of the sequences described herein, or encode a shorter version, or a fragment of a photosensitive protein, wherein the derivative or fragment retains substantially the same photosensitive function as the native photosensitive protein. By substantially the same is meant at least 70%, 75%, 80%, 85%, 90%, 95%, 96%, 97%, 98%, or 99% of the photosensitive function of the native protein. A fragment may comprise 70%, 75%, 80%, 85%, 90%, 95%, 96%, 97%, 98%, or 99% of the sequence of the native protein.

referably a fragment or derivative of a nucleic acid sequence shares at least 70%, 75%, 80%, 85% or 90%, at least 91, 92, 93, 94, 95, 96, 97, 98, or at least 99% sequence identity with a reference nucleic acid sequence, over a length of 50%, 60%, 70%, 80%, 90%, or at least 95% of the length of a reference nucleic acid sequence. A derivative is preferably active and may include substitutions and/or deletions and/or additions compared to the native sequence. Derivatives may also include portions of other gene sequences, which provide a desired activity or function to the photosensitive protein. A derivative may also be referred to as a mutant or variant of the reference sequence.

Sequence identity is determined by comparing the two aligned sequences over a predetermined comparison window (which may be 50%, 60%, 70%, 80%, 90%, 95%, or 100% of the length of the reference nucleotide sequence or protein), and determining the number of positions at which identical residues occur. Typically, this is expressed as a percentage. The measurement of sequence identity of a nucleotide sequences is a method well known to those skilled in the art, using computer implemented mathematical algorithms such as ALIGN (Version 2.0), GAP, BESTFIT, BLAST (Altschul et al J. Mol. Biol. 215: 403 (1990)), FASTA and TFASTA (Wisconsin Genetic Software Package Version 8, available from Genetics Computer Group, Accelrys Inc. San Diego, California), and CLUSTAL (Higgins et al, Gene 73: 237-244 (1998)), using default parameters.

A nucleic acid sequence may be a DNA, RNA, cDNA, or PNA. It may be genomic, recombinant or synthetic. A nucleic acid sequence may be isolated or purified. It may be single stranded or double stranded. Preferably, a nucleic acid sequence will encode a photosensitive protein, as described herein. A nucleic acid sequence may be derived by cloning, for example using standard molecular cloning techniques including restriction digestion, ligation, gel electrophoresis, for example as described in Sambrook et al; Molecular Cloning: A laboratory manual, Cold Spring Harbour laboratory Press). A nucleic acid sequence may be isolated, for example using PCR technology. Such technology may employ primers based upon the sequence of the nucleic acid sequence to be amplified. By isolated is meant that the nucleic acid sequence is separated from any impurities and from other nucleic acid sequences and/or proteins which are naturally found associated with the nucleic acid sequence in its source. Therefore, it may be separated from flanking nucleic acid sequences, or from chromosomal material or sequence. Preferably, it will also be free of cellular material, culture medium, or other chemicals from a purification/production process. A nucleic acid sequence may be synthetic, for example produced by direct chemical synthesis e.g. using the phosphotriester method (Narang et al Meth Enzymol 68: 109-151 1979). A nucleic acid sequence may be provided as naked nucleic acid, or may be provided complexed with a protein or lipid. The sequence may be altered to improve expression efficiency (for example by truncating C-terminus or introducing targeting motifs), or to alter characteristics of the light response (for example by removing or adding residues targeted by rhodopsin kinases as part of the signal termination process). With the sequence information provided, the skilled person can use available cloning techniques to produce a nucleic acid sequence or vector suitable for transduction into a cell.

Preferably, a nucleic acid sequence is provided as a vector, preferably an expression vector. Preferably, it may be provided as a gene therapy vector, preferably which is suitable for transduction and expression in a target retinal cell. A vector may be viral or non-viral (e.g. a plasmid). Viral vectors include those derived from adenovirus, adenoassociated virus (AAV) including mutated forms, retrovirus, lentivirus, herpes virus, vaccinia virus, MMLV, GaLV, Simian Immune Deficiency Virus (SIV), HIV, pox virus, and SV40. A viral vector is preferably replication defective, although it is envisaged that it may be replication deficient, replication competent or conditional. A viral vector may typically persist in an extrachromosomal state without integrating into the genome of the target retinal cell. A preferred viral vector for introduction of a nucleic acid sequence encoding a photosensitive protein to a retinal target cell is an AAV vector, for example self-complementary adenoassociated virus (scAAV). Selective targeting may be achieved using a specific AAV serotype (AAV serotype 1 to AAV serotype 12), particularly AAV2 or a modified version of any of these serotypes including modified versions of AAV2 such as AAV 4YF and AAV 7m8 vectors. In aspects of the invention where the vector is provided by intra-vitreous administration, the vector may be one which has been modified such that it does not bind to one or more proteins of the ECM. For example, a preferred vector may comprise a modified heparin sulphate binding site, such that it has reduced or an inability to bind heperan sulphate, such as AAV 7m8 (Dalkara D et al Sci Transl Med 2013; 5: 189ra76). In some embodiments, the AAV vector is selected from AAV1-AAV10. Suitably, the AAV vector is AAV2.

A vector may comprise one nucleic acid sequence selected from a nucleic acid sequence encoding rod opsin or arrestin, as described herein. A vector may be bicistronic, meaning that it encodes two or more genes, and therefore may comprise a nucleic acid sequence encoding rod opsin and arrestin, as described herein. Rod opsin and arrestin may be provided as a separate coding sequences or as s single sequence to be expressed as a fusion protein.

A viral vector has the ability to enter a cell. However, a non-viral vector such as plasmid may be complexed with an agent to facilitate its uptake by a target cell. Such agents include polycationic agents. Alternatively, a delivery system such as a liposome based delivery system may be used.

A vector for use in the present invention is preferably suitable for use in vivo or in vitro, and is preferably suitable for use in a human.

A vector will preferably comprise one or more regulatory sequences to direct expression of the nucleic acid sequence in a target retinal cell. A regulatory sequence may include a promoter operably linked to the nucleic acid sequence, an enhancer, a transcription termination signal, a polyadenylyation sequence (e.g. SV$) late polyA), an origin of replication, inverted terminal repeat sequence, a nucleic acid restriction site, and/or a homologous recombination site. In an embodiment, a vector may comprise a Woodchurch Hepatitis Virus post-translational regulatory element (WRPE) (PMID: 10515449 DOI: 10.1089/10430349950016942). A vector may also include a selectable marker, for example to determine expression of the vector in a growth system (for example a bacterial cell) or in a target retinal cell.

Suitably, a vector is an AAV vector, such as AAV serotype (AAV serotype 1 to AAV serotype 12) or a modified version of any of these serotypes including AAV 4YF and AAV 7m8 vectors, comprising a regulatory sequence to direct expression of a nucleic acid sequence to an inner retinal cell, for example a promotor as described herein, such as Grm6-SV40 for selective targeting of ON-bipolar cells.

Administration: The present invention provides for the administration of a nucleic acid sequence to a cell, suitably to the retina in order to restore photoreceptive ability to the retina. In some embodiments, the composition is administered by injection, preferably an intra-ocular injection, preferably a sub-retinal or intra-vitreal injection.

Where the invention comprises administration of two or more vectors or polypeptides, the method may comprise injecting said vectors/polypeptide into the vitreal cavity, separately, simultaneously or sequentially. In a preferred embodiment, a method of the invention comprises injecting a single dose. Suitably, a method comprises injecting a i) nucleic acid sequence encoding an opsin polypeptide and ii) an arrestin polypeptide into the vitreal cavity of an eye. Preferably, the invention provides a single injectable dose comprising i) a nucleic acid sequence encoding rod opsin; and ii) a nucleic acid encoding an arrestin mutant, for introduction into the vitreal cavity of an eye to provide a photoreceptor function to a cell, for example to restore vision, preferably for treatment of a retinal degenerative condition for example a retinal dystrophy including a rod dystrophy, a rod-cone dystrophy, a cone-rod dystrophy, a cone dystrophy and a macular dystrophy; another forms of retinal or macular degeneration, an ischaemic condition, uveitis and any other disease resulting from loss of photoreceptor ability.

Method of Modulation and Treatment: The methods described herein can be useful for modulating the spatiotemporal activation and deactivation of G-protein signaling. The methods described herein can also be useful for modulating the spatiotemporal activation in optogenetic applications, systems, and technologies. The methods described can achieve spatiotemporal modulation through a mechanism comprising modulating the photoactivity lifetime of photoactive GPCRs in cells and physiological systems. Described herein are methods that can modulate the spatiotemporal activation and deactivation of GPCRs by modulating the intrinsic photoactivity of GPCRs or by modulating photoactivity through GPCR-binding molecules that inhibit and/or quench a photoactive state in cells and physiological systems. The methods described herein can also utilize the spatiotemporal modulation of photoactive GPCRs for applications in cells that do not innately express a photoactive GPCR. The methods described herein can further utilize the spatiotemporal modulation of photoactive GPCRs for therapeutic applications in cells marked by a diseased or abnormal state wherein function of a photoactive opsin is absent or reduced.

The methods described herein can utilize GPCR-binding polypeptides to modulate the spatiotemporal activation of photoactive GPCRs in response to light. A GPCR-binding polypeptide can quench an activated and subsequently modulate photoactive GPCR signaling by modulating the amplitude of a GPCR signal and altering the longevity of a response. Modulation by GPCR-binding polypeptides typically requires the presence of a G-protein specific protease and site-specific GPCR-phosphorylation. The methods described herein can comprise a photoactive GPCR and a GPCR-binding polypeptide capable of binding the GPCR independent of GPCR phosphorylation and modulating GPCR activity. The composition described herein can also utilize mutation within a photoactive GPCR to modulate the spatiotemporal activation of G-protein signaling in response to light. A GPCR mutation can modulate photoactive GPCR signaling by modulating the amplitude of a GPCR signal and altering the longevity of a response. Mutations within a photoactive GPCR can modulate the amplitude of a GPCR signal and altering the longevity of a response through the stabilization of an inactive state or destabilization of an active state. In some cases, the photoactive GPCR is opsin and the GPCR-binding polypeptide is arrestin.

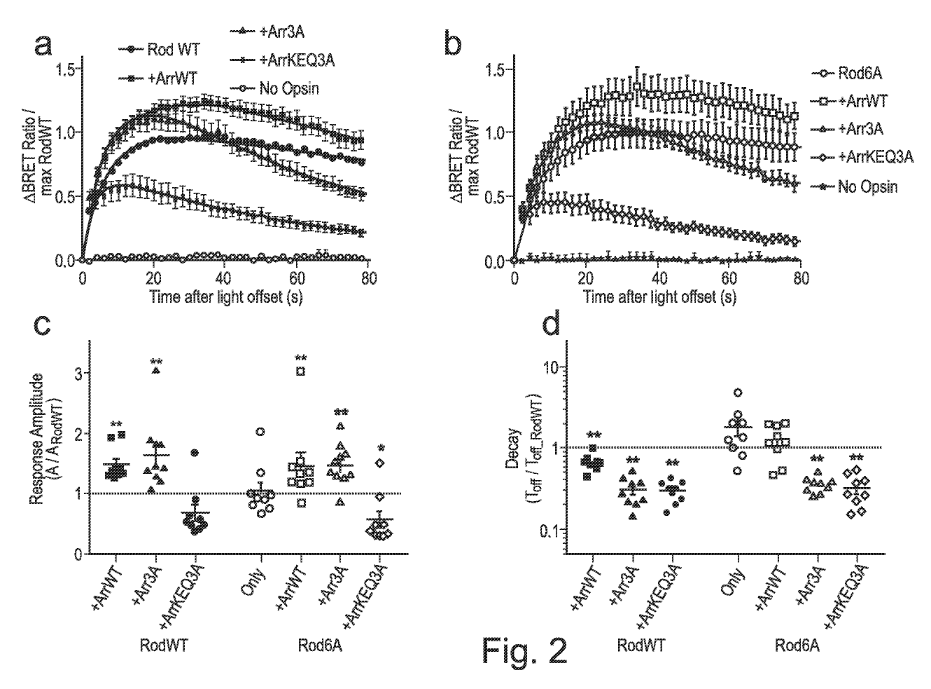

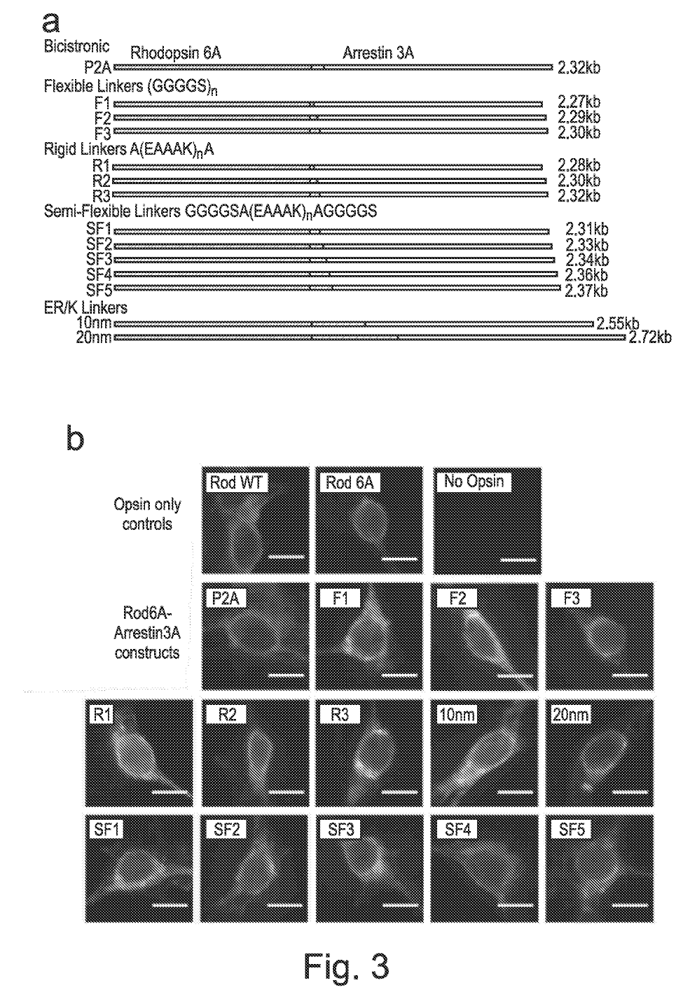

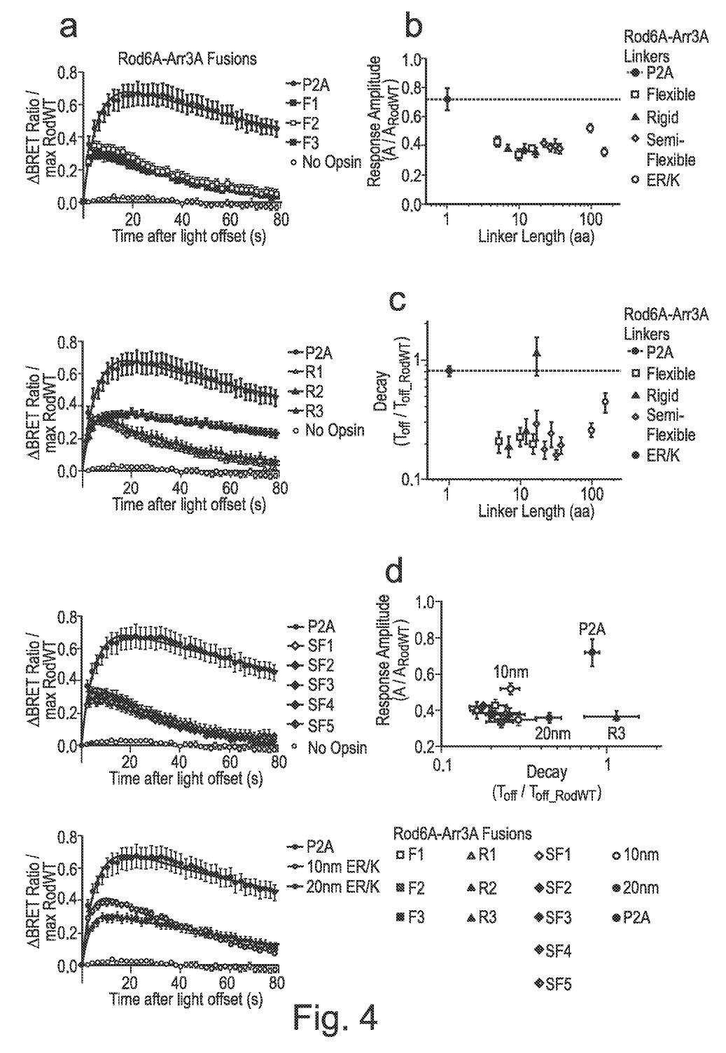

The rod opsin-binding protein arrestin can quench an activated rod opsin molecule and subsequently modulate opsin signaling by modulating the amplitude of a opsin activation signal and altering the longevity of a signaling response. Disclosed herein, in some cases, is a method of increasing deactivation of an opsin, comprising contacting the opsin to a phosphorylation independent arrestin mutant. In some embodiments, the method of increasing a temporal resolution of an opsin light response includes co-transfecting an opsin and a phosphorylation independent arrestin mutant as shown in the example of FIG. 2. In some embodiments, the method of increasing a temporal resolution of an opsin light response includes transfecting a fused protein comprising an opsin and a phosphorylation independent arrestin mutant as shown in the example of FIG. 3.

Disclosed, in some cases, is a method of providing photoreceptor function to a cell lacking photoactivity. Some embodiments relate to a method of increasing deactivation of an opsin polypeptide, comprising administering an effective amount of a composition comprising an opsin polypeptide comprises at least one mutation increasing a rate of Schiff base hydrolysis or a rate of meta-II decay. Some embodiments relate to a method of increasing a temporal resolution of an opsin light response, comprising administering an effective amount of a composition comprising an opsin polypeptide comprises at least one mutation increasing a rate of Schiff base hydrolysis or a rate of meta-II decay.

The opsin polypeptide used in the methods described herein can comprise at least one mutation associated with one or more phosphorylation sites. In some embodiments, the mutation is associated with a C-terminal phosphorylation site. In some embodiments, the opsin polypeptide comprises at least a mutation selected from the group consisting of L59Q, Y74F, E122Q, A132L, A132S, Y136F, I189P, Y227F, Y306F, and a combination thereof. In some embodiments, the mutation results in amino acid substitution, deletion, or addition at the one or more phosphorylation sites of the opsin polypeptide. In some embodiments, the opsin polypeptide comprises a sequence having at least 70%, 80%, 90%, 95%, or 100% sequence identity to SEQ ID NO. 4.

Some embodiments relate to a method of increasing deactivation of an opsin polypeptide, comprising contacting the opsin polypeptide to an arrestin polypeptide having a mutation that increases binding between the arrestin polypeptide and unphosphorylated opsin. Some embodiments relate to a method of increasing a temporal resolution of an opsin light response, comprising administering an effective amount of a composition comprising an arrestin polypeptide having a mutation that increases binding between the arrestin polypeptide and unphosphorylated opsin.

In some embodiments, the arrestin polypeptide comprises a sequence having at least 70%, 80%, 90%, 95%, or 100% sequence identity to SEQ ID NO. 1. In some embodiments, the arrestin polypeptide comprises a sequence having at least 70%, 80%, 90%, 95%, or 100% sequence identity to SEQ ID NO. 2. In some embodiments, the arrestin polypeptide comprises at least a mutation selected from the group consisting of L377A, V378A, F379A, K261Q, E350H, Q332K, or a combination thereof. In some embodiments, the arrestin polypeptide comprises at least a mutation selected from the group consisting of L377A, V378A, F379A, and a combination thereof.

Some embodiments relate to a method of providing photoreceptor function to an inner retinal cell, comprising administering an effective amount of the composition described herein. Some embodiments relate to a method of increasing a temporal resolution of an opsin light response comprising administering a polynucleotide comprising a nucleic acid sequence encoding the composition described herein. Some embodiments relate to a method of treating a retinal degenerative condition in a subject in need thereof, comprising administering an effective amount of the composition described herein.

In some embodiments, the retinal degenerative condition is a retinal dystrophy, a rod dystrophy, a rod-cone dystrophy, a cone-rod dystrophy, a cone dystrophy and a macular dystrophy; other forms of retinal or macular degeneration, an ischaemic condition, uveitis or a condition resulting from loss of photoreceptor ability.

Some embodiments relate to a method for generating photoactivatable cells comprising administering a polynucleotide comprising a nucleic acid sequence encoding the composition described herein. In some embodiments, the polynucleotide further comprises a polynucleotide expression vector. In some embodiments, the polynucleotide further comprises a nucleic acid sequence encoding a GRK1 G-protein coupled receptor kinase.

In some embodiments, the polynucleotide is incorporated into a genome of the cell. In some embodiments, wherein the polynucleotide is incorporated into a genome of the cell. In some embodiments, the polynucleotide is constitutively expressed. In some embodiments, the polynucleotide is transiently expressed. In some embodiments, the cell comprises a neuronal cell. In some embodiments, the cell comprises a neuronal stem cell. In some embodiments, the cell comprises an inner retinal cell. In some embodiments, the inner retinal cell is an ON-bipolar cell, an OFF-bipolar cell, a horizontal cell, a ganglion cell and/or an amacrine cell.

“Extracellular matrix degredation enzyme”: An extracellular matrix degradation protein may be selected from the group consisting of a collagenase, hyaluronan lyase, heparinase I, heparinase II, heparinase III, chondroitin ABC lyase, chondroitin AC lyase, a metalloproteinase, an ADAMTS, a plasmin (serine protease plasmin or its truncated form microplasmin (Ocriplasmin)), neutrophil elastase and cathepsin G, neuraminidase, N-glycanase, O-glycanase, and pronase. A particularly preferred enzyme may be selected from the group consisting of Hyaluronan lyase from Streptomyces hyalurolyticus (EC 4.2.2.1; contained within Genbank accession CP003990); Hyaluronidase from bovine testes (EC 3.2.1.35); chondroitin ABC lyase from Proteus vulgaris (EC 4.2.2.4) and heparinase III from Flavobacterium heparinum (EC 4.2.2.8; Genbank accession L12534, preferably version L12534.1). Enzymes for use in the present invention are available from commercial sources, for example Sigma Aldritch.

By “degrade” or “degradation enzyme” means an enzyme which is capable of breaking down a protein or carbohydrate. A protein can be broken into peptide sequences or amino acids, for example by hydrolysis of the peptide bond. A carbohydrate may be broken down into oligosaccharides or single sugar units. A protein and/or carbohydrate may be fully or partially degraded, meaning that a portion of it may be broken down into smaller fragments, whereas the remainder of the protein and/or carbohydrate may be in its native form. Preferably, a degraded extracellular matrix protein or carbohydrate loses some ability to provide structural and/or biochemical support to a cell, such that a nucleic acid sequence introduced into the vitreous can better access a retinal cell. In particular, a degraded extracellular matrix protein loses some or all its ability to impede movement of a nucleic acid sequence (e.g. gene delivery vector, such as a viral vector), within the vitreous, and into and across the retina. Any loss in extracellular matrix function is sufficiently minimal so that it does not have any significant adverse effect on the eye or vision.

Herein, reference to an extracellular matrix degradation enzyme includes active fragments thereof. An active fragment may be a portion or shorter version of the native enzyme, which retains the ability to function as an extracellular matrix degradation enzyme i.e. it retains the ability to degrade an extracellular matrix protein or carbohydrate, as defined herein. An active fragment may comprise 70%, 75%, 80%, 85%, 90%, 95%, 96%, 97%, 98%, or 99% of the sequence of the native enzyme.

Herein, reference to an enzyme includes one or more enzymes. Thus, the invention provides for the co-administration of a single enzyme or a combination of two or more enzymes. Preferably, where two or more enzymes are provided, they are each selected from the group defined above. Where two or more enzymes are administered, they may be provided in separately, sequentially, or two or more may be provided in combination. Preferably, two enzymes are administered in combination. Where two or more separate doses of enzyme are provided, any one or more of these may be provided in combination with the nucleic acid sequence.

An enzyme for use in the present invention may be derived from any suitable source. The source may be mammalian or non-mammalian. It may be derived from an animal, plant, bacterial, or archeabacterial source. Where mammalian, it is preferred that it is a human enzyme. It may be isolated or purified from such a source. It may be produced as a recombinant protein. Alternatively, it may be synthetically produced.

The nucleic acid and amino acid sequences of enzymes for use in the present invention are known in the art.

Herein, enzymes include fragments and derivatives of native enzymes. Preferably a fragment or derivative shares at least 70%, 75%, 80%, 85% or 90%, at least 91, 92, 93, 94, 95, 96, 97, 98, or at least 99% sequence identity with a native enzyme, over a length of 50%, 60%, 70%, 80%, 90%, or at least 95% of the length of a native enzyme.

An enzyme for use in the present invention may be provided in dry form, which includes either dehydrated or lyophilised forms. Typically, an enzyme will be provided in lyophilised form. Alternatively, an enzyme may be provided as an aqueous solution, for example pre-dissolved in water at a predetermined concentration and volume. For administration, an aqueous form is preferred, although it is envisaged that a product or kit of the invention may suit the provision of a dried form of the enzyme, optionally with instructions for dissolving. Thus, a method of the invention may comprise using a dried enzyme to produce an enzyme solution. Preferably, this is achieved by dissolving or reconstituting the enzyme in an aqueous or non-aqueous solvent. Suitable solvents are those which are non-toxic, and suitable for use with humans or animals. Preferably, a suitable solvent is sterile. An example of a suitable solvent is sterile phosphate buffered saline. Methods for dissolving dried proteins are known in the art.

Co-administration of an enzyme includes separate, sequential or combined administration during the same therapy). Administration of an enzyme may be limited to the vitreous humour. An enzyme does not need to be targeted to a retinal cell. In aspects of the invention comprising administration of an extracellular matrix degradation enzyme, an enzyme may be provided separately or in combination with a vector, polypeptide or composition of the invention i.e. as a single composition. Where provided separately, they may be provided in the same excipient or in different excipients. In such an embodiment, the may be held separately, for example in separate microcapsules. Thus, in a preferred embodiment, the enzyme may be provided as a separate injectable liquid, suitably in a separate container, such as a capsule or syringe.

FIG. 1. Using BRET assay of G protein activation to assess kinetics of opsin-driven light responses in presence of rod photoreceptor deactivation mechanisms: FIG. 1a) Ratiometric BRET assay of G protein activation. Dissociation of the G protein heterotrimer is detected when BRET occurs between free Gβγ-dimer tagged with yellow fluorescent protein (Venus) and membrane-localised GRK3-fragment fused to Nanoluciferase (which continuously emits light at 470 nm), leading to emission of 535 nm light. As G protein heterotrimer reassociates, the binding site for GRK3 fragment is blocked by Gα subunit and emission of 535 nm fluorescent light decreases. BRET Ratio is calculated as light emitted at 535 nm/light emitted at 470 nm; FIG. 1b) Temporal resolution of rod-opsin driven light responses with BRET assay of G protein activation (BRET Go - Black) is faster than for secondary messenger Glosensor Gso assay (Glo + Gso- Grey); FIGS. 1c-1d) Time course of BRET response to 1 s 470 nm light (14.1 log photons) normalised to pre-flash baseline (=0) and maximum response from wildtype Rod opsin positive control (= 1). Responses show c) wildtype Rod opsin (Rod WT - filled makers) or d) phosphonull Rod opsin 6A mutant (Rod 6A - unfilled markers) in presence of either G protein receptor kinase 1 (GRK1) and/or visual arrestin (Arr); FIG. 1e) Baseline normalised BRET responses are modelled using a simple dual exponential model (left panel) consisting of a scaling factor (A), a one-phase exponential association curve (Rₒₙ) and one-phase exponential decay curve (R_{off}). The three parameters that define this model, A, Tₒₙ and T_{off}, are adjusted to fit model to data using non-linear regression (right panel). The Best fit parameter A is used as a measure of response amplitude and T_{off} is used as a measure of response decay rate. FIG. 1f) Relative response amplitude, measured as best fit scaling factor (A) for each condition divided by best fit scaling factor for rod opsin positive control (A_{Rod_WT}). FIG. 1g) Relative response decay, measured as best fit T_{off} (s) for each condition divided by best fit T_{off} for rod opsin positive control (T_{off_}_{RodWT}).

Glosensor and BRET data shown in b,e) are mean ± standard error of mean for n = 3 replicates. BRET data shown in c,d,f-g) are mean ± standard error of mean for n = 8-9 replicates from 3 separate transfections. For statistical analysis, a two-tailed Wilcoxon signed ranks test was used to compare each condition relative to Rod control (Theoretical median = 1). ** p < 0.01; * p < 0.05; not significant where no asterisk is displayed. Grey asterisk = significant for uncorrected alpha (0.05), Black asterisk = significant for Sidak corrected alpha (0.007 for A, 0.009 for T_{off}).

FIG. 2. Phosphorylation-independent arrestin mutants shorten lifetime of rod opsin activity without suppressive effects of GRK1: FIGS. 2a-2b) Time course of BRET light responses for a) wildtype Rod opsin (RodWT) and b) phosphonull Rod opsin 6A mutant (Rod6A) when co-transfected with either wildtype visual arrestin (ArrWT) or arrestin mutants with intermediate (Arr3A) or strong affinity (ArrKEQ3A) for unphosphorylated active rod opsin. Responses are to 1 s 470 nm light (14.1 log photons). Data are normalised to pre-flash baseline (=0) and maximum response of Rod WT (=1). FIG. 2c) Response decay (measured as fold change in best fit T_{off}, s) of both RodWT and Rod6A is decreased when arrestin mutants Arr3A and ArrKEQ3A are added. FIG. 2d) Response amplitude (measured as fold change in best fit scaling factor A) of both RodWT and Rod6A is partially increased in presence of ArrWT and Arr3A, but not ArrKEQ3A.

Data are mean ± standard error of mean of n = 9 replicates from 3 separate transfections.

In FIGS. 2c-2d) a two-tailed Wilcoxon signed ranks test was used to compare each condition relative to Rod control (Theoretical median = 1). * p < 0.05; ** p < 0.01; not significant where no asterisk is displayed. Grey asterisk = significant for uncorrected alpha (0.05), Black asterisk = significant for Sidak corrected alpha (0.007 for A and T_{off}).

FIG. 3. Rod opsin-Arrestin fusions have normal subcellular localisation. FIG. 3a) Diagram of Rod opsin-Arrestin 3A bicistronic and fusion constructs. Linkers with different biophysical properties and lengths were used to produce 13 fusion constructs. Length of rod opsin, arrestin and linkers are to scale, with construct size in kilo base pairs shown on right. FIG. 3b) Heterologous expression of wildtype Rod opsin (Rod WT) only, phosphonull Rod opsin 6A only, Rod opsin6A co-expressed with Arrestin 3A (P2A), or Rod opsin 6A tethered to Arrestin 3A by linker in Hek293T cells labelled with anti-rhodopsin 4D2 Scale bar 10 µm.

FIG. 4. Rod opsin 6A - Arrestin3A fusions have improved temporal resolution but reduced response amplitude compared to co-expression: FIG. 4a) Time course of BRET light responses for co-expression or fusions of phosphonull rod opsin mutant (Rod6A) and intermediate affinity arrestin mutant 3A (Arr3A). Responses are to 1 s 470 nm light (14.1 log photons). Data are normalised to pre-flash baseline (=0) and maximum response of rod opsin positive control (=1); FIG. 4b) Response amplitude (measured as fold change in best fit scaling factor A from Rod opsin positive control) is decreased for fusion constructs compared to co-expression with bicistronic P2A vector (dashed line), with no obvious relationship of linker length or composition with amplitude. FIG. 4c) Response decay (measured as fold change in best fit T_{off} ,s from Rod opsin positive control) is faster for most fusion constructs compared to co-expression with P2A (dashed line). Linker R3 was notably very slow to deactivate. FIG. 4d) Comparing response amplitude and decay (measured as scaling factor A and T_{off} , respectively) of Rod6A-Arr3A fusions shows most linker lengths and compositions result in similar response properties. Of all fusion constructs, the ERK 10 nm linker provided the largest response amplitude while maintaining fast response decay.

Data shown are mean ± standard error of mean of n = 10-13 replicates for P2A and fusions and n = 20 for RodWT from 4 separate transfections.

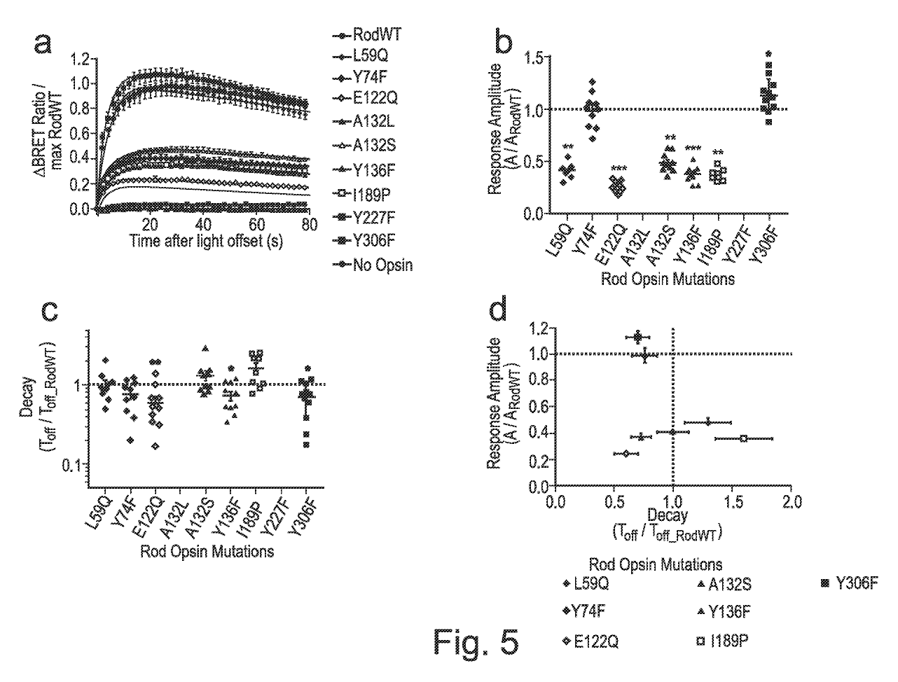

FIG. 5. Rod opsin mutants with increased meta-II decay have variable response amplitude and G protein deactivation: FIG. 5a) Time course of BRET light responses for rod opsin wildtype (RodWT) and mutants reported to have faster meta-II decay. Responses are to 1 s 470 nm light (14.1 log photons). Data are normalised to pre-flash baseline (=0) and maximum response of rod opsin positive control (=1). FIG. 5b) Response amplitude (measured as fold change in best fit scaling factor A from Rod opsin positive control) is generally smaller for meta-II decay mutants, compared to RodWT, although Y74F and Y306F have comparable and larger amplitudes respectively. FIG. 5c) Response decay (measured as fold change in best fit T_{off} ,s from Rod opsin positive control) varies across rod opsin meta-II decay mutants, with E122Q, Y136F and Y306F showing faster responses, and L59Q and I189P showing trend towards slower responses. FIG. 5d) Comparing relative response amplitude and decay of Rod opsin mutants shows the two parameters are not strongly correlated.

Data shown are mean ± standard error of mean of n = 9-11 replicates from 3 separate transfections. In FIGS. 5c-5d) a two-tailed Wilcoxon signed ranks test was used to compare each condition relative to Rod control (Theoretical median = 1). * p < 0.05, ** p < 0.01; *** p < 0.001, not significant where no asterisk is displayed. Grey asterisk = significant for uncorrected alpha (0.05), Black asterisk = significant for Sidak corrected alpha (0.007 for A and T_{off}).

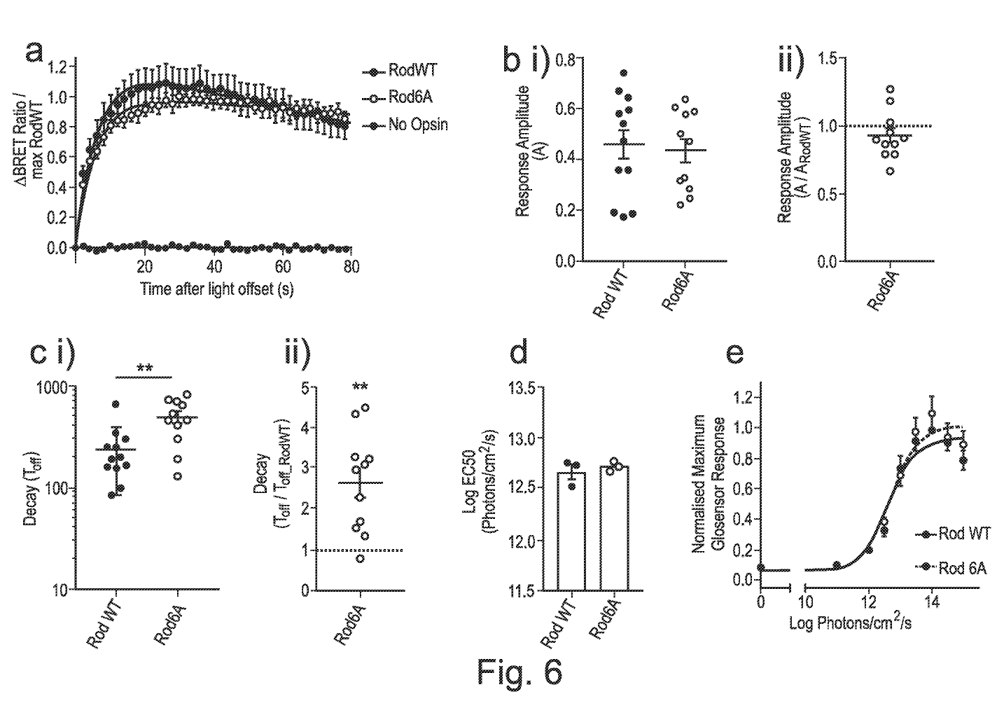

FIGS. 6a-6e. Comparing response properties of rod opsin wildtype (Rod WT) and phosphonull rod opsin (Rod6A): FIG. 6a) Time course of BRET response to 1 s 470 nm light (14.1 log photons). Data are normalised to pre-flash baseline (=0) and maximum response of rod opsin (=1). FIG. 6b) Response amplitude, measured as best fit scaling factor (A) normalised to RodWT, is similar between Rod6A and RodWT, two-tailed Mann-Whitney U-test, p = 0.694. FIG. 6c) Response decay measured as best fit Toff (s) is slower in Rod6A compared to RodWT, two-tailed Mann-Whitney U-test, p = 0.007. FIG. 6d) Sensitivity (measured as Log EC50, log photons/cm2/s using Glosensor Gso assay) is comparable between Rod6A and RodWT. FIG. 6e) Irradiance response curves (IRC) fit to Rod WT or Rod 6A-driven responses to increasing intensities of 470 nm light (11-15 log photons) measured using Glosensor Gso assay.

Data are mean ± standard error of mean. BRET data are mean of n = 11-12 replicates from 4 separate transfections. Glo Gso data are mean of 3 independent experiments. ** p > 0.01, p > 0.05 where no asterisk is displayed.

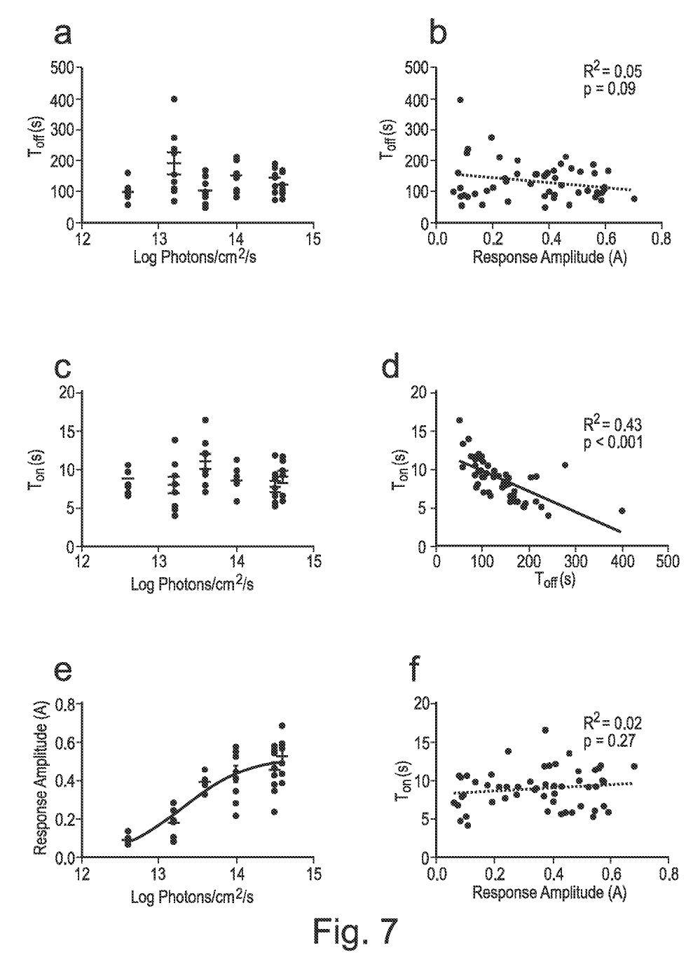

FIG. 7. Modelling Rod opsin-driven BRET responses to different light intensities: Rod opsin-driven to 1 s flashes of varying intensities (12.3 - 14.6 log photons/cm2/s) of 470 nm light were measured using BRET assay and fit with simple 3 parameter model (A, Ton and Toff) using non-linear regression. The same opsin activated to different levels should have similar rates of response onset and response decay, FIG. 7a) The response decay rate (measured as Toff) is consistent across responses to different intensity flashes (ie: different levels of G protein activity). FIG. 7b) Response decay does not significantly correlate with response amplitude (Pearson’s product-moment correlation, r = -0.23, R2 = 0.05, p = 0.097). FIG. 7c) The response onset (measured as Ton) is also consistent across intensities and FIG. 7d) does not significantly correlate with response amplitude (Pearson’s product-moment correlation, r = 0.15, R2 = 0.02, p = 0.275). FIG. 7e) Response amplitude is irradiance-dependent, consistent with increasing levels of G protein activity at higher intensities. These data can be fit with an irradiance response curve (IRC) with log EC50 = 13.31, R2 = 0.74. FIG. 7f) Two parameters, Ton and Toff, are significantly negatively correlated with longer lifetime responses showing shorter response onset (Pearson’s product-moment correlation, r = -0.66, R2 = 0.43, p < 0.0001).

For FIGS. 7a, 7c, 7e) a model comparison (F-test) was performed to determine if data were better fit by a horizontal line (null hypothesis) or a sigmoid irradiance response curve (IRC, alternative hypothesis) using non-linear regression. For data where null hypothesis was rejected, such as response amplitude vs intensity, (F(2,50) =7086, p < 0.0001, the best fit IRC is displayed. For response decay and response onset vs intensity, null hypothesis was not rejected (p > 0.05). Error bars show standard error of the mean.

For FIGS. 7b,7d, 7f) the best-fit linear trendline is displayed (solid line for significant correlations, dashed line for non-significant correlations). Data are from 3 independent experiments (3 technical replicates each).

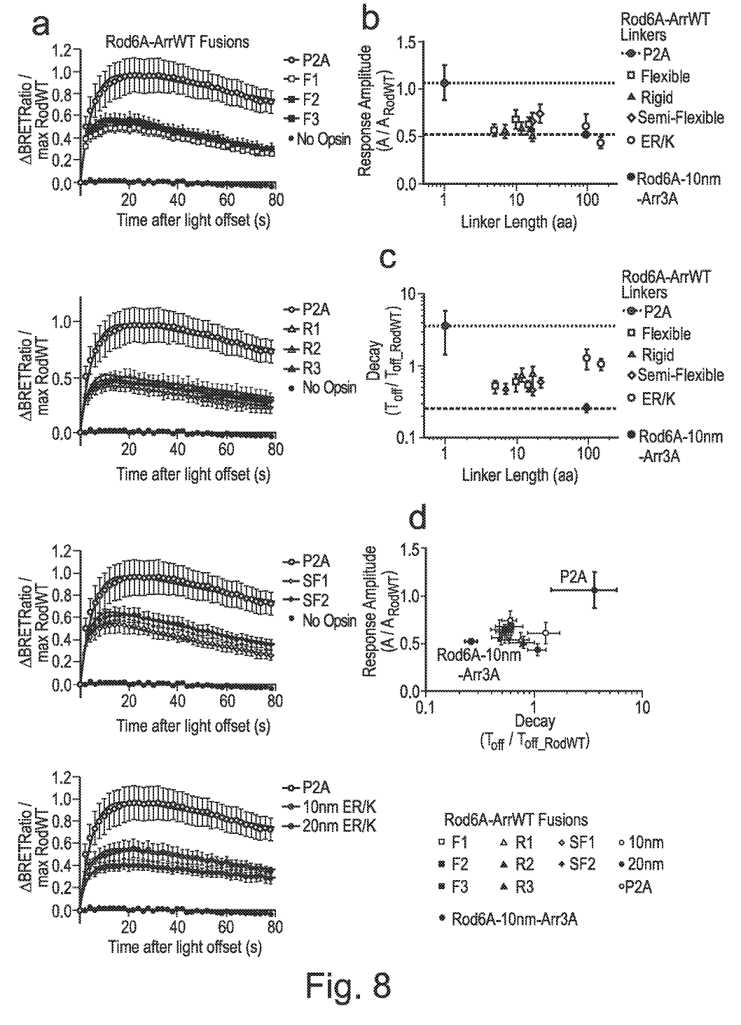

FIG. 8. Rod opsin 6A - ArrestinWT fusions have reduced response amplitude but faster temporal resolution compared to co-expression. FIG. 8a) Time course of BRET light responses for co-expression or fusions of phosphonull rod opsin mutant (Rod6A) and wildtype arrestin (ArrWT). Responses are to 1 s 470 nm light (14.1 log photons). Data are normalised to pre-flash baseline (=0) and maximum response of rod opsin positive control (=1). FIG. 8b) Response amplitude (measured as fold change in best fit scaling factor A from Rod opsin positive control) is decreased for fusion constructs compared to co-expression with bicistronic P2A vector (dashed line), with no obvious relationship of linker length or composition with amplitude. Response amplitude for most fusions is comparable to best-performing Rod6A-Arr3A fusion with 10 nm linker (dotted line). FIG. 8c) Response decay (measured as fold change in best fit Toff ,s from Rod opsin positive control) is faster for all fusion constructs compared to co-expression with P2A (dashed line). Response decay for R6-ArrWT fusions is slower than for best performing Rod6A-10 nm-Arr3A fusion (dotted line). FIG. 8d) Comparing response amplitude and decay (measured as scaling factor A and Toff, respectively) of Rod6A-ArrWT fusions show all have slower decay and similar response amplitude to best-performing Rod6A-Arr3A fusion, Rod6A-10nm-Arr3A.

Data shown are mean ± standard deviation of n = 12-13 replicates for Rod6A-ArrWT fusions and n = 17 for RodWT from 4 separate transfections.

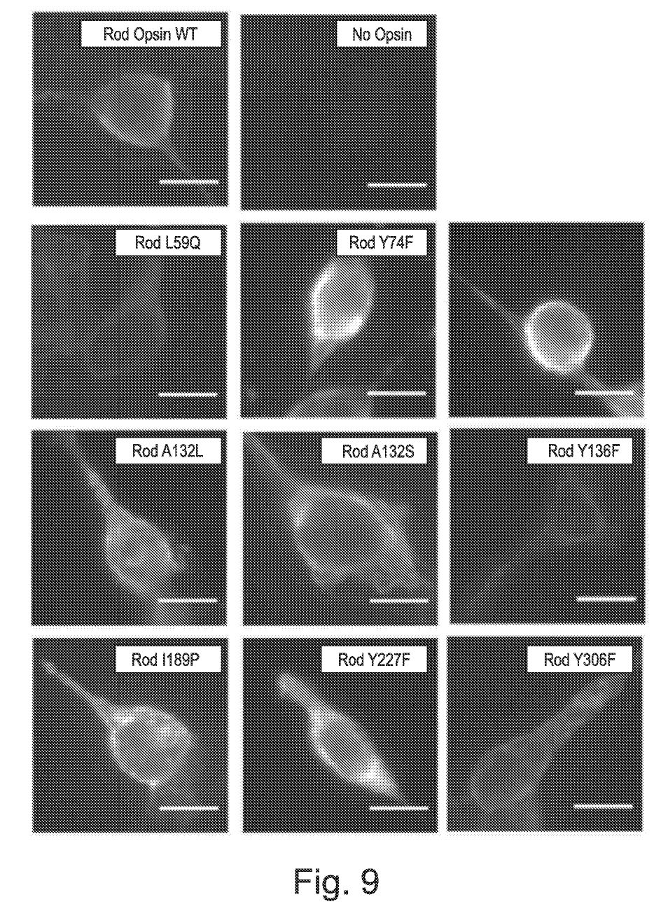

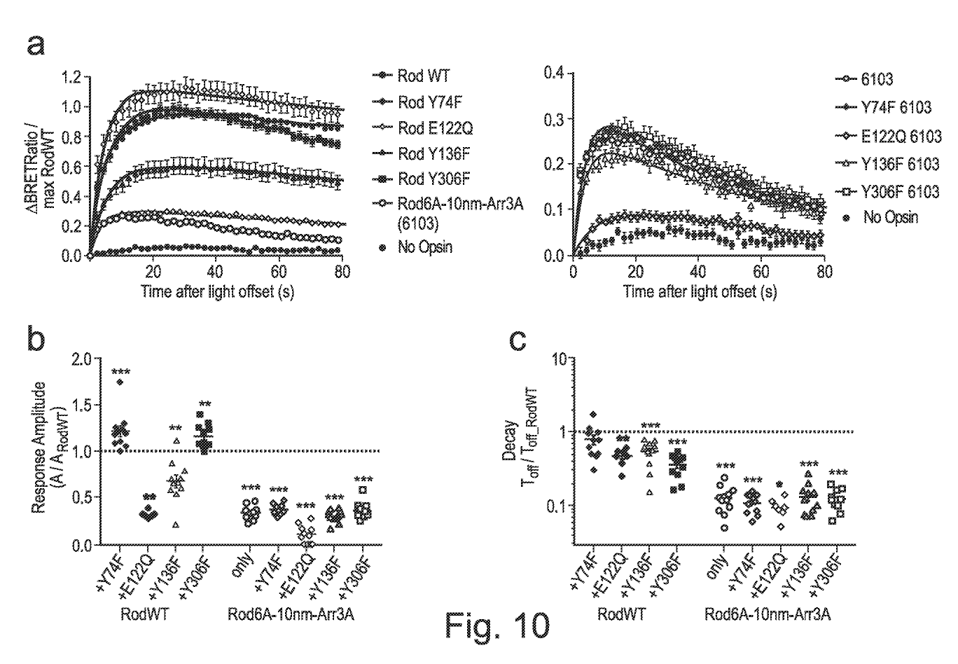

FIG. 9. Rod opsin meta-II decay mutants have normal subcellular localisation. Heterologous expression of wildtype Rod opsin (Rod WT) or rod opsin meta-II decay mutants in Hek293T cells labelled with anti-rhodopsin 1D4 antibody. Scale bar 10 µm. In FIG. 10, no detectable advantage was observed in combining rod opsin meta-II decay mutants with phosphorylation-independent arrestin mutants: FIG. 10a) Time course of BRET light responses to 1 s 470 nm light (14.1 log photons). Data are normalised to pre-flash baseline (=0) and maximum response of rod opsin control (=1). Meta-II decay mutants applied to Rod WT are shown as filled circles, while meta-II decay mutants applied to Rod6A-10nm-Arr3A fusion (6103) are shown as unfilled circles. FIG. 10b) Response amplitude (measured as fold change in best fit scaling factor A from Rod opsin positive control) is significantly attenuated for all Rod6A-10 nm-Arr3A fusion with meta-II decay mutants, and is comparable with relative amplitude of Rod6A-10 nm-Arr3A without meta-II decay mutation. We were unable to accurately fit model to 4/11 replicates for E122Q R6-10 nm-Arr3A, missing values are given response amplitude value of zero to avoid biasing average towards replicates with larger amplitudes. FIG. 10c) Response decay (measured as fold change in best fit T_{off} ,s from Rod opsin positive control) is also comparable for all Rod6A-10 nm-Arr3A fusions with meta-II decay mutants. Relative Toff is shown for E122Q Rod6A-10 nm-Arr3A responses that could be fit using 3 parameter model (7/11 replicates).

Data shown are mean ± standard error of mean of n = 8-12 replicates from 3 separate transfections. In b-c) a two-tailed Wilcoxon signed ranks test was used to compare each condition relative to Rod control (Theoretical median = 1). * p < 0.05, ** p < 0.01; *** p < 0.001, not significant where no asterisk is displayed. Grey asterisk = significant for uncorrected alpha (0.05), Black asterisk = significant for Sidak corrected alpha (0.006 for A and T_{off}).

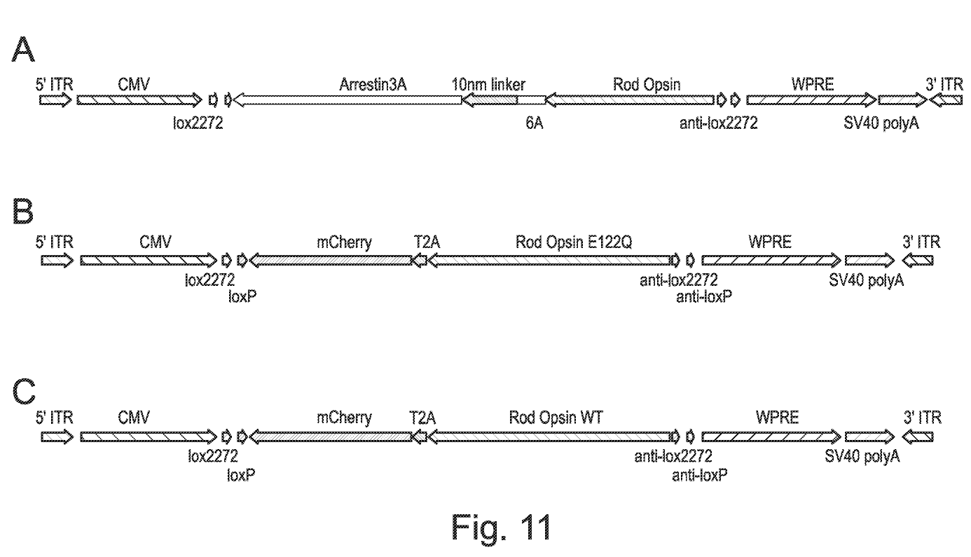

FIG. 11 shows a schematic of optogenetic viral transgenes. AAV2 Quad-YF A) Rod6A-10nm-Arr3A, B) Rod Opsin E122Q and C) Rod Opsin Wildtype. B) and C) are co-expressed with fluorescent mCherry reporter via T2A peptide.

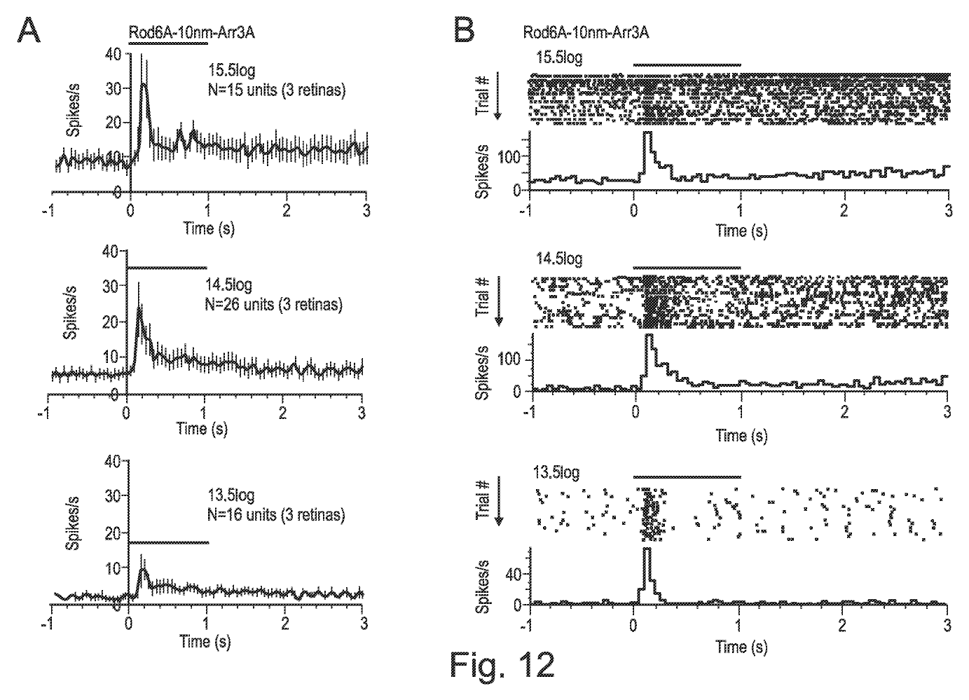

FIG. 12 shows light responses from retinas transduced with Rod6A-10nm-Arr3A AAV. A) Average firing rate (spikes/s) across light-responsive units. B) Representative responses from individual units. All representative traces show perievent rasters (first trial at top) and associated perievent firing rate histograms (Bin size = 50 ms). Timing of light stimuli shown by horizontal black bar. Error bars show standard error of mean. Stimulus intensity is given in log rod effective photons/cm²/s.

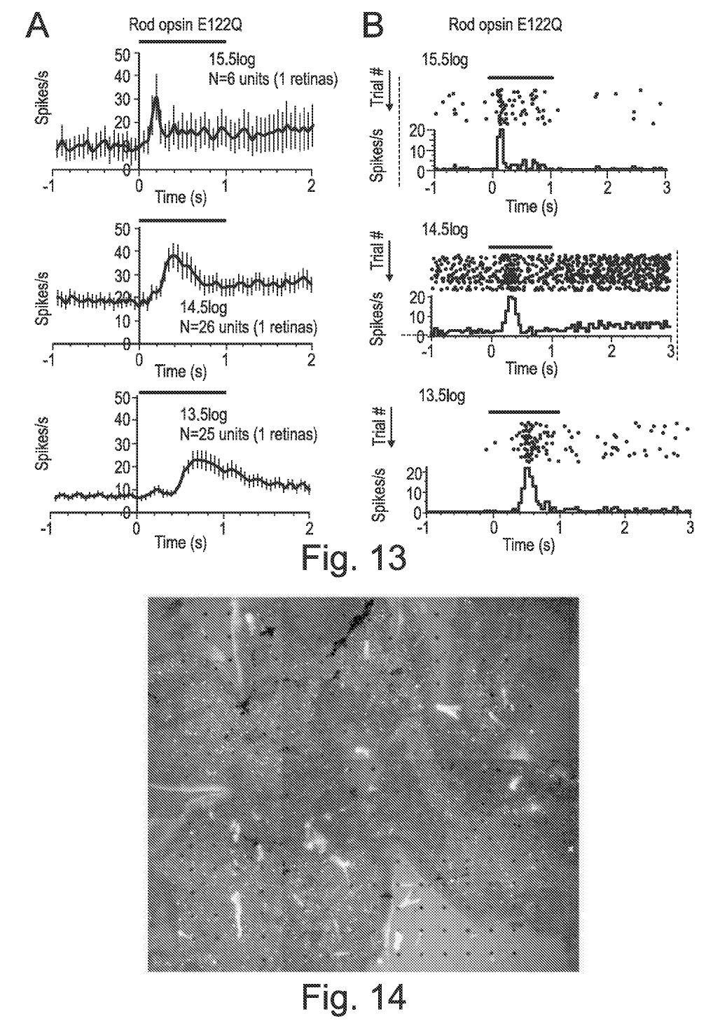

FIG. 13. Light responses from retinas transduced with Rod opsin E122Q AAV. A) Mean firing (spikes/s) across light-responsive units. B) Representative responses from different individual units. .All representative traces show perievent rasters (first trial at top) and associated perievent firing rate histograms (Bin size = 50 ms). Timing of light stimuli shown by horizontal black bar. Error bars show standard error of mean. Stimulus intensity is given in log rod effective photons/cm²/s.

FIG. 14. Expression of mCherry report in retina transduced with Rod opsin E122Q-P2A-mCherry AAV. Retina is displayed bipolar-cell side up - small bright dots represent individually transduced bipolar cells.

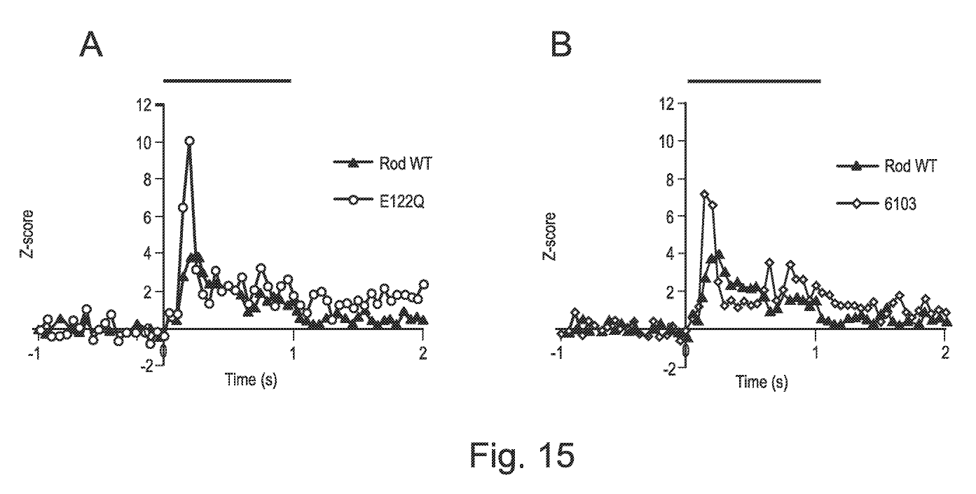

FIG. 15. Rod opsin variants show larger and less sustained responses to light compared to native rod opsin. Changes in firing (expressed as standard deviations from baseline mean - z-score) at electrode sites showing a light response are shown for retinas treated with virus driving Rod opsin E122Q (A, n = 6 units, 1 retina) and Rod6A-10 nm-Arr3 (B, 6103, n = 15 units, 3 retinas), with the response in native rod opsin treated retinas (n = 21, 4 retinas) shown in black on each plot for comparison. Note that in all cases, there is a more abrupt and shor.ter lasting light response in the variants optimised for temporal resolution than in the wild type control. Timing of light stimuli shown by horizontal black bar, data show mean firing. White light stimulus is 15 log rod effective photons/cm²/s

### EXAMPLES

### Example 1

A live cell assay of G protein activation to measure kinetics of opsin signaling. The aim was to improve the temporal resolution of the rod opsin light response under heterologous expression. To accomplish this, we required a high-throughput approach to assess the lifetime of rod opsin activity (how fast it deactivates) that was suitable for screening multiple potential interventions in a live cell environment. We chose to apply a bioluminescence resonance energy transfer (BRET)-based reporter of G protein activation to HEK293 cells expressing rod opsin. In this assay, the dissociation of the G protein heterotrimer is detected by using a fluorescent Venus-tagged Gβγ dimer (sVβγ) with a nanoluciferase-tagged GRK3 fragment (nLuc-GRK3, FIG. 1a). The nLuc-GRK3 fragment has high affinity for free Gβγ, resulting in BRET detected as an increase in the ratio of light emitted by Venus to that emitted by nanoluciferase. Exogenous expression of a Gα subunit renders this assay specific to a single G protein signalling pathway. Rod opsin can activate G-proteins of the Gαi/o/t class in HEK293 cells with good efficiency [Ballister et al, 2018, BMC Biology 16, 10]. In this case, we focused on Gαo, as this is a widely expressed G alpha subunit in the central nervous system and is the G alpha subunit expressed in retinal bipolar cells targeted by optogenetic therapies for vision restoration.

A flash of light drove an increase in BRET in HEK293 cells co-expressing rod opsin with the components of this reporter system (FIG. 1b). Rod-opsin driven light responses have previously been detected in live cells using Glosensor, a luminescent reporter for the second messenger cAMP that is impacted by Gαi/o/t pathways [Bailes et al, 2012, PLoS ONE 7, e30774; 2013, Proc Biol Sci 280]. In a side-by-side comparison, we found the BRET signal both rose and fell much earlier than was the case for the Glosensor response (FIG. 1b), consistent with its ability to report an earlier stage in the phototransduction cascade (G-protein activation) and confirming its superiority for exploring the timeframe of opsin activation.

### Example 2

Quenching rod opsin responses using visual arrestin was tested. We first attempted to improve the temporal resolution of rod opsin during heterologous expression by introducing the components for rod opsin deactivation that are normally present in rod photoreceptors. Co-expression of visual arrestin (Arr) with rod opsin did not itself have a dramatic effect on the rod opsin light response (FIG. 1c). However, the additional inclusion of rod opsin’s native G-protein coupled receptor kinase (GRK1) with Arr resulted in a large change in the amplitude and longevity of the response to a light flash (FIG. 1c). The reduction in amplitude appeared to be largely an arrestin-independent effect as it was also observed when GRK1 was applied without Arr expression (FIG. 1c). To confirm that these effects on the BRET light response reflected introduction of the two-stage arrestin-dependent quenching of rod opsin signalling, we repeated the experiments with rod opsin mutated to remove C-terminal phosphorylation sites (Rod6A). This mutated opsin should not be phosphorylated by GRK1 and must therefore be unaffected by GRK1 and Arr expression. This did indeed appear to be the case, as the major effects of GRK1 and GRK1+Arr expression were lacking in the Rod6A driven light response (FIG. 1d). We observed no differences in the response characteristics of wildtype Rod opsin (RodWT) and Rod6A mutant, apart for slower response decay in Rod6A (FIG. 6).

To facilitate quantitative analysis of these data, we parameterised the BRET response profiles using a simple 3-parameter model (FIG. 1e) which we fit using non-linear regression. This model consisted of an exponential association curve for the response onset (Ron); an exponential decay curve for the response decay (Roff); and a scaling factor (a measure of response amplitude, A). The three parameters that define the components of this model are Ton, Toff and A. Ton is defined by the rate of accumulation of the BRET signal and, as opsin activation by light is effectively instantaneous will be primarily defined by the latency of the BRET assay response. Toff is defined by the rate of decay of the BRET signal and a priori is expected to be influenced both by the rate of opsin deactivation and rate at which BRET signal recovers to baseline in the absence of further G-protein activation. A reflects the peak amplitude of the BRET response. To meet our objective of enhancing temporal resolution we aimed to identify interventions that minimized the life time of photoactivated opsin (reflected in reduced Toff), without impacting peak signal amplitude (as an assay of opsin G-protein signalling efficiency). An important consideration then is whether changes in response amplitude themselves induce alterations in Toff in this assay. We tested this possibility by using variations in flash intensity to produce a range of response amplitudes and found that indeed A and Toff, were not significantly correlated.

Applying this approach confirmed that response amplitude (A) of RodWT was significantly reduced by co-expression with either GRK1 (Wilcoxon signed rank test compared to RodWT, p = 0.004) or GRK1+Arr (p = 0.008, FIG. 1f), to ~25% of RodWT amplitude. According to this analysis, there was also a small increase in response amplitude associated with adding arrestin alone to Rod WT (p = 0.012), from A of 0.57 (SEM = 0.04) to 0.71 (SEM = 0.05) respectively. In comparison, response amplitude of phosphonull Rod6A remained similar to RodWT (p = 0.301) in presence of either Arr (p = 0.496) or GRK1+Arr (p =0.164), with a small reduction in presence of GRK1 only (p = 0.008) to A of 0.42 (SEM = 0.04).

A small decrease in decay (T_{off}) of Rod WT response was found when arrestin was added (p = 0.129), to 90% of RodWT level, consistent with low affinity of arrestin for unphosphorylated rod opsin (FIG. 1g). The decay rate was 2.5x faster when GRK1 + Arr were added to RodWT (p = 0.008), from T_{off} of 166.16 (SEM = 10.06) to 65.57 (SEM = 11.18) consistent with successful restoration of rod opsin deactivation mechanisms. Interestingly, the Rod+GRK1 light response could be fit using a single exponential association function, indicating that the rate of decay was sufficiently slow to be essentially undetectable under these conditions Response decay of Rod6A remained slower than RodWT when Arr (p = 0.004) or GRK1 (p = 0.008) were added. T_{off} was comparable with RodWT when GRK1 only was co-expressed with Rod6A (p = 0.820), as anticipated given that opsin deactivation by arrestin is phosphorylation-dependent (Vishnivetskiy et al., 2007).

### Example 3

Phosphorylation-independent mutants of arrestin was tested. A remarkable outcome of these first set of experiments was the substantial reduction in response amplitude caused by addition of GRK1 (FIGS. 1c & 1f). This effect appeared to be largely attributable to opsin phosphorylation, as it was absent in Rod6A, but independent of arrestin, as it did not require Arr expression. Excluding GRK1 therefore seems preferable for optimising response amplitude. Conversely, however, arrestin requires GRK1-driven phosphorylation to reduce response lifetime. A potential solution to this conundrum is to use phosphorylation-independent arrestin mutants. These arrestin mutants have their “phosphorylation sensor” removed, increasing their affinity for photoactivated but unphosphorylated opsin. We reasoned that if these were able to improve temporal resolution of rod opsin, they would remove requirement for GRK1 phosphorylation and potentially allow larger amplitude responses.

We tested Rod opsin co-transfected with wildtype arrestin (ArrWT) or one of the arrestin mutants, Arr3A and ArrKEQ3A, which have intermediate and high affinity for unphosphorylated rod opsin, respectively [Vishnivestkiy et al, 2007, J. Biol. Chem. 282, 32075-32083] (FIG. 2a). The high affinity arrestin KEQ3A mutant reportedly has similar affinity for R* as wildtype arrestin has for phosphorylated active rod opsin (P-R*). We also tested the arrestin mutants with phosphonull Rod6A to confirm any effects were truly phosphorylation-independent (FIG. 2b).

Both phospho-independent arrestins significantly shortened the lifetime of RodWT (FIG. 2c; mean±SEM Toff = 240.64± 24.13 for RodWT, 69.07±8.03 for +Arr3A, and 68.07±8.03 for RodWT+ArrKEQ3A; Wilcoxon Signed Ranks Test, p = 0.004 for both Arr3A and ArrKEQ3A compared to RodWT). As previously, response decay of RodWT, and to a lesser extent Rod6A, was marginally decreased when co-transfected with ArrWT (mean±SEM Toff = 160.19, ± 21.2 for RodWT, 299.32±53.7 for Rod6A). It appears the higher affinity for R* of the arrestin mutants contributes to faster deactivation, with T_{off}for Rod6A + Arr3A or ArrKEQ3A demonstrating response decay 2.87x and 3.23x shorter than RodWT.

A notable difference between the two arrestin mutants was their impact on response amplitude (FIG. 2d), with a trend for ArrKEQ3A to suppress RodWT and Rod6A (p = 0.128 for RodWT, p = 0.012 for Rod6A) to 68% and 56% of RodWT level respectively, while Arr3A increased response amplitude of RodWT (p = 0.004) and Rod6A (p = 0.008), to 162% and 146% of wildtype control amplitude respectively, comparable to the effect of ArrWT (p = 0.004 for Rod WT, p = 0.008 Rod6A).

### Example 4

Opsin-Arrestin fusions was tested for improving efficiency of opsin deactivation. The combination of Rod6A and Arr3A improved temporal resolution of rod opsin, without affecting response amplitude. We next explored whether physically tethering these two proteins to create fusions may allow even more efficient deactivation. To this end, we designed a series of fusion constructs with a variety of linkers (FIG. 3a), to determine which allowed optimal interaction between Rod6A and Arr3A. These include flexible glycine-serine linkers with a high degree of rotational freedom; rigid alpha-helix forming linkers, which limit interaction of the two proteins; semi-flexible linkers, consisting of a rigid linker with flexible ends, and a ER/K linker, which possess alternating charge, making them unlikely to interact with protein domains at either end. We also varied the length of the different linkers, which when increased will decrease frequency of interaction.

Each Rod6A-Arr3A fusion was compared with co-expression of the two proteins without physical association. To ensure a 1:1 stoichiometric ratio we switched from simply co-transfecting expression vectors for the two components used to generate data in FIG. 2 to employing a single bicistronic vector (Rod6A-P2A-Arr3A) using the self-cleaving P2A sequence to produce the two proteins from a single peptide [Kim et al, 2011, PLoS ONE 6, e18556]. Immunostaining of the fusion constructs (FIG. 3b) revealed all fusions were correctly localised to the plasma membrane and express well.

All Rod6A-Arr3A fusion constructs retained the ability to drive light responses (FIG. 4a), albeit with response amplitude reduced by 1.7x to 2.4x compared to co-expression of the two proteins (FIG. 4b). Turning to response lifetime, a first observation was that introducing Arr3A using the bicistronic vector had a smaller impact on Toff than previously observed in the co-transfection studies (FIG. 2). The origin of this is unclear, but one explanation is that the enforced 1:1 stoichiometry had a detrimental impact on this effect. Tagging did enhance the reduction in T_{off}, with response lifetimes 1.9x to 5.5x shorter shown by all fusions compared to co-expression of Rod6A and Arr3A. (FIG. 4c).

Comparison of response parameters across the various fusion constructs revealed firstly that there was no strong relationship between response amplitude and T_{off} (FIG. 4d). The implication that these parameters are at least partially dissociable in this dataset suggests that our approach is suitable for identifying the fusion that provides the best combination of a light response with high signalling efficiency (large A) and short lifetime (low T_{off}). Of all fusions, Rod6A-10nm-Arr3A offered the best trade-off of speed and response amplitude. Performance as quantified in these terms did not systematically vary as a function of linker length or composition, as most constructs clustered around similar values for response amplitude and decay (FIG. 4d) with few outliers .

As a final test that the enhanced affinity of the Arr3A mutant for unphosphorylated opsin was a critical consideration in these fusion proteins, we collected a parallel dataset employing fusions of Rod6A with ArrWT (FIG. 8). We found that although these fusions exhibited faster decay than co-expression of Rod6A and ArrWT, they were still slower than Rod6A-Arr3A fusions, such as Rod6A-10 nm-Arr3A. The slower decay demonstrated by ArrWT compared to Arr3A is consistent with our findings for co-transfection of Rod6A with ArrWT and Arr3A shown in FIG. 2, suggesting the increased affinity of Arr3A for R* contributes to the faster T_{off} observed in Rod6A-Arr3A fusions. We also found that Rod6A-ArrWT showed similar reduction in response amplitudes by 1.7-2.4x compared to co-expression of the two proteins.

### Example 5

Rod opsin mutants with faster meta-II decay were tested to reduce light response lifetime. We examined whether increasing the rate of Schiff base hydrolysis could be used to reduce lifetime of rod opsin response and what impact this has on response amplitude. Several rod opsin mutants with faster meta-II decay have been described. E122Q and I189P, were identified by comparing opsins with naturally different meta-II decay rates, such as cone and rod opsins [Kuwayama et al, 2002, Biochemistry 41, 15245-15252]. Y74F and L59Q are structural modifications conserved in opsins of high altitude species, where selection pressure has driven convergent evolution of opsins with decreased thermal stability [Castiglione et al, 2008, Evolution 72, 170-186]. Finally, 3 conserved tyrosines (Y136F, Y223F, Y306F) and an alanine (A132S, A132L) within the retinal binding pocket have been independently shown to stabilise meta-II confirmation [Goncalves et al, 2010, Proc. Natl. Acad. Sci. U.S.A. 107, 19861-19866]. The increased rate of meta-II decay of these mutants was revealed using in vitro spectroscopic assays of purified protein, and in most cases the impact of these mutants on G protein activation has not been described.

Using immunohistochemistry, we confirmed that all rod opsin mutants were correctly localised in the plasma membrane of Hek293T cells (FIG. 9). However, using live cell assays, we found two rod opsin mutants, Y223F and A132L, were non-functional being unable to cause a detectable change in BRET ratio upon light exposure (FIG. 5a). The remaining opsin mutants were functional, with most displaying response amplitudes attenuated to 24-41% of wildtype control (FIG. 5b), such as L59Q (Wilcoxon signed ranks test compared to RodWT, p = 0.002), E122Q (p = 0.001), A132S (p = 0.002), Y136F (p = 0.002) and I189P (p = 0.004). Y74F had comparable response amplitude to RodWT (p = 0.999), while Y306F had a trend towards response amplitudes 1.13x those of RodWT (p = 0.032).

The pattern of response deactivation across rod opsin mutants was more complex (FIG. 5c). Of the 5 mutants with small amplitude responses, E122Q (p = 0.006) and Y136F (p = 0.014) demonstrated 2.03x and 1.72x faster deactivation compared to Rod opsin WT, with Y74F (p = 0.084) showing a weak trend towards shorter response lifetimes. L59Q (p = 0.921) and A132S (p = 0.232) had more variable responses, but seem broadly comparable to Rod opsin WT, while I189P (p = 0.074) had a trend towards response decay 1.5x as slow as wildtype control.

Unexpectedly, the rod opsin mutant with largest response amplitude, Y306F, showed a faster deactivation (p =0.014), with T_{off} 70% of RodWT level. Indeed, across the panel of rod opsin mutants, there was no correlation (Pearson’s R = - 0.182, p = 0.1153) between response amplitude and decay (FIG. 5d), suggesting targeting spontaneous opsin deactivation may represent a viable approach for improving both temporal resolution and response amplitude.

To explore whether combining targeting arrestin binding and meta-II decay can help further enhance the temporal resolution, we attempted to combine them by applying E122Q, Y74F, Y136F or Y306F mutations to the Rod6A-10nm-Arr3A construct (FIG. 10). We found that response parameters A and T_{off} respectively, were similar for Rod6A-10nm-Arr3A with and without meta-II mutants, regardless of which mutation was used.

### Example 6

We have used a live cell BRET assay of G protein activation to model the temporal resolution of an extopic light response produced by a prototypical animal opsin. We find that opsin-driven responses can be rendered more time-delimited with manipulations designed to enhance either arrestin-opsin interactions or the decay rate of the signalling-active meta-II opsin state. Importantly, while there is a general trend for all our manipulations to also reduce peak response amplitude, there was no simple correlation between amplitude and the rate of response decay.

The BRET assay applied here has previously been used to measure signalling from non-light sensitive GPCRs. We have adapted it by modifying ratio of different assay components to improve the dynamic range. Because it reports the first step in signal cascade (dissociation of heterotrimeric G-protein), it provides a closer measure of the current degree of opsin signalling than alternatives which report downstream response components, such as secondary messengers. However, it is still an indirect measure of opsin activity whose time course will provide an indication of opsin activity filtered by delays in the accumulation and decline of free Gby. The changes in T_{off} induced by our manipulations should be viewed in this context. As there need not be a simple linear relationship between T_{off} and the rate of decay of signalling active opsin, quantitative extrapolations from the data collected here to the lifetime of signalling opsin are not possible. Nevertheless, the differences we observe confirm that the assay is able to reveal changes in the lifetime of activated opsin (at least over the range explored here) and to place them in order of magnitude relative to one another and a suitable control.

The optimal opsin for optogenetic purposes is one with very efficient G-protein activation (ensuring high sensitivity and response amplitude) but a short activation lifetime (ensuring high spatiotemporal resolution). A reasonable concern at the outset of these experiments was that these two parameters would be so closely correlated as to render them inseparable. When lifetimes for activated opsin become very short, this parameter may reasonably impact peak amplitude for any response with a reasonable integration time constant. Accordingly, we do find a strong overall tendency for the manipulations employed here to reduce signalling lifetime to also impact peak response amplitude. However, there is substantial variation in amplitude for conditions with similar Toff (and vice versa) indicating that this relationship is not deterministic. An alternative interpretation of the trend towards attenuated T_{off} and A is that any manipulation of a native protein is likely to reduce A and that the association with T_{off} arises because our manipulations were targeted to achieve that effect. In any event, it is certainly the case that, comparing across interventions, it is possible to identify options which more or less approach the optimum of high A and low T_{off}.

The most conceptually straightforward way to limit the signaling lifetime of animal opsins is to enhance interactions with arrestin. Like all GPCRs, opsins are deactivated in a two-step process in which phosphorylation facilitates binding to arrestin, which inhibits signaling. We identified an important challenge in applying this strategy to opsins in heterologous expression in the form of a strong suppressive effect on peak light response amplitude when expressing the kinase required for phosphorylation (GRK1). This is consistent with the known suppressive effects of GRK1 on rod opsin activity, revealed as larger amplitude responses in GRK1-/- mice [Chen et al, 2019, Proc Natl Acad Sci U S A 96, 3718-3722.] and a biphasic response (large initial response, followed by rapid decrease to more sustained lower level after phosphorylation) in wildtype visually intact animals [Imai et al, 2007, J Biol Chem 282, 6677-6684].

Native photoreceptors possess specialised structures, which increase opsin density in order to maximise photon capture. In comparison, opsin photosensitivity is strongly reduced for optogenetic applications, because target cells lack these morphological adaptations, making GRK1-induced reduction in response amplitude likely undesirable. Fortunately, we were able to overcome this problem by applying arrestin mutants with enhanced affinity for unphosphorylated active receptor. These effectively reduced response lifetime in the absence of GRK1, avoiding the suppressive effects of GRK1 expression on response amplitude. Application of the arrestin mutants brings the additional benefit of simplicity. For optogenetic applications, in which packaging size for vectors is often limiting, introducing two proteins (opsin+arrestin) is much more feasible than introducing three (opsin + arrestin + kinase).

We found that tethering Arr3A to the opsin protein allowed faster response decay. This strategy may also mitigate potential problems associated with employing the phosphorylation-independent arrestin mutants. First, tethering to opsin is expected to reduce the potential of these introduced arrestins to interfere with native G-protein-coupled receptors and their separate signalling cascades. Second, overexpression of Arr3A mutant in mouse rods caused photoreceptor degeneration indicating that it may be cytotoxic [Song et al, 2013, Cell Signal, 25]. The deleterious effects of this mutant in photoreceptors is believed to be due to reduced self-association of Arr3A, leading to high concentration of monomer units which interact with signalling pathways that can cause apoptosis [Samaranayake et al, 2018, Front Mol Neurosci, 11]. To what extent the cytotoxicity of these mutants is unique to rod photoreceptors, which have unique morphology and protein expression (possessing 1000-10000 more signalling proteins) [Pugh & Lamb, 2000, Chapter 5 in Handbook of Biological Physics, Molecular Mechanisms in Visual Transduction. North-Holland, pp. 183-255.], is unclear. Tethering the arrestin to rod opsin may limit the off-target effects of arr3A and ameliorate potential cytotoxicity.

Altering the opsin protein to increase the rate of meta-II decay was also an effective strategy for reducing response lifetime. We tried a variety of rod opsin mutants thought to destabilise the Schiff base linking the retinaldehyde chromophore to the opsin protein moiety and increase rate of Schiff base hydrolysis in the G protein signalling meta-II state [Heck et al, 2003, J Biol Chem 278, 3162-3169]. In the absence of signalling termination mechanisms, this spontaneous deactivation is relatively slow, on the order of minutes. We tested a range of opsin mutants that had previously been characterised using spectroscopic methods and were reported to cause a variety of changes to rate of Rod WT meta-II decay, from relatively small 1.2-5 fold increases of Y74F, Y136F and Y306F [Goncalves et al, 2010, Proc. Natl. Acad. Sci. U.S.A. 107, 19861-19866] to more dramatic 4-8 fold change of A132L, E122Q and Y223F [Goncalves et al, 2010, Proc. Natl. Acad. Sci. U.S.A. 107, 19861-19866; Kuwayama et al, 2002, Biochemistry 41, 15245-15252; Imai et al, 2007, J Biol Chem 282, 6677-6684]. We found that two opsin mutants, A132L and Y223F, showed no detectable change in BRET ratio. Since both these mutants are known to have very fast meta-II decay and appear to express normally, we speculate that their deactivation is too fast for efficient G protein activation.

The fastest opsin mutant measured using the BRET assay was E122Q, with ~2-fold faster deactivation than Rod WT. E122Q has previously been shown to restore fast single photon responses when expressed in rod photoreceptors of transgenic mice with genetic ablation of arrestin [Imai et al, 2007, J Biol Chem 282, 6677-6684], suggesting this mutant can be safely expressed in the retina and may be suitable for ectopic expression in other cell types as an improved optogenetic tool. We did not find a straightforward relationship between response amplitude and Toff across these opsin mutants, with some fast meta-II decay mutants such as A132L demonstrating small amplitude relatively slow BRET responses, while mutants with more moderate increase in meta-II decay, such as Y306F, had large amplitude responses. Our findings suggest the relationship between rate of retinal release and G protein activation is complex, highlighting the advantage of using live cell assays as a preliminary screen to identify which opsin mutants might be appropriate for optogenetic control for Gi/o signalling.

Our data suggest a few general rules for manipulating signalling lifetime for animal opsins in live cell environments. Firstly, both arrestin interaction and meta-II decay are suitable targets for manipulation. In our hands, we found that the magnitude of effects on Toff were larger for manipulations targeting arrestin interactions than meta-II decay. Nonetheless, as enhanced meta-II decay could improve the rate of bleach recovery, this may be an important outcome in itself, at least in opsins that lack intrinsic bleach recovery mechanisms. Furthermore, we have not fully explored the potential for additive effects of enhancing both meta-II decay and arrestin interaction. Secondly, phosphorylation independent arrestin variants effectively enhance signal termination while avoiding reductions in amplitude caused by GRK1 expression. They also reduce the number of heterologous proteins to be expressed. Application of the arrestin mutants can be combined with phosphorylation incompetent opsins, which are less likely to interact with native kinases/arrestins, allowing greater control over signalling properties. Thirdly, fusion proteins between opsin and arrestin are a useful strategy to reduce Toff, while reducing potential off-target effects of arrestin overexpression. Finally, there is scope to reduce Toff independent of peak response amplitude, highlighting the importance of careful analysis of potential interventions for optogenetic applications. In these experiments, the optimum trade-off between maintaining peak response and reducing response lifetime was obtained by tethering Arr3A to the Rod6A mutant with a 10 nm linker.

### Example 7- Methods for Examples 1 to 6

Expression Vector Construction: pDNR-DUAL human rhodopsin kinase (NM_002929) was obtained from DNASU plasmid repository, where it was deposited by the Harvard Institute of Proteomics. pENTR223.1 human rod arrestin (NM_000541) was also obtained from DNASU plasmid repository, where it deposited by the ORFeome collaboration. pcDNA3 human rod opsin (NM_000539.3), pcDNA3 Glo22F and pcDNA3 GsO plasmids were as described previously (Bailes & Lucas, 2013; Ballister et al., 2018). Human GalphaOA (AH002708) with pertussis toxin resistant Cys352Ser mutation was purchased from the cDNAResource Center (www.cDNA.org). BRET G protein activation assay constructs - pcDNA3 splitVenus-Gbeta1 (sVβ1), pcDNA3 splitVenus-Ggamma2 (sVy2) and pcDNA3 mGRK3-nLuc - were as described previously [Masuho et al, 2015, Sci Signal 8, ra123] and were generously provided by Prof. Kiril Martemyanov (Scripps Research Institute). Where necessary, ORFs were cloned into pcDNA3 vector using Gibson assembly.

Phosphorylation-independent mouse arrestin1 mutants were adapted from [Vishnivetsky et al., 2013, J. Biol. Chem. 288, 3394-3405] for human arrestin1. Arrestin 3A mutant was created by introducing the following mutations - L377A, V378A and F379A - into pcDNA3 Arrestin using Quikchange Lightning site-directed mutagenesis kit (Agilent) with following primers (Arr1 3A Fwd 5′ - GTTATCAGGATGCAAATgcAgcTgcTGAGGAGTTTGCTCGCC (SEQ ID NO. 7) and Arr1 3A Rev 5′ - GGCGAGCAAACTCCTCAgcAgcTgcATTTGCATCCTGATAAC (SEQ ID NO. 8). The ArrestinKEQ3A mutant was created by introducing additional mutations - K261Q, E350H and Q332K - to pcDNA3 Arrestin 3A using Quikchange Multi Site-directed mutagenesis kit (Agilent) using following primers (K261Q Fwd 5′ -CGAGTGATTATTACGTCcAGCCCGTGGCTATGGAG (SEQ ID NO. 9), K261Q Rev 5′ -CTCCATAGCCACGGGCTgGACGTAATAATCA CTCG (SEQ ID NO. 10), Q332K Fwd 5′ -GAATCCTGGTGTCTTACaAGATCAAGGTGAAGCTCAC (SEQ ID NO. 11), Q332K Rev 5′ GTGAGCTTCACCTTGATCTtGTAAGACACCAGGATTCC (SEQ ID NO. 12), E350H Fwd 5′ -GAGAGCTCACCTCCAGTcAcGTCGCCACTGAGGTCC (SEQ ID NO. 13), E350H Rev 5′ -GGACCTCAGTGGCGACgTgACTGGAGGTGAGCTCTC (SEQ ID NO. 14).

For construction of Rhodopsin6A expression vector: A 246bp DNA fragment was synthesised by Thermo Fisher, which corresponded to the final 196bp of the human Rhodopsin ORF and a 50bp overlap with pcDNA3 backbone from NotI site. This fragment possessed 6 mutations (S333A, T336A, S338A, T340A, T342A, S343A) designed to remove phosphorylation sites from the Rhodopsin C-terminus. This fragment was cloned into pcDNA3 Rhodopsin vector linearized with AfeI and NotI using Gibson Assembly.

Arrestin3A or Arrestin KEQ3A were cloned in-frame after Rhodopsin6A by amplifying Arrestin ORF using following primers (Arr Fwd 5′ -ccaggtggccccggcTaCGCGtGCAGCCAGCGGGAAG ACCAGC (SEQ ID NO. 15) and Arr Rev 5′ -caggaattcgatatcaagcACCGGTTTACTCATCAACGTCATTCTTGTC TCTC (SEQ ID NO. 16). The forward primers introduced a 6 bp MluI restriction site between Rhodopsin6A and Arrestin mutant coding sequences.

To construct bicistronic vectors or fusion constructs between Rhod6A and Arr3A/ArrKEQ3A, DNA sequences corresponding to desired linker or P2A sequence were synthesised by Thermo Fisher and cloned into pcDNA3 Rhod6A-MluI-Arr3A or pcDNA3 Rhod6A-MluI-ArrKEQ3A linearised by MluI digest and treated with recombinant Shrimp Alkaline Phosphatase (NEB) using Gibson assembly.

The following linkers were used in fusion constructs: Flexible linkers composed of (GGGGS)n units where n = 1-3 (Referred to as F1-3, respectively) (SEQ ID NO. 17; SEQ ID NO. 18; SEQ ID NO. 19), Rigid linkers composed of A(EAAAK)nA units where n = 1-3 (Referred to as R1-3, respectively) (SEQ ID NO. 20; SEQ ID NO. 21; SEQ ID NO. 22), Semi-Flexible linkers composed of GGGGSA(EAAAK)nAGGGGS where n = 1-5 (referred to as SF1-5 respectively) (SEQ ID NO. 23; SEQ ID NO. 24; SEQ ID NO. 25; SEQ ID NO. 26; SEQ ID NO. 27). We also tested fusion constructs using the 10 nm and 20 nm E/RK α-helix linkers described previously [Sivaramakrishnan & Spudich, 2011, Proc. Natl. Acad. Sci. U.S.A. 108, 20467-20472]. For the E/RK α-helix linkers, an additional (GSG)4 motif (SEQ ID NO. 28) was included at the 5′ and 3′ end of E/RK linker to ensure rotational freedom.

Presence of mutations and cloned ORFs was confirmed by Sanger sequencing.

### Cell Culture and Transfections

Hek293T cells (ATCC) were incubated at 37 oC (5% CO2) in culture media (Dulbecco’s modified Eagle’s medium with 4500 mg/L glucose, L-glutamine, sodium pyruvate and sodium bicarbonate from Sigma) and 10% fetal bovine serum (FBS).

For transfections, cell were seeded into 12-well plates at a density of 250 000 cells/well in culture medium. After 48 hrs, cells were transiently transfected using Lipofectamine 2000 (Thermo Fisher) according to manufacturer’s instructions. For all transfections, total amount of DNA was normalised between conditions using empty vector.

For BRET G protein activation assays described in FIGS. 1, 2, 6 and 7, each well of 12-well plate was transiently transfected with following:100ng sVβ1, 100 ng sVy2, 100 ng mGRK3-nLuc, 200 ng Gαo, 500 ng opsin and where appropriate 500 ng arrestin (or arrestin mutant) and/or 500 ng rhodopsin kinase. The ratio and amount of BRET assay components was as described in [Masuho et al, Methods Mol Biol 1335, 107-113]

After completing these initial experiments, we optimised the amount and ratio of each of the BRET assay components to improve reliability of assay. We did this by measuring BRET ratio of different levels of mGRK3-nLuc in isolation, then for optimised GRK3-nLuc in combination with different amounts of sVβ1 and sVy2 and finally different ratios of Gαo to optimised amounts of sVβ1, sVy2 and mGRK3-nLuc. For these optimised transfection conditions, used to collect data shown in FIGS. 4, 5, S3 and S5, each well of 12-well plate was transfected with following: 100 ng sVβ1, 100 ng sVy2, 25 ng mGRK3-nLuc, 50 ng Gαo and 500 ng opsin or opsin-arrestin fusion.

For all BRET experiments, once transfected subsequent steps were conducted under dim red light. After addition of transfection reagent and DNA cells were incubated for 4-6 hours at 37 oC, then resuspended in 1 ml of culture media containing 10 µM 9-cis retinal (Sigma-Aldrich). 100 µl of resuspended cells was added to each well of a white-walled clear-bottomed 96-well plate (Greiner Bio-One) and left overnight before performing BRET G protein activation assay.

For Glosensor cAMP assay, each well of 12-well plate was transfected with following:500 ng Opsin (or Opsin-Arrestin fusion), 500 ng Glo-22F, 5 ng GsO and where appropriate 500 ng arrestin (or arrestin mutant) and/or 500 ng rhodopsin kinase. After addition of transfection reagent and DNA cells were incubated for 4-6 hours at 37 oC, then resuspended in 1 ml of culture media containing 10 µM 9-cis retinal and 125 ng/ml pertussis toxin. 100 µl of resuspended cells was added to each well of a white-walled white-bottomed 96-well plate (Greiner Bio-One) and left overnight before performing cAMP assay.

### Example 8

BRET G protein activation assay: Approximately 1-2 hours before beginning BRET G protein activation assay, culture media was removed from cells and replaced with 50 µl imaging media (L-15 media without Phenol Red containing L-glutamine (Gibco), 1% FBS, penicillin (100 U/ml) and streptomycin (100 µg/ml) with 10 µM 9-cis retinal. Cells were then left to incubate at room temperature in dark for at least 1 hour.

Under dim red light, NanoGlo Live Cell substrate (Furimazine derivative, Promega) was diluted 1:40 in PBS. Then 12.5 ul of dilute NanoGlo substrate solution was added to each well of 96-well plate (to provide final dilution of 1:200 of NanoGlo substrate), for up 6 wells at a time, and incubated for 5 mins before commencing assay to allow luminescence to peak. We found recordings conducted more than 20 mins after cells were initially loaded with substrate tended to be noisier as overall BRET signal decreased.

BRET measurements were conducted using a FluoStar Optima microplate reader (BMG Labtech). As this plate reader has a single photomultiplier tube, light emitted by fluorescent Venus and bioluminescent Nanoluc were measured sequentially using 535 nm (30 nm FWHM with gain set to 4095) and 470 nm (30 nm FWHM with gain set to 3600) emission filters. A 0.68 s recording interval was used for each filter, with a total cycle time of 2 s.

To avoid delays associated removing plate from reader for light exposure, we adapted the plate reader bottom optic to allow us to deliver light to individual wells inside the plate reader. A custom 3D-printed coupler was used to connect the bottom optic with the liquid light guide of a Lumencor SpectraX light engine. Combined with clear-bottomed 96-well plates, this allowed us to provide a light stimulus below cells. To avoid bleaching the PMT during light stimulus, a motorized shutter was built to protect the PMT by blocking top optic light path while light stimulus was on. The activity of this shutter was synced to the light source using an Arduino microcontroller. To avoid neighbouring wells being exposed to light, each recorded well was surrounded by empty wells and the order of wells measured was counterbalanced.

During BRET plate reader recordings, using Optima script mode, a 14.5 second pause followed by a 1 s 470 nm light pulse (14.1 log photons) was first triggered by an executable file. A well-mode protocol for the individual well to be recorded was then immediately started. This protocol consisted of 2 kinetic windows; the first consisted of 5 cycles of baseline measurement (total duration 10 s) and the second protocol began after a short 3-4 s delay (during which cells were exposed to the delayed light pulse), before continuing for up to 45 cycles (total duration 90 s). The pause between the two kinetic windows was varied according to the well position being recorded in order to account for different delays in time taken to travel from plate reader “home position” and ensure recording was resumed immediately after light flash. This process was then repeated for until all wells loaded with substrate had been measured.

In each recording session, between 3-4 repeats were conducted for all conditions. At least 3 recording sessions (each a separate transfections) were performed for each experiment.

Glosensor Gso cAMP assay: Glosensor Gso assay was performed as described previously (Ballister et al, 2018). Briefly, 1-2 hours before beginning assay - cells were incubated at room temperature in 75 ul imaging media with 2 mM beetle luciferin potassium salt (Promega) reconstituted in 10 mM HEPES pH 6.9. Using the FluoStar Optima microplate reader, raw luminescence was recorded using 3 mm lens (Gain set to 3600) for 1 s, every 60 s. Baseline luminescence was recorded for 5 cycles, then recording was paused and plate ejected. Each well was then stimulated with 470 nm light flash using a custom-built LED array. Each well was exposed to one of eight different intensities over a 5-log range (from 4x1012 to 1016 photons). One well from each condition was left unexposed as a dark control.

For BRET G protein activation assay - BRET signal was determined by calculating ratio of light emitted by Venus-Gβ1γ2 at 535 nm with light emitted by mGRK3-nLuc at 470 nm. The BRET signal was then normalised to baseline by dividing each time point by the last baseline value before stimulus to give ΔBRET ratio. The kinetics of ΔBRET ratio time course post-stimulus were then fit to the following 3-parameter model using non-linear regression:

y =e(-Toff / x) ▪ (1- e(-Ton / x)) ▪ A

where Toff = rate of decay of exponential decay curve, Ton = rate of increase of exponential association curve and A = scaling factor of two exponential curves, x = time (seconds) and y = baseline normalised BRET signal. Model was fit to data using non-linear regression. The following constraints were used: A > 0, Ton > 0.1, Toff > 5, R2 > 0.2. Curve fits that were ambiguous or did not converge were excluded from analysis and are not included in time courses displayed in figures (except in FIG. S5 where all data is displayed).

Scaling factor (A) was used as a measure of response amplitude, while Toff (s) was used as a measure of response decay. We found systematic variation in overall ΔBRET ratio of different recordings. To account for this, we looked at fold change in A and Toff of each condition relative to RodWT positive control conducted as part of same repeat. Response amplitude and decay were then analysed using Wilcoxon Signed Ranks Test, comparing each group to theoretical median = 1.

Immunocytochemistry: Hek293 cells were seeded into 12-well plates at a density of 250 000 cells/well in culture medium. After 48 hrs, cells were transiently transfected using Lipofectamine 2000 (Thermo Fisher) according to manufacturer’s instructions with 500 ng opsin or opsin-arrestin fusion. Cells were incubated at 37 oC for 4-6 hours and then, under dim red light, resuspended in 2 ml of culture media containing 10 µM 9-cis retinal (Sigma-Aldrich). The entire volume of resuspended cells was then added to a well of a 6-well plate containing 3 x poly-D-lysine coated glass coverslips. Cells were incubated for a further 24 hours then washed once with PBS, before being fixed using 4% paraformaldehyde in PBS. Cells were then washed three times in PBS and stored in PBS at 4oC until being stained.

For staining, one coverslip per condition was removed and placed in each well of 12-well plate. Cells were permeabilised in 0.2% Triton-X in PBS for 5 mins, then blocked in PBS + 0.05% Tween-20 with 5% serum for 20-30 mins. Cells were incubated in primary antibodies diluted in PBS + 0.05% Tween-20 + 1% serum for 1 hour at room temperature, then washed three times in PBS. Cells were then incubated in secondary antibody diluted in PBS + 0.05% Tween-20 + 1% serum for 30 mins at room temperature in dark. Cells were washed in PBS 3 more times, then each coverslip was mounted onto slides using Prolong Gold anti-fade media with DAPI and allowed to dry at room temperature for at least 24 hours.

The following primary and secondary antibodies were used: Mouse monoclonal anti-4D2 N-terminal rod opsin antibody 4D2 (1:500, Abcam, Ab98887) with Donkey anti-mouse far red 594 secondary (1:500, Molecular Probes) with donkey serum; Mouse monoclonal anti-1D4 C-terminal rod opsin antibody (1:500, Abcam, Ab5417) with Goat anti-mouse red 555 secondary (1:500, Molecular probes) with Goat Serum.

Images were acquired using an Axio Imager.D2 Upright microscope (Zeiss) using a 40X plan neofluar air objective, using excitation at 350 nm, 470 nm and 580 nm, and emission at 460 nm, 515 and 650 nm for DAPI, green and red fluorescence, respectively. Images were collected using Micromanager v1.4.23, with a Coolsnap HQ2 camera (Photometrics) and CoolLED pE-300 White light source. Images were analysed using FIJI ImageJ (Schneider et al., 2012)]. Global adjustments to brightness and/or contrast were applied equally to all images.

### Example 8

We have used adeno-associated virus containing one of the following optogenetic transgenes (FIG. 11):
1. Rod6A-10nm-Arrestin3A - This is phosphonull human rod opsin mutant fused to a phosphorylation-independent visual arrestin mutant by a semi-flexible linker.
2. Rod opsin E122Q - This is human rod opsin with a non-synonymous point mutation that causes faster decay of meta-II signalling state. The smaller size of this transgene allows us to co-express with a fluorescent mCherry reporter.
3. Rod opsin WT - This is wildtype human rod opsin, used as a positive control for MEA experiments. This transgene was co-expressed with an mCherry fluorescent reporter.

The virus is floxed, meaning the optogenetic transgene is only expressed in expressing Cre-recombinase. We gave Grm6Cre rd1 mice bilateral intravitreal injections of one of the viruses. These mice are retinally degenerate and express Cre-recombinase under control of the Grm6 promoter exclusively in the rod ON bipolar cells.

We performed multi-electrode array recordings on retinal explants from injected animals in response to 1 s of white light across a range of intensities (12.5 - 15.5log rod opsin effective photons/cm²/s). Retinas were excised and placed ganglion cell side down onto a multielectrode array to record spiking activity from the ganglion cell population.

We found retinally degenerate retinas transduced with Rod6A-10 nm-Arr3A had transient excitatory responses to light between 15.5 down to 13.5log photons/cm²/s (FIG. 12).

We saw similar transient excitatory responses in retinas transduced with Rod opsin E122Q (FIG. 13). The fluorescent reporter mCherry was widely expressed throughout the retina, suggesting viral transduction was successful (FIG. 14).

There appeared to be a qualitative difference in the nature of the light evoked activity achieved with the rod opsin variants designed to improve temporal resolution. Importantly, in each case, responses to a light flash were less sustained (more closely linked to the time of light appearance) in retinas expressing either Rod opsin E122Q or Rod6A-10 nm-Arr3A, compared to unmodified Rod opsin (FIG. 15).

Both rod opsin E122Q and Rod6a-10 nm-Arr3A supported light-evoked activity when expressed in ON bipolar cells of mouse retina. Both appeared to have improved temporal response characteristics compared to native rod opsin.

### Methods for Example 8

### Animals

All experiments and care was conducted in accordance with the UK Animals (Scientific Procedures) Act (1986). Grm6^{+/Cre} (Morgan, C.W. et al, (2009). Proc Natl Acad Sci USA, 106 (45), 19174-8, doi: 10.1073/pnas.0908711106) rd1 mice on a mixed C3H x C57Bl/6 background were used. These mice have Cre recombinase expressed under control of the Grm6 (mGlur6) promoter, resulting in restricted expression of Cre in rod bipolar cells. They also possess the Pde6b^{rd1} mutation Chang, et al (2002) Vision Research, 42(4), 517-525. https://doi.org/10.1016/S0042-6989(01)00146-8 and Pittler, S. J., & Baehr, W. (1991) Proceedings of the National Academy of Sciences of the United States of America, 88(19), 8322-8326. https://doi.org/10.1073/pnas.88.19.8322), which causes progressive retinal degeneration with vision loss complete once animals are over 80 days old. Mice were genotyped to confirm they do not possess the GRP179 point mutation (Peachey et al. (2012) American Journal of Human Genetics, 90(2), 331-339. https://doi.org/10.1016/j.ajhg.2011.12.006) that affects bipolar cell function. Mice were kept under a 12:12 light dark cycle with food and water provided ad libitum. Multi-electrode array recordings were conducted between 13 to 15 weeks after bilateral intraocular injection of adeno-associated virus at 9-10 weeks old.

### Intravitreal Injections

Grm6^{Cre/+} rd1 mice received bilateral intravitreal injections of virus (AAV2 4YF- ITR - DIO-CMV-Rod opsin WT-T2A-mCherry - WPRE- SV40 late polyA - ITR, or AAV2 4YF- ITR - DIO-CMV-Rod opsin E122Q-T2A-mCherry - WPRE- SV40 late polyA - ITR, or AAV2 4YF- ITR - DIO-CMV-Rod6A-10nm-Arr3A- WPRE- SV40 late polyA - ITR). The virus was packaged in an AAV2/2 capsid with four tyrosine to phenylalanine mutations ( Petrs-Silva et al (2011) Molecular Therapy: The Journal of the American Society of Gene Therapy, 19(2), 293-301. https://doi.org/10.1038/mt.2010.234) to achieve efficient viral transduction of retinal cells, in particular bipolar cells. The Rod Opsin WT and Rod Opsin E122Q transgenes were linked to a mCherry fluorescent reporter using a T2A sequence to ensure 1:1 co-expression of the two proteins. The open reading frame of the inverted optogenetic transgene (and fluorescent reporter, where applicable) was flanked by two pairs of Lox sites (LoxP and Lox2272), so that in the presence of Cre recombinase, the transgene is inverted into the sense orientation and expression is driven by the constitutive CMV (cytomegalovirus) promotor. A woodchuck hepatitis virus post-transcriptional regulatory element (WPRE) and SV40 late polyA sequence were also included between ITRs to improve transgene expression. Virus was obtained from VectorBuilder.

Mice were anaesthetised by intraperitoneal injection ketamine (75 mg/kg body weight) and medetomidine (1 mg/kg body weight). Once anaesthetised, mice were positioned on a heat mat to prevent cooling. Pupils were dilated with 1% tropicamide eye drops (Bausch & Lomb) and a 13 mm coverslip was positioned on gel lubricant (Lubrithal) applied to the cornea. Between 2.2-2.5 ul of virus (1.12 × 10¹³ genomic counts per ml) was injected into the vitreous of each eye using a Nanofil 10µl syringe (World Precision Instruments) using 35-gauge bevelled needle using a surgical microscope (M620 F20, Leica). All mice received bilateral injections. Anaesthesia was reversed by intraperitoneal injection of atipamezole (3 mg/kg body weight). During recovery, 0.5% bupivacaine hydrochloride and 0.5% chloramphenicol was applied topically to the injected eyes. Mice also received 0.25 ml of warm saline given by subcutaneous injection to aid recovery.

### Multi-Electrode Array Recordings

Mice were dark adapted overnight. All following steps were performed under diffuse dim red light. Dark adapted mice were culled by cervical dislocation (approved Schedule 1 method). Enucleated eyes were placed in petri dish filled with carboxygenated (95% O₂/ 5% CO₂) Ames’ media (supplemented with 1.9 g/L sodium bicarbonate, pH 7.4, Sigma Aldrich) and retinas dissected, with care taken to remove vitreous from inner retinal surface. Retinal wholemounts were then placed on glass coated metal harps (ALA Scientific Instruments), and positioned ganglion-cell side down on coated 256-channel multi-electrode arrays (MEA, Multi Channel Systems). Multi-electrode arrays were first incubated in fetal bovine serum overnight at 4° C., then coated with 0.1% polyethyleneimine (PEI) in borate buffer (pH8.4) for 1 hr at room temperature. PEI coating was then removed, and MEA washed 4-6 times with ddH₂0. PEI-coated MEAs were then air-dried and coated with 20 µg/ml laminin in fresh Ames’ medium for 30-45 mins at room temperature ( Egert, U., & Meyer, T. (2005), In S. Dhein, F. W. Mohr, & M. Delmar (Eds.), Practical Methods in Cardiovascular Research (pp. 432-453). https://doi.org/10.1007/3-540-26574-0_22, 2005; Lelong, et al (1992). Journal of Neuroscience Research, 32(4), 562-568. https://doi.org/10.1002/jnr.490320411). Laminin solution was removed before retina was positioned on the MEA. Once in place on the MEA, the retina was continuously perfused with carboxygenated Ames’ media with 10 µM 9-cis retinal at 2-3 ml/min using a peristaltic pump (PPS2, Multi Channel Systems) and maintained at 34° C. using a water bath heater (36° C.), inline perfusion heater (35° C.) and base plate heater (34° C.). Once positioned, retinas were perfused in dark for at least 45mins before first light stimuli were applied. Data were sampled at 25 kHz using MC Rack software (Multi Channel Systems). A Butterworth 200 Hz high pass filter was applied to raw electrode data to remove low frequency noise. Amplitude threshold for spike detection was 4-4.5 standard deviations from baseline. Light stimuli were presented using a customised light engine (Thorlab LEDs). An Arduino Due microcontroller controlled by programmes written in LabVIEW (National Instruments) to control stimulus duration and intensity by altering LED output.

### Fluorescence Microscopy

For images of retinal flatmounts on multi-electrode array, once recording was complete, media was drained from MEA chamber and metal harp removed. The entire MEA chamber was then placed on microscope stage and images of mCherry fluorescence from transduced cells in retinal wholemount were acquired.Images of mCherry immunostaining and fluorescence were acquired using a Leica DM2500 microscope with DFC365 FX camera (Leica) and a CoolLED-pE300-white light source. Imaging software was Leica Application Suite Advanced Fluorescence6000. Images were acquired using Chroma ET Y3 filter set (excitation = 545 nm, emission = 610 nm). Global enhancements to image brightness and contrast were made using ImageJ software.

Table 1. Sequence Listing - open reading frames of Arrestin 3A, Arrestin KEQ3A, GRK1, Rod6A, Human wild type opsin and wild type arrestin. The first nucleotide is residue 1 of the ORF. Herein, point mutations are numbered with reference to nucleotide 1 of the ORF.

| | |
|---|---|
| SEQ. ID. No. | Sequence |
| 1 (Arrestin 3A) | ATGGCAGCCAGCGGGAAGACCAGCAAGTCCGAACCGAACCATGTTATCTTCAAGAAGATCTCCCGGGACAAATCGGTGACCATCTACCTGGGGAACAGAGACTACATAGACCATGTCAGCCAAGTCCAGCCTGTGGATGGTGTCGTGTTGGTTGATCCTGATCTTGTGAAGGGAAAGAAAGTGTATGTCACTCTGACCTGCGCCTTCCGCTATGGCCAAGAGGACATTGACGTGATCGGCTTGACCTTCCGCAGGGACCTGTACTTCTCCCGGGTCCAGGTGTATCCTCCTGTGGGGGCCGCGAGCACCCCCACAAAACTGCAAGAGAGCCTGCTTAAAAAGCTGGGGAGCAACACGTACCCCTTTCTCCTGACGTTTCCTGACTACTTGCCCTGTTCAGTGATGTTGCAGCCAGCTCCACAAGATTCAGGGAAGTCCTGTGGGGTTGACTTTGAGGTCAAAGCATTCGCCACAGACAGCACCGATGCCGAAGAGGACAAAATCCCCAAGAAGAGCTCCGTGCGATTACTGATCCGCAAAGTACAGCATGCCCCACTTGAGATGGGTCCCCAGCCCCGAGCTGAGGCGGCCTGGCAGTTCTTCATGTCTGACAAGCCCCTGCACCTTGCGGTCTCTCTCAACAAAGAGATCTATTTCCATGGGGAGCCCATCCCTGTGACCGTGACTGTCACCAATAACACAGAGAAGACCGTGAAGAAGATTAAAGCATTCGTGGAACAGGTGGCCAATGTGGTTCTCTACTCGAGTGATTATTACGTCAAGCCCGTGGCTATGGAGGAAGCGCAAGAAAAAGTGCCACCAAACAGCACTTTGACCAAGACGCTGACGCTGCTGCCCTTGCTGGCTAACAATCGAGAAAGAGAGGCATTGCCCTGGATGGGAAAATCAAGCACGAGGACACAAACCTTGCCTCCAGCACCATCATTAAGGAGGGCATAGACCGGACCGTCCTGGGAATCCTGGTGTCTTACCAGATCAAGGTGAAGCTCACAGTGTCAGGCTTTCTGGGAGAGCTCACCTCCAGTGAAGTCGCCACTGAGGTCCCATTCCGCCTCATGCACCCTCAGCCTGAGGACCCAGCTAAGGAAAGTTATCAGGATGCAAATgcAgcTgcTGAGGAGTTTGCTCGCCATAATCTGAAAGATGCAGGAGAAGCTGAGGAGGGGAAGAGAGACAAGAATGACGTTGATGAGTAA |
| 2 (Arrestin KEQ3A) | ATGGCAGCCAGCGGGAAGACCAGCAAGTCCGAACCGAACCATGTTATCTTCAAGAAGATCTCCCGGGACAAATCGGTGACCATCTACCTGGGGAACAGAGACTACATAGACCATGTCAGCCAAGTCCAGCCTGTGGATGGTGTCGTGTTGGTTGATCCTGATCTTGTGAAGGGAAAGAAAGTGTATGTCACTCTGACCTGCGCCTTCCGCTATGGCCAAGAGGACATTGACGTGATCGGCTTGACCTTCCGCAGGGACCTGTACTTCTCCCGGGTCCAGGTGTATCCTCCTGTGGGGGCCGCGAGCACCCCCACAAAACTGCAAGAGAGCCTGCTTAAAAAGCTGGGGAGCAACACGTACCCCTTTCTCCTGACGTTTCCTGACTACTTGCCCTGTTCAGTGATGTTGCAGCCAGCTCCACAAGATTCAGGGAAGTCCTGTGGGGTTGACTTTGAGGTCAAAGCATTCGCCACAGACAGCACCGATGCCGAAGAGGACAAAATCCCCAAGAAGAGCTCCGTGCGATTACTGATCCGCAAAGTACAGCATGCCCCACTTGAGATGGGTCCCCAGCCCCGAGCTGAGGCGGCCTGGCAGTTCTTCATGTCTGACAAGCCCCTGCACCTTGCGGTCTCTCTCAACAAAGAGATCTATTTCCATGGGGAGCCCATCCCTGTGACCGTGACTGTCACCAATAACACAGAGAAGACCGTGAAGAAGATTAAAGCATTCGTGGAACAGGTGGCCAATGTGGTTCTCTACTCGAGTGATTATTACGTCcAGCCCGTGGCTATGGAGGAAGCGCAAGAAAAAGTGCCACCAAACAGCACTTTGACCAAGACGCTGACGCTGCTGCCCTTGCTGGCTAACAATCGAGAAAGGAGAGGCATTGCCCTGGATGGGAAAATCAAGCACGAGGACACAAACCTTGCCTCCAGCACCATCATTAAGGAGGGCATAGACCGGACCGTCCTGGGAATCCTGGTGTCTTACaAGATCAAGGTGAAGCTCACAGTGTCAGGCTTTCTGGGAGAGCTCACCTCCAGTcAcGTCGCCACTGAGGTCCCATTCCGCCTCATGCACCCTCAGCCTGAGGACCCAGCTAAGGAAAGTTATCAGGATGCA AATgcAgcTgcTGAGGAGTTTGCTCGCCATAATCTGAAAGATGCAGGAGAAGCTGAGGAGGGGAAGAGAGACAAGAATGACGTTGATGAGTAA |
| 3 (GRK1) | ATGGATTTCGGGTCTTTGGAGACCGTGGTGGCCAACTCTGCCTTCATCGCCGCCCGAGGCAGCTTTGACGGCAGCAGCTCCCAACCCTCCCGGGACAAGAAGTACCTGGCCAAGCTCAAGCTGCCCCCGCTGTCCAAGTGTGAGTCCCTCCGCGACAGCCTCAGCCTGGAGTTTGAGAGTGTGTGCTTGGAGCAGCCCATCGGCAAGAAGCTCTTTCAGCAGTTCCTACAATCGGCAGAGAAGCACCTGCCGGCCCTGGAGCTCTGGAAAGACATCGAGGACTATGACACGGCAGACAATGACCTCCAGCCACAGAAGGCCCAGACCATCCTGGCCCAGTACCTGGACCCCCAGGCCAAACTCTTCTGCAGCTTCCTGGATGAGGGGATAGTGGCGAAGTTTAAGGAGGGGCCTGTGGAGATCCAGGACGGGCTCTTCCAGCCCCTGCTGCAGGCCACCCTGGCACACCTGGGCCAAGCCCCCTTCCAGGAGTACCTGGGCAGCCTGTACTTCCTGAGGTTCCTGCAGTGGAAGTGGCTGGAAGCCCAGCCCATGGGGGAGGACTGGTTCCTGGACTTCAGGGTCCTGGGGAAAGGGGGCTTCGGGGAGGTGTCGGCCTGCCAGATGAAGGCGACCGGCAAGCTGTATGCCTGCAAGAAGCTGAACAAGAAGCGGCTGAAGAAGAGGAAGGGCTACCAGGGTGCTATGGTGGAGAAGAAGATTCTGATGAAAGTACACAGCAGGTTCATCGTGTCTCTGGCCTATGCGTTTGAAACCAAAGCCGACCTCTGTCTGGTGATGACCATCATGAACGGAGGTGACATCAGGTACCACATCTACAACGTGAATGAGGAGAACCCTGGCTTCCCGGAGCCGCGCGCCCTCTTCTACACGGCGCAGATCATCTGCGGCCTGGAGCACCTGCACCAGAGGCGGATCGTCTACCGCGACCTCAAGCCCGAGAACGTGCTGCTGGACAATGACGGCAATGTCCGGATCTCTGACCTTGGGCTGGCCGTGGAGCTGCTGGACGGACAGAGCAAGACCAAGGGCTACGCAGGGACCCCAGGTTTCATGGCCCCCGAGCTCCTGCAGGGCGAGGAGTACGACTTCTCCGTGGACTACTTTGCCCTGGGGGTCACCCTGTATGAGATGATTGCGGCCAGAGGACCCTTCCGAGCCCGTGGAGAGAAGGTGGAGAACAAGGAGCTGAAGCACCGGATCATCTCAGAGCCCGTGAAGTACCCTGATAAGTTCAGCCAGGCCAGCAAGGACTTCTGCGAGGCGCTGCTGGAGAAGGACCCGGAGAAGCGCCTGGGGTTCAGAGATGAGACCTGCGACAAGCTCCGTGCCCACCCCCTCTTCAAGGACCTTAACTGGAGGCAGCTGGAGGCTGGGATGCTGATGCCCCC TTTCATCCCAGACTCCAAAACTGTCTACGCAAAGGATATTCAGGACGTGGGTGCCTTTTCCACCGTCAAAGGTGTGGCCTTTGACAAAACAGACACAGAATTCTTTCAGGAATTTGCCACTGGCAACTGCCCCATCCCCTGGCAGGAGGAGATGATCGAGACGGGCATCTTTGGCGAGCTGAACGTGTGGCGCTCGGACGGTCAGATGCCGGACGACATGAAGGGCATCTCCGGGGGCTCCAGCTCCTCGTCCAAGTCAGGGATGTGTCTGGTTTCCTAA |
| 4 (Rod6A) | ATGAATGGCACAGAAGGCcctaacttctacgtgcccttctccaatgcgacgggtgtggtacgcagccccttcgagtacccacagtactacctggctgagccatggcagttctccatgctggccgcctacatgtttctgctgatcgtcctaggcttccccataaatttcctcacgctctacgtcaccgtccagcacaagaagctgcgcacgcctctcaactacatcctgctcaacctagccgtggcagatctcttcatggttctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacgtgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctcatatgctgggtgccctacgccagcgtggcattctacatcttcacccaccagggctccaacttcggTCCCATCTTCATGACCATCCCAGCGTTCTTTGCCAAGAGCGCTGCCATCTACAACCCTGTCATCTATATCATGATGAACAAGCAGTTCCGGAACTGTATGCTGACCACCATCTGCTGCGGCAAGAACCCTCTGGGAGATGATGAAGCCGCTGCCGCCGTGGCTAAAGCTGAAGCTGCTCAAGTGGCCCCTGCCTAA |
| 5 Human wild type opsin | atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtcctaggcttccccataaatttcctcacgctetacgtcaccgtccagcacaagaagctgcgcacgcctctcaactacatcctgctcaacctagccgtggcagatctcttcatggttctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacgtgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctcatatgctgggtgccctacgccagcgtggcattctacatcttcacccaccagggctccaacttcggtc ccatcttcatgaccatcccagcgttctttgccaagagcgctgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa |
| 6 Arrestin wild type sequence | ATGGCAGCCAGCGGGAAGACCAGCAAGTCCGAACCGAACCATGTTATCTTCAAGAAGATCTCCCGGGACAAATCGGTGACCATCTACCTGGGGAACAGAGACTACATAGACCATGTCAGCCAAGTCCAGCCTGTGGATGGTGTCGTGTTGGTTGATCCTGATCTTGTGAAGGGAAAGAAAGTGTATGTCACTCTGACCTGCGCCTTCCGCTATGGCCAAGAGGACATTGACGTGATCGGCTTGACCTTCCGCAGGGACCTGTACTTCTCCCGGGTCCAGGTGTATCCTCCTGTGGGGGCCGCGAGCACCCCCACAAAACTGCAAGAGAGCCTGCTTAAAAAGCTGGGGAGCAACACGTACCCCTTTCTCCTGACGTTTCCTGACTACTTGCCCTGTTCAGTGATGTTGCAGCCAGCTCCACAAGATTCAGGGAAGTCCTGTGGGGTTGACTTTGAGGTCAAAGCATTCGCCACAGACAGCACCGATGCCGAAGAGGACAAAATCCCCAAGAAGAGCTCCGTGCGATTACTGATCCGCAAAGTACAGCATGCCCCACTTGAGATGGGTCCCCAGCCCCGAGCTGAGGCGGCCTGGCAGTTCTTCATGTCTGACAAGCCCCTGCACCTTGCGGTCTCTCTCAACAAAGAGATCTATTTCCATGGGGAGCCCATCCCTGTGACCGTGACTGTCACCAATAACACAGAGAAGACCGTGAAGAAGATTAAAGCATTCGTGGAACAGGTGGCCAATGTGGTTCTCTACTCGAGTGATTATTACGTCAAGCCCGTGGCTATGGAGGAAGCGCAAGAAAAAGTGCCACCAAACAGCACTTTGACCAAGACGCTGACGCTGCTGCCCTTGCTGGCTAACAATCGAGAAAGGAGAGGCATTGCCCTGGATGGGAAAATCAAGCACGAGGACACAAACCTTGCCTCCAGCACCATCATTAAGGAGGGCATAGACCGGACCGTCCTGGGAATCCTGGTGTCTTACCAGATCAAGGTGAAGCTCACAGTGTCAGGCTTTCTGGGAGAGCTCACCTCCAGTGAAGTCGCCACTGAGGTCCCATTCCGCCTCATGCACCCTCAGCCTGAGGACCCAGCTAAGGAAAGTTATCAGGATGCAAATTTAGTTTTTGAGGAGTTTGCTCGCCATAATCTGAAAGATGCAGGAGAAGCTGAGGAGGGGAAGAGAGACAAGAATGACGTTGATGAGTAA |

### What Is Paragraphed Is

1. A composition comprising an opsin polypeptide and an arrestin polypeptide, wherein at least one of the opsin polypeptide and the arrestin polypeptide comprises a mutation that increases a temporal resolution of the opsin polypeptide’s response to light.

2. The composition of paragraph 1, wherein the arrestin is a mutant form.

3. The composition of paragraph 1, wherein the arrestin is a wild type.

4. The composition of paragraph 1-3, wherein the opsin is a wild type.

5. The composition of paragraph 1-3, wherein the opsin is a mutant form.

6. The composition of paragraphs 1 or 2, or 4 or 5 when dependent upon paragraphs 1 or 2, wherein the arrestin polypeptide comprises a mutation that allows for phosphorylation independent binding between the opsin polypeptide and arrestin polypeptide.

7. The composition of any one of paragraphs 1 to 6, wherein the opsin polypeptide is operably linked to the arrestin polypeptide through a linker.

8. The composition of paragraph 7, wherein the linker is a flexible linker, a rigid linker, a semi-flexible linker ER/K linker, or a combination thereof.

9. The composition of paragraph 7, wherein the linker is a flexible glycine-serine linker, a rigid alpha-helix forming linkers, a semi-flexible linker having a rigid linker with flexible ends, and a ER/K linker, or a combination thereof.

10. The composition of any one of paragraphs 1 to 6, wherein the opsin polypeptide and the arrestin polypeptide are separate.

11. The composition of any one of paragraphs 1 to 10, wherein the opsin is substantially not phosphorylated by a G-protein coupled receptor kinase.

12. The composition of any one of paragraphs 1 or 2, or 4 to 11 when dependent upon paragraphs 1 or 2, wherein the opsin polypeptide comprises at least one mutation increasing a rate of Schiff base hydrolysis or a rate of meta-II decay.

13. The composition of paragraph 2, wherein the opsin polypeptide comprises at least a mutation selected from the group consisting of L59Q, Y74F, E122Q, A132L, A132S, Y136F, I189P, Y227F, Y306F, and a combination thereof.

14. The composition of any one of paragraphs 1 to 3 or 5, or paragraphs 6 to 13 when dependent upon paragraphs 1 to 3 or 5, wherein the opsin polypeptide comprises a mutation associated with a C-terminal phosphorylation site.

15. The composition of any one of paragraphs 1 to 3 or 5, or paragraphs 6 to 14 when dependent upon paragraphs 1 to 3 or 5, wherein the opsin polypeptide comprises a sequence having at least 70%, 80%, 90%, 95%, or 100% sequence identity to SEQ ID NO. 4.

16. The composition of any one of paragraphs 1, 2 or 4 to 15 when dependent upon paragraphs 1 or 2 wherein the arrestin polypeptide is an Arr3A or ArrKEQ3A mutant.

17. The composition of any one of paragraphs 1, 2 or 4 to 16 when dependent upon paragraphs 1 or 2, wherein the arrestin polypeptide comprises a sequence having at least 70%, 80%, 90%, 95%, or 100% sequence identity to SEQ ID NO. 1.

18. The composition of any one of paragraphs 1, 2 or 4 to 16 when dependent upon paragraphs 1 or 2, wherein the arrestin polypeptide comprises a sequence having at least 70%, 80%, 90%, 95%, or 100% sequence identity to SEQ ID NO. 2.

19. The composition of any one of paragraphs 1, 2 or 4 to 18 when dependent upon paragraphs 1 or 2, wherein the arrestin polypeptide comprises a mutation associated with increased affinity for unphosphorylated opsin.

20. The composition of any one of paragraphs 1, 2 or 4 to 19 when dependent upon paragraphs 1 or 2, wherein the arrestin polypeptide comprises at least a mutation selected from the group consisting of L377A, V378A, F379A, and a combination thereof.

21. The composition of any one of paragraphs 1, 2 or 4 to 20 when dependent upon paragraphs 1 or 2 wherein the arrestin polypeptide comprises at least a mutation selected from the group consisting of L377A, V378A, F379A, K261Q, E350H, Q332K, or a combination thereof.

22. A composition comprising an opsin polypeptide operably linked to an arrestin polypeptide.

23. The composition of paragraph 22, wherein the opsin polypeptide is a wild type or a mutant.

24. The composition of paragraph 22 or paragraph 23, wherein the arrestin polypeptide is a wild type or a mutant.

25. The composition of any one of paragraphs 22 to 24, wherein the linker is a flexible linker, a rigid linker, a semi-flexible linkerER/K linker, or a combination thereof.

26. The composition of any one of paragraphs 22 to 25, wherein the linker is a flexible glycine-serine linker, a rigid alpha-helix forming linkers, a semi-flexible linker having a rigid linker with flexible ends, and a ER/K linker, or a combination thereof.

27. The composition of paragraph any one of paragraphs 22 to 26, wherein opsin polypeptide comprises at least one mutation increasing a rate of Schiff base hydrolysis or a rate of meta-II decay.

28. The composition of paragraph 27, wherein the opsin polypeptide comprises at least a mutation selected from the group consisting of L59Q, Y74F, E122Q, A132L, A132S, Y136F, I189P, Y227F, Y306F, and a combination thereof.

29. The composition of any one of paragraphs 22 to 28, wherein the opsin polypeptide comprises at least one mutation associated with one or more phosphorylation sites.

30. The composition paragraph 29, wherein the mutation is associated with a C-terminal phosphorylation site.

31. The composition of paragraph 29 or 30, wherein the mutation results in amino acid substitution, deletion, or addition at the one or more phosphorylation sites of the opsin polypeptide.

32. The composition of any one of paragraphs 29 to 31, wherein the opsin polypeptide comprises a sequence having at least 70%, 80%, 90%, 95%, or 100% sequence identity to SEQ ID NO. 4.

33. The composition of any one of paragraphs 22 to 28, wherein the arrestin polypeptide having a mutation that increases binding between the arrestin polypeptide and unphosphorylated opsin.

34. The composition of paragraph 33, wherein the arrestin polypeptide comprises a sequence having at least 70%, 80%, 90%, 95%, or 100% sequence identity to SEQ ID NO. 1.

35. The composition of paragraph 33, wherein the arrestin polypeptide comprises a sequence having at least 70%, 80%, 90%, 95%, or 100% sequence identity to SEQ ID NO. 2.

36. The composition of any one of paragraphs 33 to 35, wherein the arrestin polypeptide comprises at least a mutation selected from the group consisting of L377A, V378A, F379A, and a combination thereof.

37. The composition of any one of paragraphs 33 to 35, wherein the arrestin polypeptide comprises at least a mutation selected from the group consisting of L377A, V378A, F379A, K261Q, E350H, Q332K, or a combination thereof.

38. The composition of any one of paragraphs 1-37, wherein the arrestin polypeptide is an animal arrestin.

39. The composition of any one of paragraphs 1-37, wherein the opsin polypeptide is an animal arrestin.

40. The composition of any one of paragraphs 1-37, wherein the arrestin polypeptide is a human arrestin.

41. The composition of any one of paragraphs 1-37, wherein the opsin polypeptide is a human arrestin.

42. A composition comprising i) a first vector comprising a first nucleic acid encoding an opsin polypeptide and a second nucleic acid encoding an arrestin polypeptide; or ii) a first vector comprising a first nucleic acid encoding an opsin polypeptide, and a second vector comprising a second nucleic acid encoding an arrestin polypeptide.

43. The composition of paragraph 42, wherein the arrestin polypeptide is a mutant or a wild type.

44. The composition of paragraph 42, wherein the opsin polypeptide is a mutant or a wildtype.

45. The composition of paragraph 42, wherein the second nucleic acid encodes a mutant arrestin polypeptide.

46. A composition comprising a first vector having a nucleic acid encoding an opsin polypeptide, a second vector having a nucleic acid encoding a G-protein coupled receptor kinase GRK1 polypeptide, and a third vector having a nucleic acid encoding an arrestin.

47. A composition comprising a vector having a first nucleic acid encoding an opsin polypeptide, a second nucleic acid encoding a G-protein coupled receptor kinase GRK1 polypeptide, and a third nucleic acid encoding an arrestin.

48. The composition of paragraph 46 or 47, further comprising a nucleic acid encoding an arrestin polypeptide.

49. The composition of any one of paragraphs 1-48, wherein the opsin polypeptide comprises a mutation associated with a C-terminal phosphorylation site.

50. The composition of any one of paragraphs 1-49, wherein the mutant arrestin polypeptide comprises a mutation associated with affinity for unphosphorylated opsin.

51. The composition of any one of paragraphs 1-50, wherein the vector is a viral vector.

52. The composition of paragraph 51, wherein said viral vector is an adeno-associated viral (AAV) vector or a modified AAV thereof.

53. The composition of paragraph 51 or 52, wherein the vector is selected from the group consisting of AAV1, AAV2, AAV3, AAV4, AAV5, AAV6, AAV7, AAV8, AAV9, and AAV10.

54. The composition of any one of paragraphs 1-53, wherein the composition is suitable for ocular or subretinal administration.

55. A method of increasing deactivation of an opsin polypeptide, comprising administering an effective amount of a composition comprising an opsin polypeptide comprises at least one mutation increasing a rate of Schiff base hydrolysis or a rate of meta-II decay.

56. A method of increasing a temporal resolution of an opsin light response, comprising administering an effective amount of a composition comprising an opsin polypeptide comprises at least one mutation increasing a rate of Schiff base hydrolysis or a rate of meta-II decay.

57.. The method of paragraph 55 or 56, wherein the opsin polypeptide comprises at least a mutation selected from the group consisting of L59Q, Y74F, E122Q, A132L, A132S, Y136F, I189P, Y227F, Y306F, and a combination thereof.

58. The method of paragraph 55 or 56, wherein the opsin polypeptide comprises at least one mutation associated with one or more phosphorylation sites.

59. The method of paragraph 58, wherein the mutation is associated with a C-terminal phosphorylation site.

60. The method of paragraph 58, wherein the mutation results in amino acid substitution, deletion, or addition at the one or more phosphorylation sites of the opsin polypeptide.

61. The method of paragraph 58, wherein the opsin polypeptide comprises a sequence having at least 70%, 80%, 90%, 95%, or 100% sequence identity to SEQ ID NO. 4.

62. A method of increasing deactivation of an opsin polypeptide, comprising contacting the opsin polypeptide to an arrestin polypeptide having a mutation that increases binding between the arrestin polypeptide and unphosphorylated opsin.

63. A method of increasing a temporal resolution of an opsin light response, comprising administering an effective amount of a composition comprising an arrestin polypeptide having a mutation that increases binding between the arrestin polypeptide and unphosphorylated opsin.

64. The method of paragraph 62 or 63, wherein the arrestin polypeptide comprises a sequence having at least 70%, 80%, 90%, 95%, or 100% sequence identity to SEQ ID NO. 1.

65. The method of paragraph 62 or 63, wherein the arrestin polypeptide comprises a sequence having at least 70%, 80%, 90%, 95%, or 100% sequence identity to SEQ ID NO. 2.

66. The method of paragraph 62 or 63, wherein the arrestin polypeptide comprises at least a mutation selected from the group consisting of L377A, V378A, F379A, and a combination thereof.

67. The method of paragraph 62 or 63, wherein the arrestin polypeptide comprises at least a mutation selected from the group consisting of L377A, V378A, F379A, K261Q, E350H, Q332K, or a combination thereof.

68. A method of providing photoreceptor function to an inner retinal cell, comprising administering an effective amount of the composition of any one of paragraphs 1-54.

69. A method of increasing a temporal resolution of an opsin light response comprising administering a polynucleotide comprising a nucleic acid sequence encoding the composition of any one of paragraphs 1-54.

70. A method of treating a retinal degenerative condition in a subject in need thereof, comprising administering an effective amount of the composition of any one of paragraphs 1-54.

71. The method of paragraph 69, wherein the retinal degenerative condition is a retinal dystrophy, a rod dystrophy, a rod-cone dystrophy, a cone-rod dystrophy, a cone dystrophy and a macular dystrophy; other forms of retinal or macular degeneration, an ischaemic condition, uveitis or a condition resulting from loss of photoreceptor ability.

72. The method according to any one of paragraphs 55-71, wherein the composition is an injectable liquid.

73. The method according to any one of paragraphs 55-72, wherein the composition is administered by injection, preferably an intra-ocular injection, preferably a sub-retinal or intravitreal injection.

74. A method for generating photoactivatable cells comprising administering a polynucleotide comprising a nucleic acid sequence encoding the composition of any one of paragraphs 1-54.

75. The method of paragraph 74, wherein the polynucleotide further comprises a polynucleotide expression vector.

76. The method of paragraph 74 or 75, wherein the polynucleotide further comprises a nucleic acid sequence encoding a GRK1 G-protein coupled receptor kinase.

77. The method of any one of paragraphs 74 to 76, wherein the polynucleotide is incorporated into a genome of the cell.

78. The method of any one of paragraphs 74-1, wherein the polynucleotide is constitutively expressed.

79. The method of any one of paragraphs 74-1, wherein the polynucleotide is transiently expressed.

80. The method of any one of paragraphs 74-1, wherein the cell comprises a neuronal cell.

81. The method of any one of paragraphs 74-1, wherein the cell comprises a neuronal stem cell.

82. The method of any one of paragraphs 74-1, wherein the cell comprises an inner retinal cell.

83. The method of paragraph 82, wherein the inner retinal cell is an ON-bipolar cell, an OFF-bipolar cell, a horizontal cell, a ganglion cell and/or an amacrine cell.

84. The method of paragraph 83, wherein the retinal degenerative condition is a retinal dystrophy, a rod dystrophy, a rod-cone dystrophy, a cone-rod dystrophy, a cone dystrophy, a macular dystrophy, another form of retinal or macular degeneration, an ischaemic condition, an uveitis or a condition resulting from a loss of photoreceptor function.

85. The method according to any one of paragraphs 55-84, wherein the composition is administered by injection, preferably an intra-ocular injection, preferably a sub-retinal or intravitreal injection.

What is claimed is:

1. A composition comprising an opsin polypeptide and an arrestin polypeptide, wherein at least one of the opsin polypeptide and the arrestin polypeptide comprises a mutation that increases a temporal resolution of the opsin polypeptide’s response to light.

2. The composition of claim 1, wherein the arrestin is a mutant form.

3. The composition of claim 1, wherein the arrestin is a wild type.

4. The composition of any one of claims 1-3, wherein the opsin is a wild type.

5. The composition of any one of claims 1-3, wherein the opsin is a mutant form.

6. The composition of any one of claims 1 or 2, or 4 or 5 when dependent upon claims 1 or 2, wherein the arrestin polypeptide comprises a mutation that allows for phosphorylation independent binding between the opsin polypeptide and arrestin polypeptide.

7. The composition of any one of claims 1 to 6, wherein the opsin polypeptide is operably linked to the arrestin polypeptide through a linker.

8. The composition of claim 7, wherein the linker is a flexible linker, a rigid linker, a semi-flexible linkerER/K linker, or a combination thereof.

9. The composition of claim 7, wherein the linker is a flexible glycine-serine linker, a rigid alpha-helix forming linkers, a semi-flexible linker having a rigid linker with flexible ends, and ER/K linkers, or a combination thereof.

10. The composition of any one of claims 1 to 6, wherein the opsin polypeptide and the arrestin polypeptide are separate.

11. The composition of any one of claims 1 to 10, wherein the opsin is minimally phosphorylated by a G-protein coupled receptor kinase.

12. The composition of any one of claims 1 to 3, or 5 to 11 when dependent upon claims 1 or 2, wherein the opsin polypeptide comprises at least one mutation increasing a rate of Schiff base hydrolysis or a rate of meta-II decay.

13. The composition of claim 12, wherein the opsin polypeptide comprises at least one mutation selected from the group consisting of L59Q, Y74F, E122Q, A132L, A132S, Y136F, I189P, Y227F, Y306F, and a combination thereof.

14. The composition of any one of claims 1 or 2, or 4 to 11 when dependent upon claims 1 or 2 wherein the opsin polypeptide comprises a mutation associated with a C terminal phosphorylation site.

15. The composition of any one of claims 1 to 3 or 5, or claims 6 to 13 when dependent upon claims 1 to 3 or 5, wherein the opsin polypeptide comprises a mutation associated with a C-terminal phosphorylation site.

16. The composition of claim 14 wherein the opsin polypeptide comprises a substitution mutation at position S333A, T336A, T340A, T342A or S343A, or a combination thereof.

17. The composition of claim 14 or 15 wherein the opsin polypeptide comprises a substitution mutation at position S333A, T336A, T340A, T342A and S343A.

18. The composition of any one of claims 1 to 3 or 5, or claims 6 to 16 when dependent upon claims 1 to 3 or 5, wherein the opsin polypeptide comprises a sequence having at least 70%, 80%, 90%, 95%, or 100% sequence identity to SEQ ID NO. 4.

19. The composition of any one of claims 1, 2 or 4 to 18 when dependent upon claims 1 or 2, wherein the arrestin polypeptide comprises a sequence having at least 70%, 80%, 90%, 95%, or 100% sequence identity to SEQ ID NO. 1 or 2.

20. The composition of any one of claims 1, 2 or 4 to 19 when dependent upon claims 1 or 2, wherein the arrestin polypeptide comprises a mutation associated with increased affinity for unphosphorylated opsin.

21. The composition of any one of claims 1, 2 or 4 to 20 when dependent upon claims 1 or 2, wherein the arrestin polypeptide comprises at least a mutation selected from the group consisting of L377A, V378A, F379A, and a combination thereof.

22. The composition of claim 22 wherein the arrestin polypeptide comprises the mutations 377A, V378A and F379A (Arr3A).

23. The composition of any one of claims 1, 2 or 4 to 22 when dependent upon claims 1 or 2 wherein the arrestin polypeptide comprises at least a mutation selected from the group consisting of L377A, V378A, F379A, K261Q, E350H, Q332K, or a combination thereof.

24. The composition of claim 23 wherein the arrestin polypeptide comprises the mutations K261Q, E350H, and Q332K (ArrKEQ3A).

25. A composition comprising an opsin polypeptide operably linked to an arrestin polypeptide, preferably wherein the opsin polypeptide and the arrestin polypeptide are as defined in any one of claims 2 to 20.

26. The composition of claim 25, wherein the linker is a flexible linker, a rigid linker, a semi-flexible linker ER/K linker, or a combination thereof.

27. The composition of claim 25, wherein the linker is a flexible glycine-serine linker, a rigid alpha-helix forming linkers, a semi-flexible linker having a rigid linker with flexible ends, and ER/K linkers, or a combination thereof.

28. A composition comprising i) a first vector comprising a first nucleic acid encoding an opsin polypeptide and a second nucleic acid encoding an arrestin polypeptide; or ii) a first vector comprising a first nucleic acid encoding an opsin polypeptide, and a second vector comprising a second nucleic acid encoding an arrestin polypeptide.

29. A composition according to claim 28 wherein the opsin polypeptide and the arrestin polypeptide are as defined in any one of claims 2 to 20.

30. A composition according to claim 28 or 29, wherein a first vector of i) further comprises a nucleic acid encoding a G protein coupled receptor kinase GRK1 polypeptide; or a first or second vector of ii) further comprises a nucleic acid encoding a G protein coupled receptor kinase GRK1 polypeptide; or the composition comprises a further vector comprising a nucleic acid encoding a G protein coupled receptor kinase GRK1 polypeptide.

31. The composition of any one of claims 28 to 30, wherein a vector comprises a linker to operably link the opsin polypeptide and the arrestin polypeptide.

32. The composition of claim 31 wherein a vector of i) comprises a linker between the first and second nucleic acid sequences; or a nucleic acid sequence of a vector of ii) comprises a linker.

33. The composition of claim 32 wherein the linker is a flexible linker, a rigid linker, a semi-flexible linker ER/K linker, or a combination thereof.

34. The composition of claim 33, wherein the linker is a flexible glycine-serine linker, a rigid alpha-helix forming linker, a semi-flexible linker having a rigid linker with flexible ends, and an ER/K linker, or a combination thereof.

35. A composition according to any one of claims 28 to 34 wherein one or more of the first, second or further vectors is a viral vector.

36. A composition according to any one of claims 28 to 35 wherein the first, second and further vectors are viral vectors.

37. A composition according to any one of claims 28 to 36 wherein the viral vector is AAV, preferably AAV2.

38. A composition according to any one of the preceding claims wherein the arrestin is an animal or a human arrestin.

39. A composition according to any one of the preceding claims wherein the opsin is an animal or a human opsin.

40. A composition according to any one of the preceding claims, wherein the composition is suitable for ocular or subretinal administration.

41. An injectable liquid comprising a composition of any one of the preceding claims.

42. A method of providing photoreceptor function to an inner retinal cell, comprising administering to the cell an effective amount of the composition of any one of claims 1-41.

43. A method of increasing deactivation of an opsin polypeptide in a cell, comprising administering an effective amount of a composition according to claim 12 or 13.

44. A method of increasing a temporal resolution of an opsin light response in a cell comprising administering to the cell a polynucleotide comprising a nucleic acid sequence encoding the composition of claim 22.

45. A method of treating a retinal degenerative condition in a subject in need thereof, comprising administering an effective amount of the composition of any one of claims 1-41.

46. A method according to claim 44 wherein the retinal degenerative condition is a retinal dystrophy, a rod dystrophy, a rod-cone dystrophy, a cone-rod dystrophy, a cone dystrophy and a macular dystrophy; other forms of retinal or macular degeneration, an ischaemic condition, uveitis or a condition resulting from loss of photoreceptor ability.

47. A method according to claim 44 or 45 wherein the composition is administered by injection, preferably intra-ocular injection, preferably sub-retinal or intra-vitreal injection.

48. A method for generating photoactivatable cells comprising administering to the cell a composition according to any one claims 30 to 41.

49. A method according to claim 48 wherein a nucleic acid sequence is incorporated into a genome of the cell.

50. A method according to claim 48 or 49 wherein a nucleic acid sequence is constitutively expressed or transiently expressed.

51. The method of any one of claims 48 to 50, wherein the cell comprises a neuronal cell, a neuronal stem cell, or an inner retinal cell including an ON-bipolar cell, an OFF-bipolar cell, a horizontal cell, a ganglion cell and/or an amacrine cell.
